(19) United States
(12) Patent Application Publication
(10) Pub. No.: US 2003/0235915 A1
(43) Pub. Date: Dec. 25, 2003

(54) Human-nucleic acid sequences from breast tumor tissue

(75) Inventors: Thomas Specht, Berlin (DE); Bernd Hinzmann, Berlin (DE); Armin Schmitt, Berlin (DE); Christian Pilarsky, Schonfeld (DE); Edgar Dahl, Potsdam (DE); Andre Rosenthal, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Metagen Gesellschaft Fur Genomforschung MBH, Berlin (DE)

(21) Appl. No.: 10/131,410

(22) Filed: Apr. 25, 2002

Related U.S. Application Data
(62) Division of application No. 09/646,673, filed on Sep. 20, 2000.

(30) Foreign Application Priority Data
Mar. 20, 1998 (DE) 198 13 839.3

(51) Int. Cl. C12N 15/85; C07H 21/04; C12P 21/02; C12N 5/06

(52) U.S. Cl. 435/455; 435/69.1; 435/320.1; 435/325; 536/23.2

(57) ABSTRACT

Human nucleic acid sequences—mRNA, cDNA, genomic sequences—from breast tumor tissue, which code for gene products or parts thereof, and their use, are described. In addition, the polypeptides that can be obtained by way of the sequences and their use are described.

The invention relates to human nucleic acid sequences from breast tumor tissue, which code for gene products or parts thereof, their functional genes that code at least one bioactive polypeptide and their use.

In addition, the invention relates to the polypeptides that can be obtained via the sequences and their use.

One of the main causes of death in women is breast cancer, for control of which new therapies are necessary. Previously used therapies, such as, e.g., chemotherapy, hormone therapy or surgical removal of tumor tissue, frequently do not result in a complete cure.

The cancer phenomenon often goes along with overexpression or underexpression of certain genes in degenerated cells, it still being unclear whether these altered expression rates are the cause or the result of the malignant transformation. Identification of these genes would be an important step for development of new therapies against cancer. Spontaneous formation of cancer is often preceded by a host of mutations. They can have the most varied effects on the expression pattern in the affected tissue, such as, e.g., underexpression or overexpression, but also expression of shortened genes. Several such changes due to these mutation cascades can ultimately lead to malignant degeneration. The complexity of these relationships makes an experimental approach very difficult.

A database that consists of so-called ESTs is used to look for tumor-related candidate genes, i.e., genes that can be considered as the cause or the result of malignant degenerations in normal human tissue. ESTs (expressed sequence tags) are sequences of cDNAs, i.e., mRNAs transcribed in reverse, therefore molecules that reflect gene expression. The EST sequences are determined for normal and degenerated tissue. These databases are offered to some extent commercially by various companies. The ESTs of the LifeSeq database, which is used here, are generally between 150 and 350 nucleotides long. They represent a pattern that is unmistakable for a certain gene, although this gene is normally very much longer (>2000 nucleotides). By comparison of the expression patterns of normal and tumor tissue, ESTs can be identified which are important for tumor formation and proliferation. There is, however, the following problem: Since the EST sequences that are found can belong to different regions of an unknown gene due to different constructions of cDNA libraries, in this case a completely incorrect ratio of the occurrence of these ESTs in the respective tissue would arise. This would only be noticed when the complete gene is known and thus ESTs can be assigned to the same gene.

It has now been found that this error possibility can be reduced if all ESTs from the respective tissue type are assembled beforehand, before the expression patterns are compared to one another. Overlapping ESTs of the same gene were thus combined into longer sequences (see FIG. 1, FIG. 2a and FIG. 3). This lengthening and thus coverage of an essentially larger gene region in each of the respective bases are intended to largely avoid the above-described error. Since there were no existing software products for this purpose, programs for assembling genomic sections were employed, which were used modified and to which our own programs were added. A flow chart of the assembly procedure is shown in FIGS. 2b1-2b4.

Nucleic acid sequences Seq. ID No. 2-67, 149-161, and 201-202, which play a part as candidate genes in breast tumors, have now been found.

Nucleic acid sequences Seq. ID No. 3, 7, 9, 15, 17, 18, 21, 25, 27, 33, 35, 36, 38, 39, 40, 42, 43, 44, 46, 47, 48, 50, 51, 52, 53, 54, 55, 57, 58, 59, 61, 62, 63, 65, 66, 67, 150-153, 155-159, and 160-161 are of special interest.

The invention thus relates to nucleic acid sequences that code a gene product or a part thereof, comprising

a) a nucleic acid sequence selected from the group of nucleic acid sequences Seq. ID No. 3, 7, 9, 15, 17, 18, 21, 25, 27, 33, 35, 36, 38, 39, 40, 42, 43, 44, 46, 47, 48, 50, 51, 52, 53, 54, 55, 57, 58, 59, 61, 62, 63, 65, 66, 67, 150-153, 155-159, 160-161

b) an allelic variation of the nucleic acid sequences named under a) or

c) a nucleic acid sequence that is complementary to the nucleic acid sequences named under a) or b).

In addition, the invention relates to a nucleic acid sequence according to one of the sequences Seq. ID No. 3, 7, 9, 15, 17, 18, 21, 25, 27, 33, 35, 36, 38, 39, 40, 42, 43, 44, 46, 47, 48, 50, 51, 52, 53, 54, 55, 57, 58, 59, 61, 62, 63, 65, 66, 67, 150-153, 155-159, and 160-161 or a complementary or allelic variant thereof and the nucleic acid sequences thereof, which have 90% to 95% homology to a human nucleic acid sequence.

The invention also relates to nucleic acid sequences Seq. ID No. 2 to Seq. ID No. 67 as well as Seq. ID No. 149 to Seq. ID No. 201-202, which are expressed elevated in breast tumor tissue.

The invention further relates to nucleic acid sequences comprising a portion of the above-mentioned nucleic acid sequences in such a sufficient amount that they hybridize with sequences Seq. ID No. 3, 7, 9, 15, 17, 18, 21, 25, 27, 33, 35, 36, 38, 39, 40, 42, 43, 44, 46, 47, 48, 50, 51, 52, 53, 54, 55, 57, 58, 59, 61, 62, 63, 65, 66, 67, 150-153, 155-159 and 160-161.

The nucleic acid sequences according to the invention generally have a length of at least 50 to 4500 bp, preferably a length of at least 150 to 4000 bp, especially a length of 450 to 3500 bp.

With the partial sequences Seq. ID No. 3, 7, 9, 15, 17, 18, 21, 25, 27, 33, 35, 36, 38, 39, 40, 42, 43, 44, 46, 47, 48, 50, 51, 52, 53, 54, 55, 57, 58, 59, 61, 62, 63, 65, 66, 67, 150-153, 155-159, and 160-161 according to the invention, expression cassettes can also be built using current process practice, whereby on the cassette at least one of the nucleic acid sequences according to the invention is combined with at least one control or regulatory sequence generally known to one skilled in the art, such as, e.g., a suitable promoter. The sequences according to the invention can be inserted in a sense or antisense orientation.

A large number of expression cassettes or vectors and promoters which can be used are known in the literature.

Expression cassettes or vectors are defined as:

1. bacterial, such as, e.g., phagescript, pBs, φX174, pBluescript SK, pBs KS, pNH8a, pNH16a, pNH18a, pNH46a (Stratagene), pTrc99A, pKK223-3, pKK233-3, pDR540, pRIT5 (Pharmacia), 2. eukaryotic, such as, e.g., pWLneo, pSV2cat, pOG44, pXT1, pSG (Stratagene), pSVK3, pBPV, pMSG, pSVL (Pharmacia).

A control or regulatory sequence is defined as suitable promoters. Here, two preferred vectors are the pKK232-8 and the PCM7 vector. In particular, the following promoters are intended: lacI, lacZ, T3, T7, gpt, lambda P_{R}, trc, CMV, HSV thymidine-kinase, SV40, LTRs from retrovirus and mouse metallothionein-I.

The DNA sequences located on the expression cassette can code a fusion protein which comprises a known protein and a bioactive polypeptide fragment.

The expression cassettes are likewise the subject matter of this invention.

The nucleic acid fragments according to the invention can be used to produce full-length genes. The genes that can be obtained are likewise the subject matter of this invention.

The invention also relates to the use of the nucleic acid sequences according to the invention and the gene fragments that can be obtained from use.

The nucleic acid sequences according to the invention can be moved with suitable vectors into host cells, in which as the heterologous part, the genetic information which is contained on the nucleic acid fragments and which is expressed is located.

The host cells containing the nucleic acid fragments are likewise the subject matter of this invention.

Suitable host cells are, e.g., prokaryotic cell systems such as E. coli or eukaryotic cell systems such as animal or human cells or yeasts.

The nucleic acid sequences according to the invention can be used in the sense or antisense form.

Production of polypeptides or their fragments is done by cultivation of the host cells according to current cultivation methods and subsequent isolation and purification of the peptides or fragments, likewise using current methods. The invention further relates to nucleic acid sequences, which code at least a partial sequence of a bioactive polypeptide.

This invention further relates to polypeptide partial sequences, so-called ORF (open-reading-frame)-peptides according to the sequence protocols Seq. ID No. 71-79, 81-117, 119-121, 123, 126, 128-147, 162-168, 172, 174, 177-180, 183-185, 187, 190, and 192-198.

The invention further relates to the polypeptide sequences that have at least 80% homology, especially 90% homology to the polypeptide partial sequences of Seq. ID No. 71-179, 81-117, 119-121, 123, 126, 128-147, 162-168, 172, 174, 177-180, 183-185, 187, 190, and 192-198 according to the invention.

The invention also relates to antibodies that are directed against a polypeptide or a fragment thereof and that are coded by the nucleic acids of sequences Seq. ID No. 71-79, 81-117, 119-121, 123, 126, 128-147, 162-168, 172, 174, 177-180, 183-185, 187, 190, and 192-198 according to the invention.

Antibodies are defined especially as monoclonal antibodies.

The polypeptides of sequences Seq. ID No. 71-79, 81-117, 119-121, 123, 126, 128-147, 162-168, 172, 174, 177-180, 183-185, 187, 190, and 192-198 according to the invention can also be used as tools for finding active ingredients against breast cancer, which is likewise the subject matter of this invention.

Likewise the subject matter of this invention is the use of nucleic acid sequences according to sequences Seq. ID No. 3, 7, 9, 15, 17, 18, 21, 25, 27, 33, 35, 36, 38, 39, 40, 42, 43, 44, 46, 47, 48, 50, 51, 52, 53, 54, 55, 57, 58, 59, 61, 62, 63, 65, 66, 67, 150-153, 155-159, and 160-161 for expression of polypeptides, which can be used as tools for finding active ingredients against breast cancer.

The invention also relates to the use of the found polypeptide partial sequences Seq. ID No. 71-79, 81-117, 119-121, 123, 126, 128-147, 162-168, 172, 174, 177-180, 183-185, 187, 190, and 192-198 for the production of a pharmaceutical agent for treatment of breast cancer.

The invention also relates to pharmaceutical agents that contain at least one polypeptide partial sequence Seq. ID No. 71-79, 81-117, 119-121, 123, 126, 128-147, 162-168, 172, 174, 177-180, 183-185, 187, 190, and 192-198.

The nucleic acid sequences found according to the invention can also be genomic or mRNA sequences.

The invention also relates to genomic genes, their exon and intron structures and their splice versions that can be obtained from cDNAs of sequences Seq. ID No. 3, 7, 9, 15, 17, 18, 21, 25, 27, 33, 35, 36, 38, 39, 40, 42, 43, 44, 46, 47, 48, 50, 51, 52, 53, 54, 55, 57, 58, 59, 61, 62, 63, 65, 66, 67, 150-153, 155-159, and 160-161, and their use together with suitable regulatory elements, such as suitable promoters and/or enhancers.

With the nucleic acids according to the invention (cDNA sequences), genomic BAC, PAC and Cosmid libraries are screened and specifically human clones are isolated via complementary base pairing (hybridization). The BAC, PAC and Cosmid clones isolated in this way are hybridized using fluorescence-in-situ hybridization on metaphase chromosomes and the corresponding chromosome sections on which the corresponding genomic genes lie are identified. BAC, PAC and Cosmid clones are sequenced in order to clarify the corresponding genomic genes in their complete structure (promoters, enhancers, silencers, exons and introns). BAC, PAC and Cosmid clones can be used as independent molecules for gene transfer (see FIG. 5).

The invention also relates to BAC, PAC and Cosmid clones containing functional genes and their chromosomal localization according to sequences Seq. ID No. 3, 7, 9, 15, 17, 18, 21, 25, 27, 33, 35, 36, 38, 39, 40, 42, 43, 44, 46, 47, 48, 50, 51, 52, 53, 54, 55, 57, 58, 59, 61, 62, 63, 65, 66, 67, 150-153, 155-159, and 160-161 for use as vehicles for gene transfer.

Meanings of Technical Terms and Abbreviations

Nucleic acids=Nucleic acids in this invention are defined as: mRNA, partial cDNA, full-length cDNA and genomic genes (chromosomes).

ORF=Open Reading Frame, a defined sequence of amino acids which can be derived from the cDNA sequence.

Contig=A set of DNA sequences that can be combined as a result of very great similarities into one sequence (consensus).

Singleton=A contig that contains only one sequence.

Module=Domain of a protein with a defined sequence, which represents one structural unit and which occurs in various proteins.

N=selectively the nucleotide A, T, G or C.

X=selectively one of the 20 naturally occurring amino acids.

Explanation of the Alignment Parameters

minimal initial match=minimal initial identity area

maximum pads per read=maximum number of insertions

maximum percent mismatch=maximum deviation in %

### EXPLANATION OF FIGURES

FIG. 1 shows the systematic gene search in the Incyte LifeSeq database.

FIG. 2a shows the principle of EST assembling

FIGS. 2b1-2b4 show the entire principle of EST assembling

FIG. 3 shows the in-silico subtraction of gene expression in various tissues

FIG. 4a shows the determination of tissue-specific expression via electronic Northern

FIG. 4b shows the electronic Northern

FIG. 5 shows the isolation of genomic BAC and PAC clones.

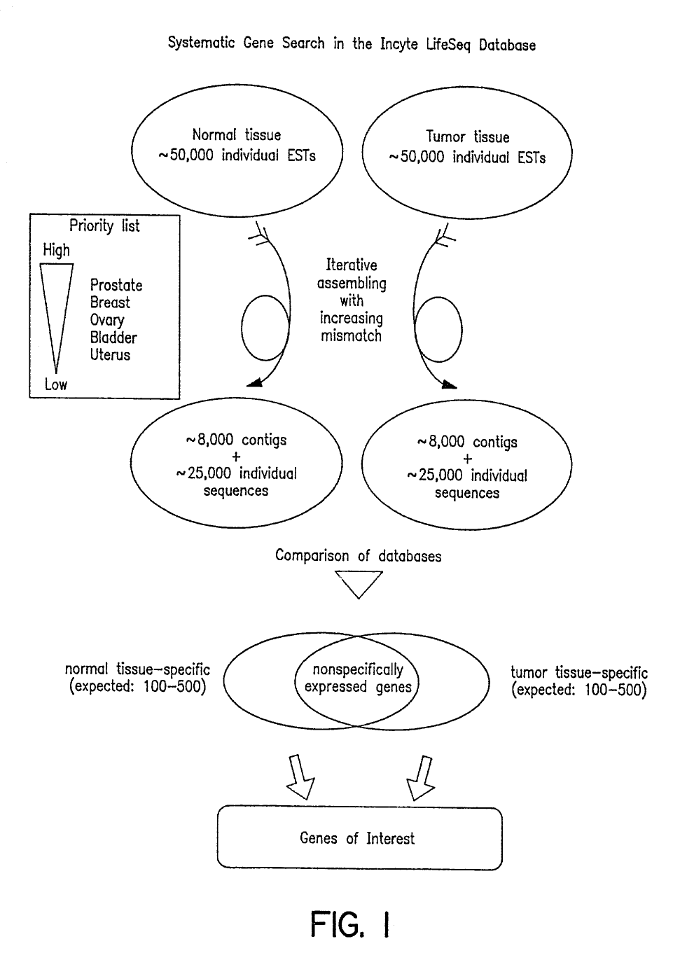

The following examples explain the production of the nucleic acid sequences according to the invention without limiting the invention to these examples and nucleic acid sequences.

### EXAMPLE 1

Search for Tumor-Related Candidate Genes

First, all ESTs of the corresponding tissue from the LifeSeq database (from October 1997) were extracted. They were then assembled by means of the GAP4 program of the Staden package with the parameters 0% mismatch, 8 pads per read and a minimal match of 20. The sequences (fails) not recorded in the GAP4 database were assembled first at 1% mismatch and then again at 2% mismatch with the database. Consensus sequences were computed from the contigs of the database that consisted of more than one sequence.

The singletons of the database, which consisted of only one sequence, were re-assembled at 2% mismatch with the sequences not recorded in the GAP4 database. In turn, the consensus sequences were determined for the contigs. All other ESTs were re-assembled at 4% mismatch. The consensus sequences were extracted once again and finally assembled with the previous consensus sequences and the singletons and the sequences not recorded in the database at 4% mismatch. The consensus sequences were formed and used with the singletons and fails as the initial basis for tissue comparisons. This procedure ensured that among the parameters used, all sequences represented gene regions independent of one another.

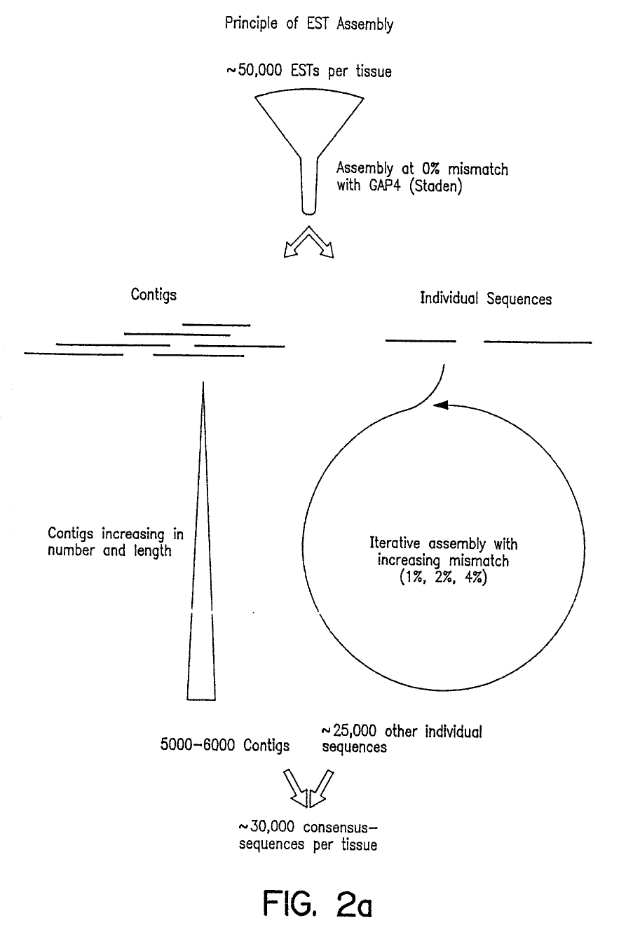

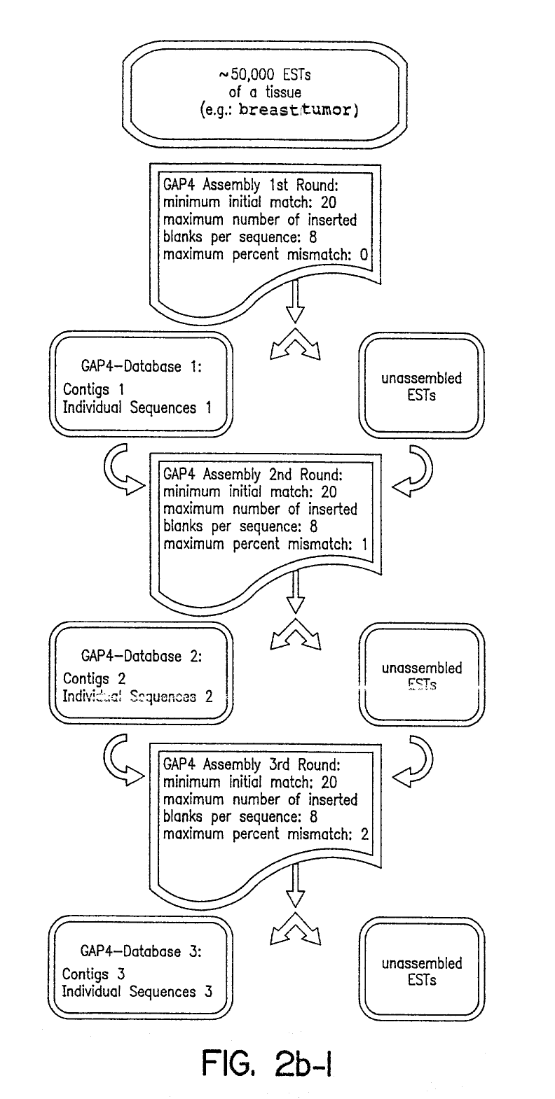

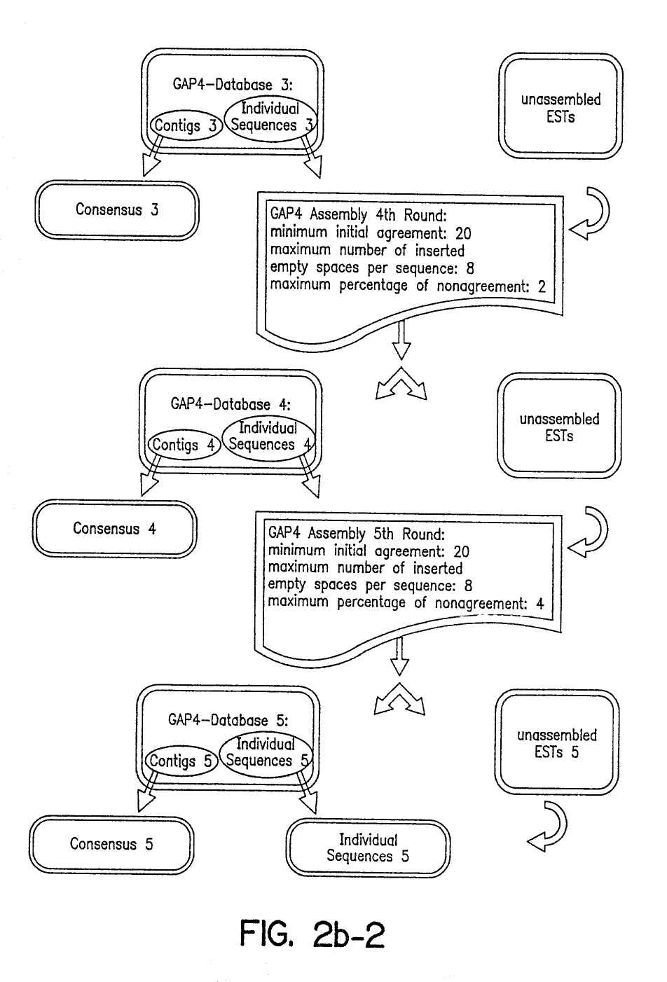

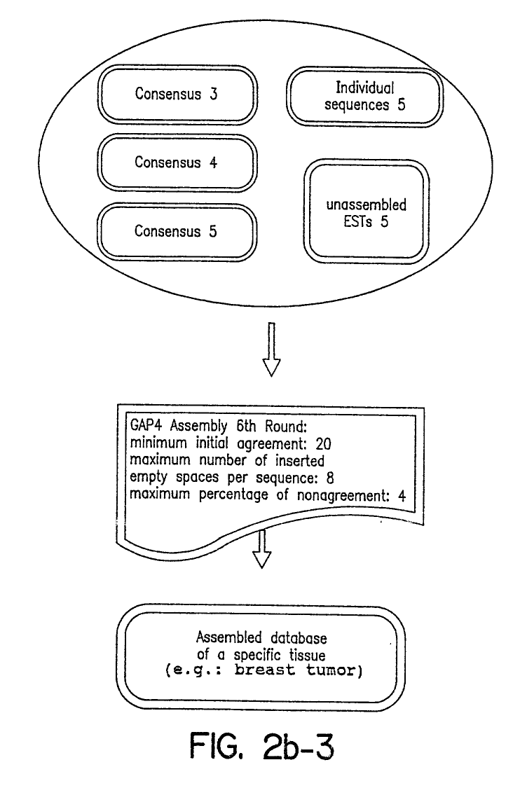

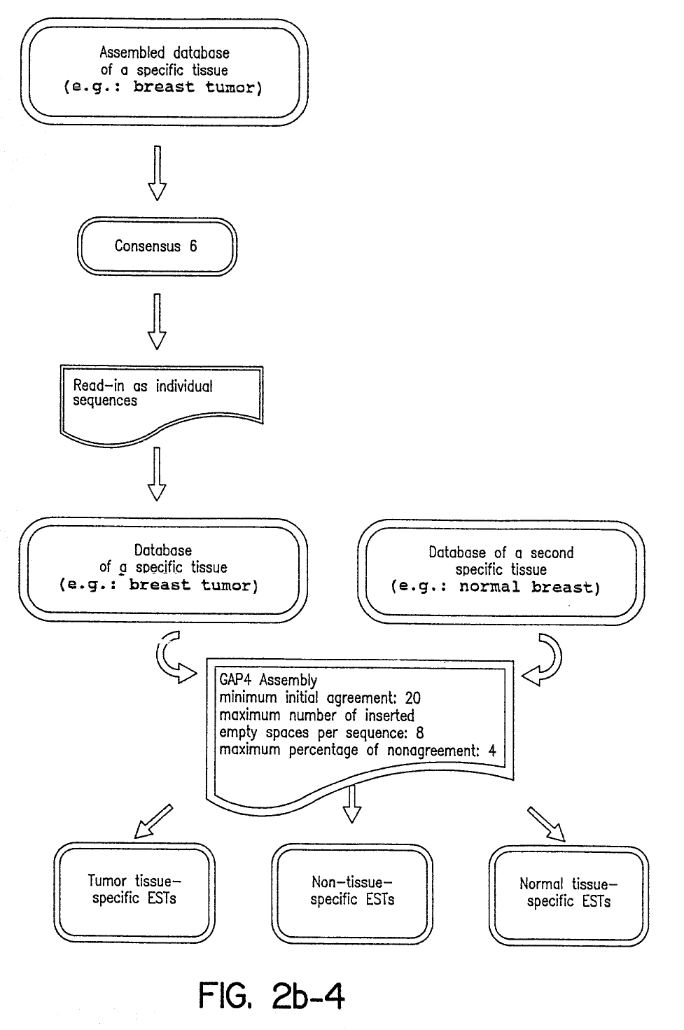

FIGS. 2b1-2b4 illustrate the lengthening of the breast tumor tissue ESTs.

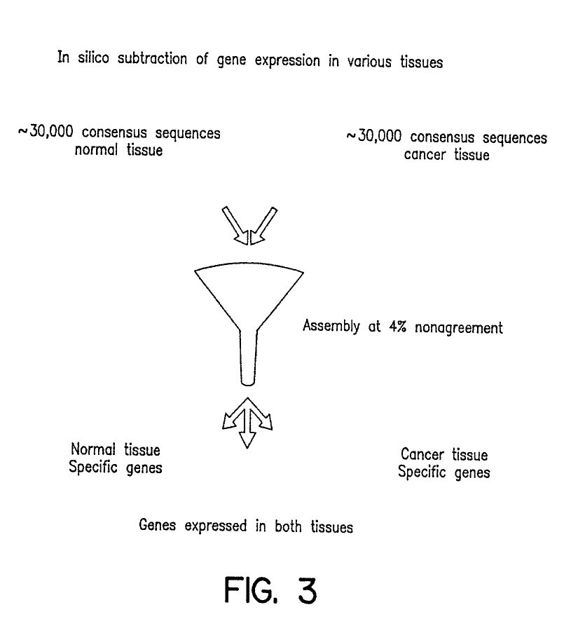

The sequences of the respective tissue assembled in this way were then compared to one another by means of the same program (FIG. 3). To do this, first all sequences of the first tissue were input into the database. (It was therefore important that they were independent of one another).

Then, all sequences of the second tissue were compared to all those of the first. The result was sequences that were specific to the first or the second tissue as well as those which occurred in both. In the latter, the ratio of the frequency of occurrence in the respective tissue was evaluated. All programs pertaining to the evaluation of the assembled sequences were themselves developed.

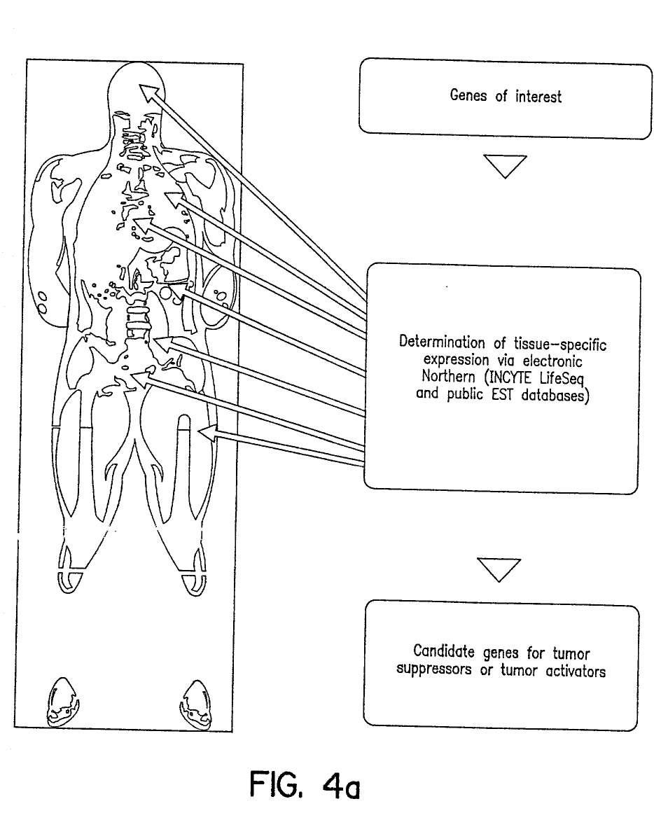

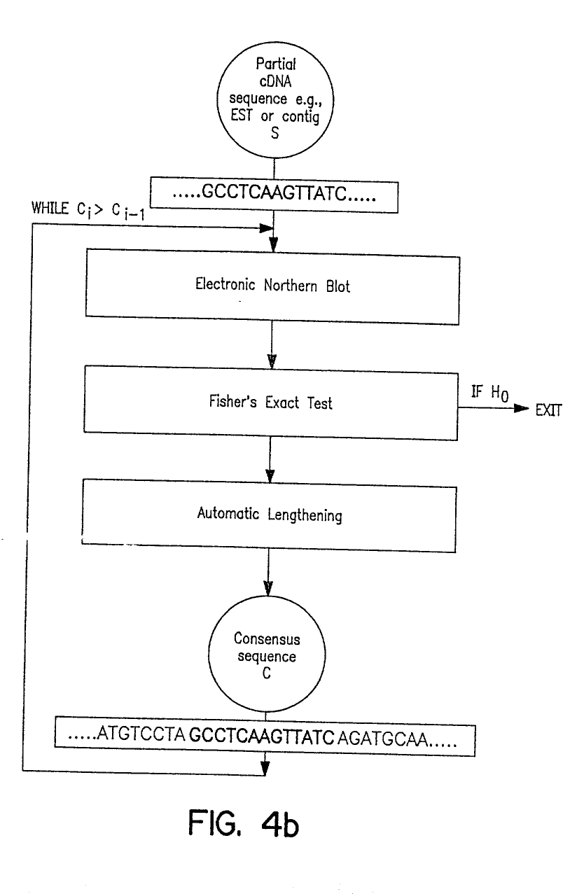

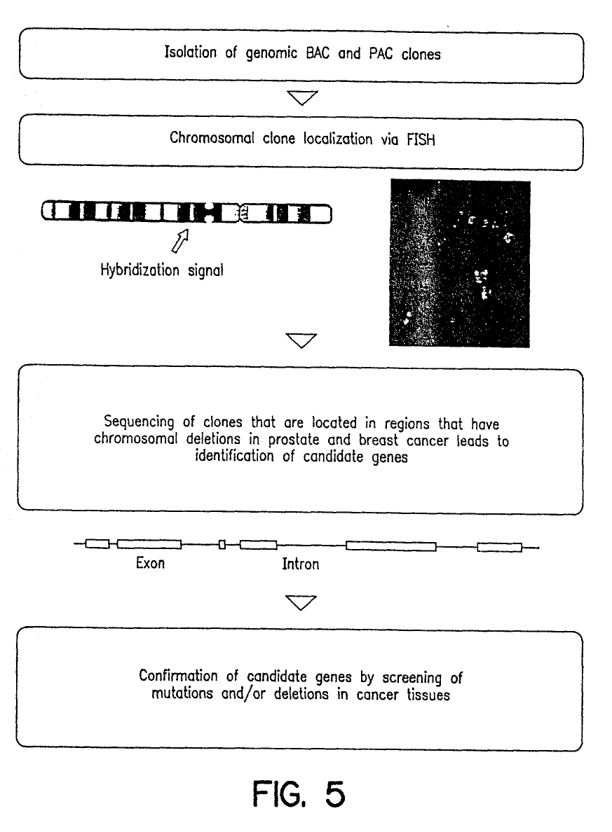

All sequences that occurred more than four times in respectively one of the compared tissues and all that occurred at least five times as often in one of the two tissues were further studied. These sequences were subjected to an electronic Northern (see Example 2.1), by which the distribution in all tumor and normal tissues was studied (see FIG. 4a and FIG. 4b). The relevant candidates were then lengthened using all Incyte ESTs and all ESTs of public databases (see Example 3). Then, the sequences and their translation into possible proteins were compared to all nucleotide and protein databases and were studied for possible regions that code for proteins.

### EXAMPLE 2

Algorithm for Identification and Lengthening of Partial cDNA Sequences with Altered Expression Pattern

An algorithm for finding overexpressed or underexpressed genes will be explained below. The individual steps are also summarized in a flow chart for the sake of clarity (see FIG. 4b).

2.1. Electronic Northern Blot

By means of a standard program for homology search, e.g., BLAST (Altschul, S. F.; Gish, W.; Miller, W.; Myers, E. W. and Lipman, D. J. (1990) J. Mol. Biol. 215, 403-410), BLAST2 (Altschul, S. F.; Madden, T. L.; Schäffer, A. A.; Zhang, J.; Zhang, Z.; Miller, W., and Lipman, D. J. (1997) Nucleic Acids Research 25 3389-3402) or FASTA (Pearson, W. R. and Lipman, D. J. (1988) Proc. Natl. Acad. Sci. USA 85 2444-2448), the homologous sequences in various EST libraries (private or public) arranged by tissues are determined for a partial DNA sequence S, e.g., an individual EST or a contig of ESTs. The (relative or absolute) tissue-specific occurrence frequencies of this partial sequence S which were determined in this way are called electronic Northern Blots.

2.1.1

Analogously to the procedure described under 2.1, the sequence Seq. ID No. 2 was found which occurs 9× more heavily in breast tumor tissue than in normal tissue.

The possible function of this gene region relates to a 17-kDa-interferon-inducible gene.

The result is as follows:

Electronic Northern for Seq. ID NO.: 2

| | | TU- | | |
|---|---|---|---|---|
| | | MOR | RATIOS | |
| | % freq. | % freq. | N/T | T/N |
| | NORMAL | | | |
| Bladder | 0.0000 | 0.0102 | 0.0000 | undef |
| Breast | 0.0053 | 0.0458 | 0.1165 | 8.5843 |
| Ovary | 0.0030 | 0.0338 | 0.0899 | 11.1243 |
| Endocrine tissue | 0.0000 | 0.0000 | undef | undef |
| Gastrointestinal | 0.0039 | 0.0143 | 0.2714 | 3.6843 |
| Brain | 0.0085 | 0.0099 | 0.8601 | 1.1626 |
| Hematopoietic | 0.0028 | 0.0000 | undef | 0.0000 |
| Skin | 0.0000 | 0.0000 | undef | undef |
| Hepatic | 0.0000 | 0.0000 | undef | undef |
| Heart | 0.0074 | 0.0000 | undef | 0.0000 |
| Testicles | 0.0000 | 0.0117 | 0.0000 | undef |
| Lungs | 0.0037 | 0.0024 | 1.5801 | 0.6329 |
| Stomach-esophagus | 0.0097 | 0.0077 | 1.2599 | 0.7937 |
| Muscle-skeleton | 0.0034 | 0.0120 | 0.2855 | 3.5025 |
| Kidneys | 0.0030 | 0.0000 | undef | 0.0000 |
| Pancreas | 0.0038 | 0.0000 | undef | 0.0000 |
| Penis | 0.0030 | 0.1066 | 0.0281 | 35.6161 |
| Prostate | 0.0048 | 0.0043 | 1.1186 | 0.8939 |
| Uterus | 0.0083 | 0.0000 | undef | 0.0000 |
| Breast hyperplasia | 0.0000 | | | |
| Small intestine | 0.0093 | | | |
| Prostatic hyperplasia | 0.0030 | | | |
| Seminal vesicles | 0.0089 | | | |
| Sensory organs | 0.0000 | | | |
| White blood cells | 0.0035 | | | |
| | FETUS | | | |
| Development | 0.0000 | | | |
| Gastrointestinal | 0.0031 | | | |
| Brain | 0.0000 | | | |
| Hematopoietic | 0.0039 | | | |
| Heart-blood vessels | 0.0000 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0228 | | | |
| Endocrine tissue | 0.0000 | | | |
| Fetal | 0.0082 | | | |
| Gastrointestinal | 0.0244 | | | |
| Hematopoietic | 0.0057 | | | |
| Skin-muscle | 0.0032 | | | |
| Testicles | 0.0000 | | | |
| Lungs | 0.0082 | | | |
| Nerves | 0.0010 | | | |
| Prostate | 0.0128 | | | |
| Sensory organs | 0.0000 | | | |

2.1.2

Analogously to the procedure described under 2.1, the sequence Seq. ID No. 5 was found which occurs 30× more heavily in breast tumor tissue than in normal tissue.

The possible function of this gene region relates to “macrophage migration inhibition factor related protein 14 (MRP-14).”

The result is as follows:

Electronic Northern for Seq. ID NO.: 5

| | | TU- | | |
|---|---|---|---|---|
| | | MOR | RATIOS | |
| | % freq. | % freq. | N/T | T/N |
| | NORMAL | | | |
| Bladder | 0.0093 | 0.1508 | 0.0616 | 16.2223 |
| Breast | 0.0013 | 0.0392 | 0.0340 | 29.4320 |
| Ovary | 0.0152 | 0.0000 | undef | 0.0000 |
| Endocrine tissue | 0.0018 | 0.0027 | 0.6698 | 1.4930 |
| Gastrointestinal | 0.0174 | 0.0048 | 3.6642 | 0.2729 |
| Brain | 0.0051 | 0.0055 | 0.9289 | 1.0765 |
| Hematopoietic | 0.0993 | 0.0000 | undef | 0.0000 |
| Skin | 0.0249 | 0.0000 | undef | 0.0000 |
| Hepatic | 0.0000 | 0.0129 | 0.0000 | undef |
| Heart | 0.0053 | 0.0000 | undef | 0.0000 |
| Testicles | 0.0061 | 0.0000 | undef | 0.0000 |
| Lungs | 0.0261 | 0.0307 | 0.8508 | 1.1753 |
| Stomach-esophagus | 0.1062 | 0.0230 | 4.6197 | 0.2165 |
| Muscle-skeleton | 0.0171 | 0.0180 | 0.9517 | 1.0508 |
| Kidneys | 0.0000 | 0.0068 | 0.0000 | undef |
| Pancreas | 0.0114 | 0.0000 | undef | 0.0000 |
| Penis | 0.1258 | 0.1600 | 0.7862 | 1.2720 |
| Prostate | 0.0000 | 0.0000 | undef | undef |
| Uterus | 0.0826 | 0.0000 | undef | 0.0000 |
| Breast hyperplasia | 0.0036 | | | |
| Small intestine | 0.0000 | | | |
| Prostatic hyperplasia | 0.0000 | | | |
| Seminal vesicles | 0.0178 | | | |
| Sensory organs | 0.0000 | | | |
| White blood cells | 0.1018 | | | |
| | FETUS | | | |
| Development | 0.0000 | | | |
| Gastrointestinal | 0.0062 | | | |
| Brain | 0.0000 | | | |
| Hematopoietic | 0.0000 | | | |
| Heart-blood vessels | 0.0000 | | | |
| Lungs | 0.0037 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0000 | | | |
| Endocrine tissue | 0.0000 | | | |
| Fetal | 0.0052 | | | |
| Gastrointestinal | 0.0122 | | | |
| Hematopoietic | 0.0057 | | | |
| Skin-muscle | 0.0000 | | | |
| Testicles | 0.0000 | | | |
| Lungs | 0.0410 | | | |
| Nerves | 0.0000 | | | |
| Prostate | 0.0449 | | | |
| Sensory organs | 0.0000 | | | |

2.1.3

Analogously to the procedure described under 2.1, the sequence Seq. ID No. 16 was found which occurs 30× more heavily in breast tumor tissue than in normal tissue.

The possible function of this gene region relates to human Tim23, which is located in the protein translocase complex of the inside mitochondrial membrane.

The result is as follows:

Electronic Northern for Seq. ID NO.: 16

| | | TU- | | |
|---|---|---|---|---|
| | | MOR | RATIOS | |
| | % freq. | % freq. | N/T | T/N |
| | NORMAL | | | |
| Bladder | 0.0046 | 0.0051 | 0.9092 | 1.0998 |
| Breast | 0.0013 | 0.0153 | 0.0874 | 11.4458 |
| Ovary | 0.0000 | 0.0026 | 0.0000 | undef |
| Endocrine tissue | 0.0091 | 0.0082 | 1.1163 | 0.8958 |
| Gastrointestinal | 0.0116 | 0.0095 | 1.2214 | 0.8187 |
| Brain | 0.0076 | 0.0055 | 1.3934 | 0.7177 |
| Hematopoietic | 0.0084 | 0.0000 | undef | 0.0000 |
| Skin | 0.0199 | 0.0000 | undef | 0.0000 |
| Hepatic | 0.0198 | 0.0065 | 3.0606 | 0.3267 |
| Heart | 0.0106 | 0.0000 | undef | 0.0000 |
| Testicles | 0.0183 | 0.0000 | undef | 0.0000 |
| Lungs | 0.0100 | 0.0024 | 4.2137 | 0.2373 |
| Stomach-esophagus | 0.0193 | 0.0000 | undef | 0.0000 |
| Muscle-skeleton | 0.0034 | 0.0060 | 0.5710 | 1.7513 |
| Kidneys | 0.0030 | 0.0000 | undef | 0.0000 |
| Pancreas | 0.0000 | 0.0000 | undef | undef |
| Penis | 0.0000 | 0.0000 | undef | undef |
| Prostate | 0.0048 | 0.0085 | 0.5593 | 1.7879 |
| Uterus | 0.0017 | 0.0071 | 0.2321 | 4.3088 |
| Breast hyperplasia | 0.0073 | | | |
| Small intestine | 0.0031 | | | |
| Prostatic hyperplasia | 0.0030 | | | |
| Seminal vesicles | 0.0089 | | | |
| Sensory organs | 0.0118 | | | |
| White blood cells | 0.0009 | | | |
| | FETUS | | | |
| Development | 0.0154 | | | |
| Gastrointestinal | 0.0000 | | | |
| Brain | 0.0125 | | | |
| Hematopoietic | 0.0000 | | | |
| Heart-blood vessels | 0.0041 | | | |
| Lungs | 0.0111 | | | |
| Kidneys | 0.0000 | | | |
| Prostate | 0.0499 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0068 | | | |
| Endocrine tissue | 0.0000 | | | |
| Fetal | 0.0064 | | | |
| Gastrointestinal | 0.0244 | | | |
| Hematopoietic | 0.0057 | | | |
| Skin-muscle | 0.0065 | | | |
| Testicles | 0.0156 | | | |
| Lungs | 0.0164 | | | |
| Nerves | 0.0030 | | | |
| Prostate | 0.0000 | | | |
| Sensory organs | 0.0000 | | | |

In an analogous procedure, the following Northerns were also found:

| | TU- | | |
|---|---|---|---|
| | MOR | RATIOS | |
| % freq. | % freq. | N/T | T/N |

Electronic Northern for SEQ. ID NO.: 3

| | | | | |
|---|---|---|---|---|
| | NORMAL | | | |
| Bladder | 0.0046 | 0.0128 | 0.3637 | 2.7495 |
| Breast | 0.0067 | 0.0174 | 0.3822 | 2.6162 |
| Ovary | 0.0061 | 0.0000 | undef | 0.0000 |
| Endocrine tissue | 0.0018 | 0.0082 | 0.2233 | 4.4791 |
| Gastrointestinal | 0.0039 | 0.0048 | 0.8143 | 1.2281 |
| Brain | 0.0339 | 0.0110 | 3.0964 | 0.3230 |
| Hematopoietic | 0.0112 | 0.0000 | undef | 0.0000 |
| Skin | 0.0149 | 0.0000 | undef | 0.0000 |
| Hepatic | 0.0050 | 0.0000 | undef | 0.0000 |
| Heart | 0.0064 | 0.0000 | undef | 0.0000 |
| Testicles | 0.0183 | 0.0000 | undef | 0.0000 |
| Lungs | 0.0137 | 0.0000 | undef | 0.0000 |
| Stomach-esophagus | 0.0097 | 0.0153 | 0.6300 | 1.5874 |
| Muscle-skeleton | 0.0086 | 0.0120 | 0.7138 | 1.4010 |
| Kidneys | 0.0030 | 0.0342 | 0.0868 | 11.5165 |
| Pancreas | 0.0057 | 0.0110 | 0.5143 | 1.9446 |
| Penis | 0.0030 | 0.0000 | undef | 0.0000 |
| Prostate | 0.0024 | 0.0085 | 0.2797 | 3.5758 |
| Uterus | 0.0116 | 0.0071 | 1.6246 | 0.6155 |
| Breast hyperplasia | 0.0036 | | | |
| Small intestine | 0.0093 | | | |
| Prostatic hyperplasia | 0.0030 | | | |
| Seminal vesicles | 0.0000 | | | |
| Sensory organs | 0.0118 | | | |
| White blood cells | 0.0061 | | | |
| | FETUS | | | |
| Development | 0.0000 | | | |
| Gastrointestinal | 0.0092 | | | |
| Brain | 0.0188 | | | |
| Hematopoietic | 0.0079 | | | |
| Heart-blood vessels | 0.0000 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0204 | | | |
| Ovary-uterus | 0.0091 | | | |
| Endocrine tissue | 0.0000 | | | |
| Fetal | 0.0093 | | | |
| Gastrointestinal | 0.0000 | | | |
| Hematopoietic | 0.0114 | | | |
| Skin-muscle | 0.0227 | | | |
| Testicles | 0.0000 | | | |
| Lungs | 0.0082 | | | |
| Nerves | 0.0291 | | | |
| Prostate | 0.0000 | | | |
| Sensory organs | 0.0000 | | | |

Electronic Northern for SEQ. ID NO.: 4

| | | | | |
|---|---|---|---|---|
| | NORMAL | | | |
| Bladder | 0.0093 | 0.0256 | 0.3637 | 2.7495 |
| Breast | 0.0133 | 0.0283 | 0.4704 | 2.1256 |
| Ovary | 0.0091 | 0.0104 | 0.8765 | 1.1409 |
| Endocrine tissue | 0.0274 | 0.0490 | 0.5582 | 1.7916 |
| Gastrointestinal | 0.0116 | 0.0095 | 1.2214 | 0.8187 |
| Brain | 0.0212 | 0.0164 | 1.2902 | 0.7751 |
| Hematopoietic | 0.0084 | 0.0000 | undef | 0.0000 |
| Skin | 0.0348 | 0.0000 | undef | 0.0000 |
| Hepatic | 0.0149 | 0.0259 | 0.5739 | 1.7426 |
| Heart | 0.0191 | 0.0137 | 1.3873 | 0.7208 |
| Testicles | 0.0122 | 0.0000 | undef | 0.0000 |
| Lungs | 0.0162 | 0.0165 | 0.9782 | 1.0223 |
| Stomach-esophagus | 0.0290 | 0.0153 | 1.8899 | 0.5291 |
| Muscle-skeleton | 0.0069 | 0.0120 | 0.5710 | 1.7513 |
| Kidneys | 0.0178 | 0.0068 | 2.6050 | 0.3839 |
| Pancreas | 0.0038 | 0.0221 | 0.1714 | 5.8337 |
| Penis | 0.0180 | 0.0000 | undef | 0.0000 |
| Prostate | 0.0214 | 0.0234 | 0.9152 | 1.0926 |
| Uterus | 0.0182 | 0.0214 | 0.8510 | 1.1751 |
| Breast hyperplasia | 0.0073 | | | |
| Small intestine | 0.0218 | | | |
| Prostatic hyperplasia | 0.0357 | | | |
| Seminal vesicles | 0.0178 | | | |
| Sensory organs | 0.0118 | | | |
| White blood cells | 0.0218 | | | |
| | FETUS | | | |
| Development | 0.0000 | | | |
| Gastrointestinal | 0.0154 | | | |
| Brain | 0.0125 | | | |
| Hematopoietic | 0.0000 | | | |
| Heart-blood vessels | 0.0123 | | | |
| Lungs | 0.0111 | | | |
| Kidneys | 0.0124 | | | |
| Prostate | 0.0499 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0183 | | | |
| Endocrine tissue | 0.0000 | | | |
| Fetal | 0.0076 | | | |
| Gastrointestinal | 0.0488 | | | |
| Hematopoietic | 0.0000 | | | |
| Skin-muscle | 0.0259 | | | |
| Testicles | 0.0000 | | | |
| Lungs | 0.0000 | | | |
| Nerves | 0.0090 | | | |
| Prostate | 0.0128 | | | |
| Sensory organs | 0.0000 | | | |

Electronic Northern for SEQ. ID NO.: 6

| | | | | |
|---|---|---|---|---|
| | NORMAL | | | |
| Bladder | 0.0000 | 0.0102 | 0.0000 | undef |
| Breast | 0.0053 | 0.0196 | 0.2718 | 3.6790 |
| Ovary | 0.0061 | 0.0052 | 1.1686 | 0.8557 |
| Endocrine tissue | 0.0018 | 0.0027 | 0.6698 | 1.4930 |
| Gastrointestinal | 0.0039 | 0.0095 | 0.4071 | 2.4562 |
| Brain | 0.0076 | 0.0033 | 2.3223 | 0.4306 |
| Hematopoietic | 0.0042 | 0.0000 | undef | 0.0000 |
| Skin | 0.0050 | 0.0000 | undef | 0.0000 |
| Hepatic | 0.0099 | 0.0129 | 0.7651 | 1.3069 |
| Heart | 0.0064 | 0.0000 | undef | 0.0000 |
| Testicles | 0.0000 | 0.0117 | 0.0000 | undef |
| Lungs | 0.0050 | 0.0142 | 0.3511 | 2.8478 |
| Stomach-esophagus | 0.0000 | 0.0230 | 0.0000 | undef |
| Muscle-skeleton | 0.0034 | 0.0000 | undef | 0.0000 |
| Kidneys | 0.0119 | 0.0137 | 0.8683 | 1.1517 |
| Pancreas | 0.0038 | 0.0055 | 0.6857 | 1.4584 |
| Penis | 0.0030 | 0.0000 | undef | 0.0000 |
| Prostate | 0.0167 | 0.0128 | 1.3051 | 0.7662 |
| Uterus | 0.0033 | 0.0142 | 0.2321 | 4.3088 |
| Breast hyperplasia | 0.0036 | | | |
| Small intestine | 0.0062 | | | |
| Prostatic hyperplasia | 0.0119 | | | |
| Seminal vesicles | 0.0089 | | | |
| Sensory organs | 0.0000 | | | |
| White blood cells | 0.0009 | | | |
| | FETUS | | | |
| Development | 0.0154 | | | |
| Gastrointestinal | 0.0062 | | | |
| Brain | 0.0000 | | | |
| Hematopoietic | 0.0000 | | | |
| Heart-blood vessels | 0.0041 | | | |
| Lungs | 0.0037 | | | |
| Kidneys | 0.0062 | | | |
| Prostate | 0.0249 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0023 | | | |
| Endocrine tissue | 0.0000 | | | |
| Fetal | 0.0047 | | | |
| Gastrointestinal | 0.0122 | | | |
| Hematopoietic | 0.0057 | | | |
| Skin-muscle | 0.0000 | | | |
| Testicles | 0.0000 | | | |
| Lungs | 0.0000 | | | |
| Nerves | 0.0000 | | | |
| Prostate | 0.0256 | | | |
| Sensory organs | 0.0000 | | | |

Electronic Northern for SEQ. ID NO.: 7

| | | | | |
|---|---|---|---|---|
| | NORMAL | | | |
| Bladder | 0.0186 | 0.0051 | 3.6370 | 0.2750 |
| Breast | 0.0147 | 0.0261 | 0.5606 | 1.7838 |
| Ovary | 0.0182 | 0.0234 | 0.7791 | 1.2836 |
| Endocrine tissue | 0.0456 | 0.0245 | 1.8605 | 0.5375 |
| Gastrointestinal | 0.0233 | 0.0190 | 1.2214 | 0.8187 |
| Brain | 0.0314 | 0.0230 | 1.3639 | 0.7332 |
| Hematopoietic | 0.0196 | 0.0378 | 0.5175 | 1.9325 |
| Skin | 0.0199 | 0.0000 | undef | 0.0000 |
| Hepatic | 0.0050 | 0.0388 | 0.1275 | 7.8416 |
| Heart | 0.0328 | 0.0275 | 1.1947 | 0.8371 |
| Testicles | 0.0183 | 0.0351 | 0.5224 | 1.9144 |
| Lungs | 0.0149 | 0.0284 | 0.5267 | 1.8986 |
| Stomach-esophagus | 0.0193 | 0.0460 | 0.4200 | 2.3811 |
| Muscle-skeleton | 0.0223 | 0.0480 | 0.4639 | 2.1554 |
| Kidneys | 0.0208 | 0.0205 | 1.0130 | 0.9871 |
| Pancreas | 0.0246 | 0.0055 | 4.4569 | 0.2244 |
| Penis | 0.0449 | 0.0267 | 1.6846 | 0.5936 |
| Prostate | 0.0167 | 0.0383 | 0.4350 | 2.2987 |
| Uterus | 0.0231 | 0.0214 | 1.0831 | 0.9233 |
| Breast hyperplasia | 0.0109 | | | |
| Small intestine | 0.0125 | | | |
| Prostatic hyperplasia | 0.0357 | | | |
| Seminal vesicles | 0.0356 | | | |
| Sensory organs | 0.0000 | | | |
| White blood cells | 0.0165 | | | |
| | FETUS | | | |
| Development | 0.0307 | | | |
| Gastrointestinal | 0.0247 | | | |
| Brain | 0.0375 | | | |
| Hematopoietic | 0.0118 | | | |
| Heart-blood vessels | 0.0204 | | | |
| Lungs | 0.0296 | | | |
| Kidneys | 0.0185 | | | |
| Prostate | 0.0249 | | | |
| Sensory organs | 0.0279 | | | |
| | STANDARDIZED/ | | | |
| | SUBTRACTED | | | |
| | LIBRARIES | | | |
| Breast | 0.0068 | | | |
| Ovary-uterus | 0.0068 | | | |
| Endocrine tissue | 0.0000 | | | |
| Fetal | 0.0093 | | | |
| Gastrointestinal | 0.0000 | | | |
| Hematopoietic | 0.0114 | | | |
| Skin-muscle | 0.0097 | | | |
| Testicles | 0.0078 | | | |
| Lungs | 0.0082 | | | |
| Nerves | 0.0100 | | | |
| Prostate | 0.0000 | | | |
| Sensory organs | 0.0155 | | | |

Electronic Northern for SEQ. ID NO.: 8

| | | | | |
|---|---|---|---|---|
| | NORMAL | | | |
| Bladder | 0.0139 | 0.0281 | 0.4959 | 2.0163 |
| Breast | 0.0080 | 0.0414 | 0.1931 | 5.1778 |
| Ovary | 0.0122 | 0.0260 | 0.4674 | 2.1393 |
| Endocrine tissue | 0.0274 | 0.0354 | 0.7728 | 1.2940 |
| Gastrointestinal | 0.0252 | 0.0333 | 0.7561 | 1.3226 |
| Brain | 0.0237 | 0.0175 | 1.3547 | 0.7382 |
| Hematopoietic | 0.0098 | 0.0000 | undef | 0.0000 |
| Skin | 0.0149 | 0.1693 | 0.0881 | 11.3508 |
| Hepatic | 0.0149 | 0.0129 | 1.1477 | 0.8713 |
| Heart | 0.0381 | 0.0412 | 0.9249 | 1.0812 |
| Testicles | 0.0183 | 0.0000 | undef | 0.0000 |
| Lungs | 0.0174 | 0.0615 | 0.2836 | 3.5259 |
| Stomach-esophagus | 0.0097 | 0.0383 | 0.2520 | 3.9685 |
| Muscle-skeleton | 0.0171 | 0.0180 | 0.9517 | 1.0508 |
| Kidneys | 0.0208 | 0.0548 | 0.3799 | 2.6323 |
| Pancreas | 0.0284 | 0.0000 | undef | 0.0000 |
| Penis | 0.0180 | 0.1066 | 0.1685 | 5.9360 |
| Prostate | 0.0119 | 0.0213 | 0.5593 | 1.7879 |
| Uterus | 0.0116 | 0.0285 | 0.4061 | 2.4622 |
| Breast hyperplasia | 0.0109 | | | |
| Small intestine | 0.0374 | | | |
| Prostatic hyperplasia | 0.0386 | | | |
| Seminal vesicles | 0.0356 | | | |
| Sensory organs | 0.0000 | | | |
| White blood cells | 0.0113 | | | |
| | FETUS | | | |
| Development | 0.0307 | | | |
| Gastrointestinal | 0.0216 | | | |
| Brain | 0.0188 | | | |
| Hematopoietic | 0.0079 | | | |
| Heart-blood vessels | 0.0245 | | | |
| Lungs | 0.0259 | | | |
| Kidneys | 0.0062 | | | |
| Prostate | 0.0748 | | | |
| Sensory organs | 0.0000 | | | |
| | STANDARDIZED/ | | | |
| | SUBTRACTED | | | |
| | LIBRARIES | | | |
| Breast | 0.0068 | | | |
| Ovary-uterus | 0.0205 | | | |
| Endocrine tissue | 0.0000 | | | |
| Fetal | 0.0233 | | | |
| Gastrointestinal | 0.0366 | | | |
| Hematopoietic | 0.0000 | | | |
| Skin-muscle | 0.0615 | | | |
| Testicles | 0.0078 | | | |
| Lungs | 0.0164 | | | |
| Nerves | 0.0181 | | | |
| Prostate | 0.0192 | | | |
| Sensory organs | 0.0387 | | | |

Electronic Northern for SEQ. ID NO.: 9

| | | | | |
|---|---|---|---|---|
| | NORMAL | | | |
| Bladder | 0.0046 | 0.0128 | 0.3637 | 2.7495 |
| Breast | 0.0053 | 0.0218 | 0.2446 | 4.0878 |
| Ovary | 0.0122 | 0.0026 | 4.6745 | 0.2139 |
| Endocrine tissue | 0.0036 | 0.0109 | 0.3349 | 2.9861 |
| Gastrointestinal | 0.0213 | 0.0048 | 4.4784 | 0.2233 |
| Brain | 0.0051 | 0.0000 | undef | 0.0000 |
| Hematopoietic | 0.0028 | 0.0000 | undef | 0.0000 |
| Skin | 0.0348 | 0.0000 | undef | 0.0000 |
| Hepatic | 0.0297 | 0.0000 | undef | 0.0000 |
| Heart | 0.0064 | 0.0137 | 0.4624 | 2.1624 |
| Testicles | 0.0000 | 0.0000 | undef | undef |
| Lungs | 0.0324 | 0.0189 | 1.7118 | 0.5842 |
| Stomach-esophagus | 0.0000 | 0.0077 | 0.0000 | undef |
| Muscle-skeleton | 0.0017 | 0.0000 | undef | 0.0000 |
| Kidneys | 0.0357 | 0.0000 | undef | 0.0000 |
| Pancreas | 0.0208 | 0.0000 | undef | 0.0000 |
| Penis | 0.0000 | 0.0000 | undef | undef |
| Prostate | 0.0214 | 0.0085 | 2.5169 | 0.3973 |
| Uterus | 0.0050 | 0.0000 | undef | 0.0000 |
| Breast hyperplasia | 0.0145 | | | |
| Small intestine | 0.0031 | | | |
| Prostatic hyperplasia | 0.0178 | | | |
| Seminal vesicles | 0.0089 | | | |
| Sensory organs | 0.0000 | | | |
| White blood cells | 0.0000 | | | |
| | FETUS | | | |
| Development | 0.0000 | | | |
| Gastrointestinal | 0.0062 | | | |
| Brain | 0.0000 | | | |
| Hematopoietic | 0.0039 | | | |
| Heart-blood vessels | 0.0041 | | | |
| Lungs | 0.0148 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0068 | | | |
| Endocrine tissue | 0.0000 | | | |
| Fetal | 0.0058 | | | |
| Gastrointestinal | 0.0244 | | | |
| Hematopoietic | 0.0000 | | | |
| Skin-muscle | 0.0130 | | | |
| Testicles | 0.0000 | | | |
| Lungs | 0.0246 | | | |
| Nerves | 0.0020 | | | |
| Prostate | 0.0256 | | | |
| Sensory organs | 0.0000 | | | |

Electronic Northern for SEQ. ID NO.: 11

| | | | | |
|---|---|---|---|---|
| | NORMAL | | | |
| Bladder | 0.0139 | 0.0179 | 0.7793 | 1.2831 |
| Breast | 0.0133 | 0.0414 | 0.3219 | 3.1067 |
| Ovary | 0.0000 | 0.0156 | 0.0000 | undef |
| Endocrine tissue | 0.0073 | 0.0027 | 2.6791 | 0.3733 |
| Gastrointestinal | 0.0058 | 0.0048 | 1.2214 | 0.8187 |
| Brain | 0.0093 | 0.0153 | 0.6082 | 1.6441 |
| Hematopoietic | 0.0098 | 0.0000 | undef | 0.0000 |
| Skin | 0.0199 | 0.0000 | undef | 0.0000 |
| Hepatic | 0.0050 | 0.0129 | 0.3826 | 2.6139 |
| Heart | 0.0085 | 0.0137 | 0.6166 | 1.6218 |
| Testicles | 0.0061 | 0.0117 | 0.5224 | 1.9144 |
| Lungs | 0.0237 | 0.0213 | 1.1120 | 0.8993 |
| Stomach-esophagus | 0.0097 | 0.0077 | 1.2599 | 0.7937 |
| Muscle-skeleton | 0.0103 | 0.0180 | 0.5710 | 1.7513 |
| Kidneys | 0.0030 | 0.0479 | 0.0620 | 16.1231 |
| Pancreas | 0.0038 | 0.0221 | 0.1714 | 5.8337 |
| Penis | 0.0090 | 0.0533 | 0.1685 | 5.9360 |
| Prostate | 0.0071 | 0.0085 | 0.8390 | 1.1919 |
| Uterus | 0.0050 | 0.0214 | 0.2321 | 4.3088 |
| Breast hyperplasia | 0.0182 | | | |
| Small intestine | 0.0062 | | | |
| Prostatic hyperplasia | 0.0000 | | | |
| Seminal vesicles | 0.0089 | | | |
| Sensory organs | 0.0000 | | | |
| White blood cells | 0.0331 | | | |
| | FETUS | | | |
| Development | 0.0000 | | | |
| Gastrointestinal | 0.0031 | | | |
| Brain | 0.0000 | | | |
| Hematopoietic | 0.0000 | | | |
| Heart-blood vessels | 0.0000 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0340 | | | |
| Ovary-uterus | 0.0023 | | | |
| Endocrine tissue | 0.0000 | | | |
| Fetal | 0.0006 | | | |
| Gastrointestinal | 0.0000 | | | |
| Hematopoietic | 0.0057 | | | |
| Skin-muscle | 0.0032 | | | |
| Testicles | 0.0000 | | | |
| Lungs | 0.0656 | | | |
| Nerves | 0.0030 | | | |
| Prostate | 0.0000 | | | |
| Sensory organs | 0.0387 | | | |

Electronic Northern for SEQ. ID NO.: 12

| | | | | |
|---|---|---|---|---|
| | NORMAL | | | |
| Bladder | 0.0093 | 0.0026 | 3.6370 | 0.2750 |
| Breast | 0.0160 | 0.0545 | 0.2936 | 3.4065 |
| Ovary | 0.0061 | 0.0078 | 0.7791 | 1.2836 |
| Endocrine tissue | 0.0091 | 0.0082 | 1.1163 | 0.8958 |
| Gastrointestinal | 0.0019 | 0.0000 | undef | 0.0000 |
| Brain | 0.0034 | 0.0000 | undef | 0.0000 |
| Hematopoietic | 0.0014 | 0.0000 | undef | 0.0000 |
| Skin | 0.0149 | 0.0000 | undef | 0.0000 |
| Hepatic | 0.0000 | 0.0000 | undef | undef |
| Heart | 0.0021 | 0.0275 | 0.0771 | 12.9744 |
| Testicles | 0.0061 | 0.0000 | undef | 0.0000 |
| Lungs | 0.0025 | 0.0000 | undef | 0.0000 |
| Stomach-esophagus | 0.0000 | 0.0000 | undef | undef |
| Muscle-skeleton | 0.0034 | 0.0000 | undef | 0.0000 |
| Kidneys | 0.0089 | 0.0000 | undef | 0.0000 |
| Pancreas | 0.0019 | 0.0000 | undef | 0.0000 |
| Penis | 0.0120 | 0.0000 | undef | 0.0000 |
| Prostate | 0.0048 | 0.0106 | 0.4475 | 2.2349 |
| Uterus | 0.0066 | 0.0000 | undef | 0.0000 |
| Breast hyperplasia | 0.0218 | | | |
| Small intestine | 0.0000 | | | |
| Prostatic hyperplasia | 0.0238 | | | |
| Seminal vesicles | 0.0000 | | | |
| Sensory organs | 0.0235 | | | |
| White blood cells | 0.0044 | | | |
| | FETUS | | | |
| Development | 0.0000 | | | |
| Gastrointestinal | 0.0031 | | | |
| Brain | 0.0125 | | | |
| Hematopoietic | 0.0039 | | | |
| Heart-blood vessels | 0.0000 | | | |
| Lungs | 0.0074 | | | |
| Kidneys | 0.0000 | | | |
| Prostate | 0.0249 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0046 | | | |
| Endocrine tissue | 0.0000 | | | |
| Fetal | 0.0041 | | | |
| Gastrointestinal | 0.0000 | | | |
| Hematopoietic | 0.0000 | | | |
| Skin-muscle | 0.0000 | | | |
| Testicles | 0.0000 | | | |
| Lungs | 0.0000 | | | |
| Nerves | 0.0070 | | | |
| Prostate | 0.0128 | | | |
| Sensory organs | 0.0000 | | | |

Electronic Northern for SEQ. ID NO.: 13

| | | | | |
|---|---|---|---|---|
| | NORMAL | | | |
| Bladder | 0.0186 | 0.0128 | 1.4548 | 0.6874 |
| Breast | 0.0093 | 0.0283 | 0.3293 | 3.0366 |
| Ovary | 0.0122 | 0.0026 | 4.6745 | 0.2139 |
| Endocrine tissue | 0.0237 | 0.0136 | 1.7414 | 0.5742 |
| Gastrointestinal | 0.0271 | 0.0000 | undef | 0.0000 |
| Brain | 0.0068 | 0.0120 | 0.5630 | 1.7762 |
| Hematopoietic | 0.0084 | 0.0000 | undef | 0.0000 |
| Skin | 0.0050 | 0.0000 | undef | 0.0000 |
| Hepatic | 0.0198 | 0.0065 | 3.0606 | 0.3267 |
| Heart | 0.0307 | 0.0275 | 1.1176 | 0.8948 |
| Testicles | 0.0122 | 0.0234 | 0.5224 | 1.9144 |
| Lungs | 0.0125 | 0.0047 | 2.6336 | 0.3797 |
| Stomach-esophagus | 0.0290 | 0.0230 | 1.2599 | 0.7937 |
| Muscle-skeleton | 0.0086 | 0.0180 | 0.4758 | 2.1015 |
| Kidneys | 0.0119 | 0.0137 | 0.8683 | 1.1517 |
| Pancreas | 0.0038 | 0.0166 | 0.2286 | 4.3753 |
| Penis | 0.0539 | 0.0000 | undef | 0.0000 |
| Prostate | 0.0214 | 0.0341 | 0.6292 | 1.5892 |
| Uterus | 0.0446 | 0.0142 | 3.1331 | 0.3192 |
| Breast hyperplasia | 0.0036 | | | |
| Small intestine | 0.0093 | | | |
| Prostatic hyperplasia | 0.0149 | | | |
| Seminal vesicles | 0.0534 | | | |
| Sensory organs | 0.0000 | | | |
| White blood cells | 0.0104 | | | |
| | FETUS | | | |
| Development | 0.0307 | | | |
| Gastrointestinal | 0.0308 | | | |
| Brain | 0.0063 | | | |
| Hematopoietic | 0.0039 | | | |
| Heart-blood vessels | 0.0082 | | | |
| Lungs | 0.0370 | | | |
| Kidneys | 0.0062 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0204 | | | |
| Ovary-uterus | 0.0114 | | | |
| Endocrine tissue | 0.0245 | | | |
| Fetal | 0.0099 | | | |
| Gastrointestinal | 0.0000 | | | |
| Hematopoietic | 0.0285 | | | |
| Skin-muscle | 0.0130 | | | |
| Testicles | 0.0000 | | | |
| Lungs | 0.0082 | | | |
| Nerves | 0.0110 | | | |
| Prostate | 0.0128 | | | |
| Sensory organs | 0.0155 | | | |

Electronic Northern for SEQ. ID NO.: 14

| | | | | |
|---|---|---|---|---|
| | NORMAL | | | |
| Bladder | 0.0000 | 0.0077 | 0.0000 | undef |
| Breast | 0.0080 | 0.0261 | 0.3058 | 3.2702 |
| Ovary | 0.0061 | 0.0234 | 0.2597 | 3.8507 |
| Endocrine tissue | 0.0073 | 0.0163 | 0.4465 | 2.2395 |
| Gastrointestinal | 0.0078 | 0.0048 | 1.6285 | 0.6141 |
| Brain | 0.0076 | 0.0077 | 0.9953 | 1.0047 |
| Hematopoietic | 0.0182 | 0.0000 | undef | 0.0000 |
| Skin | 0.0000 | 0.0000 | undef | undef |
| Hepatic | 0.0000 | 0.0388 | 0.0000 | undef |
| Heart | 0.0042 | 0.0000 | undef | 0.0000 |
| Testicles | 0.0061 | 0.0000 | undef | 0.0000 |
| Lungs | 0.0125 | 0.0165 | 0.7524 | 1.3290 |
| Stomach-esophagus | 0.0097 | 0.0153 | 0.6300 | 1.5874 |
| Muscle-skeleton | 0.0034 | 0.0000 | undef | 0.0000 |
| Kidneys | 0.0149 | 0.0137 | 1.0854 | 0.9213 |
| Pancreas | 0.0019 | 0.0055 | 0.3428 | 2.9168 |
| Penis | 0.0090 | 0.0267 | 0.3369 | 2.9680 |
| Prostate | 0.0524 | 0.0341 | 1.5381 | 0.6501 |
| Uterus | 0.0066 | 0.0000 | undef | 0.0000 |
| Breast hyperplasia | 0.0036 | | | |
| Small intestine | 0.0062 | | | |
| Prostatic hyperplasia | 0.0386 | | | |
| Seminal vesicles | 0.0000 | | | |
| Sensory organs | 0.0235 | | | |
| White blood cells | 0.0113 | | | |
| | FETUS | | | |
| Development | 0.0154 | | | |
| Gastrointestinal | 0.0092 | | | |
| Brain | 0.0000 | | | |
| Hematopoietic | 0.0000 | | | |
| Heart-blood vessels | 0.0041 | | | |
| Lungs | 0.0259 | | | |
| Kidneys | 0.0247 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0183 | | | |
| Endocrine tissue | 0.0000 | | | |
| Fetal | 0.0181 | | | |
| Gastrointestinal | 0.0366 | | | |
| Hematopoietic | 0.0285 | | | |
| Skin-muscle | 0.0324 | | | |
| Testicles | 0.0078 | | | |
| Lungs | 0.0246 | | | |
| Nerves | 0.0020 | | | |
| Prostate | 0.0705 | | | |

Electronic Northern for SEQ. ID NO.: 15

| | | | | |
|---|---|---|---|---|
| | NORMAL | | | |
| Bladder | 0.0093 | 0.0102 | 0.9092 | 1.0998 |
| Breast | 0.0027 | 0.0196 | 0.1359 | 7.3580 |
| Ovary | 0.0000 | 0.0234 | 0.0000 | undef |
| Endocrine tissue | 0.0055 | 0.0381 | 0.1435 | 6.9675 |
| Gastrointestinal | 0.0058 | 0.0143 | 0.4071 | 2.4562 |
| Brain | 0.0076 | 0.0066 | 1.1612 | 0.8612 |
| Hematopoietic | 0.0084 | 0.0000 | undef | 0.0000 |
| Skin | 0.0000 | 0.0000 | undef | undef |
| Hepatic | 0.0000 | 0.0000 | undef | undef |
| Heart | 0.0201 | 0.0000 | undef | 0.0000 |
| Testicles | 0.0122 | 0.0000 | undef | 0.0000 |
| Lungs | 0.0100 | 0.0165 | 0.6020 | 1.6612 |
| Stomach-esophagus | 0.0000 | 0.0153 | 0.0000 | undef |
| Muscle-skeleton | 0.0017 | 0.0060 | 0.2855 | 3.5025 |
| Kidneys | 0.0119 | 0.0137 | 0.8683 | 1.1517 |
| Pancreas | 0.0038 | 0.0000 | undef | 0.0000 |
| Penis | 0.0120 | 0.0000 | undef | 0.0000 |
| Prostate | 0.0095 | 0.0128 | 0.7458 | 1.3409 |
| Uterus | 0.0066 | 0.0142 | 0.4642 | 2.1544 |
| Breast hyperplasia | 0.0036 | | | |
| Small intestine | 0.0187 | | | |
| Prostatic hyperplasia | 0.0089 | | | |
| Seminal vesicles | 0.0356 | | | |
| Sensory organs | 0.0118 | | | |
| White blood cells | 0.0052 | | | |
| | FETUS | | | |
| Development | 0.0000 | | | |
| Gastrointestinal | 0.0123 | | | |
| Brain | 0.0188 | | | |
| Hematopoietic | 0.0118 | | | |
| Heart-blood vessels | 0.0123 | | | |
| Lungs | 0.0185 | | | |
| Kidneys | 0.0124 | | | |
| Prostate | 0.0000 | | | |
| Sensory organs | 0.0140 | | | |
| | STANDARDIZED/ | | | |
| | SUBTRACTED | | | |
| | LIBRARIES | | | |
| Breast | 0.0000 | | | |
| Ovary-uterus | 0.0068 | | | |
| Endocrine tissue | 0.0000 | | | |
| Fetal | 0.0064 | | | |
| Gastrointestinal | 0.0122 | | | |
| Hematopoietic | 0.0057 | | | |
| Skin-muscle | 0.0130 | | | |
| Testicles | 0.0312 | | | |
| Lungs | 0.0164 | | | |
| Nerves | 0.0070 | | | |
| Prostate | 0.0128 | | | |
| Sensory organs | 0.0000 | | | |

Electronic Northern for SEQ. ID NO.: 17

| | | | | |
|---|---|---|---|---|
| | NORMAL | | | |
| Bladder | 0.0046 | 0.0077 | 0.6062 | 1.6497 |
| Breast | 0.0040 | 0.0240 | 0.1668 | 5.9954 |
| Ovary | 0.0030 | 0.0130 | 0.2337 | 4.2786 |
| Endocrine tissue | 0.0018 | 0.0000 | undef | 0.0000 |
| Gastrointestinal | 0.0271 | 0.0095 | 2.8499 | 0.3509 |
| Brain | 0.0000 | 0.0000 | undef | undef |
| Hematopoietic | 0.0042 | 0.0000 | undef | 0.0000 |
| Skin | 0.0448 | 0.0000 | undef | 0.0000 |
| Hepatic | 0.0000 | 0.0000 | undef | undef |
| Heart | 0.0000 | 0.0000 | undef | undef |
| Testicles | 0.0000 | 0.0234 | 0.0000 | undef |
| Lungs | 0.0087 | 0.0071 | 1.2290 | 0.8137 |
| Stomach-esophagus | 0.0869 | 0.0230 | 3.7798 | 0.2646 |
| Muscle-skeleton | 0.0000 | 0.0000 | undef | undef |
| Kidneys | 0.0000 | 0.0000 | undef | undef |
| Pancreas | 0.0000 | 0.0000 | undef | undef |
| Penis | 0.0180 | 0.0533 | 0.3369 | 2.9680 |
| Prostate | 0.0048 | 0.0021 | 2.2373 | 0.4470 |
| Uterus | 0.0116 | 0.0000 | undef | 0.0000 |
| Breast hyperplasia | 0.0000 | | | |
| Small intestine | 0.0125 | | | |
| Prostatic hyperplasia | 0.0000 | | | |
| Seminal vesicles | 0.0089 | | | |
| Sensory organs | 0.0118 | | | |
| While blood cells | 0.0000 | | | |
| | FETUS | | | |
| Development | 0.0000 | | | |
| Gastrointestinal | 0.0000 | | | |
| Brain | 0.0000 | | | |
| Hematopoietic | 0.0039 | | | |
| Heart-blood vessels | 0.0000 | | | |
| Lungs | 0.0074 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0000 | | | |
| Endocrine tissue | 0.0000 | | | |
| Fetal | 0.0000 | | | |
| Gastrointestinal | 0.0000 | | | |
| Hematopoietic | 0.0057 | | | |
| Skin-muscle | 0.0000 | | | |
| Testicles | 0.0000 | | | |
| Lungs | 0.0000 | | | |
| Nerves | 0.0000 | | | |
| Prostate | 0.0000 | | | |
| Sensory organs | 0.0000 | | | |

Electronic Northern for SEQ. ID NO.: 18

| | | | | |
|---|---|---|---|---|
| | NORMAL | | | |
| Bladder | 0.0000 | 0.0077 | 0.0000 | undef |
| Breast | 0.0027 | 0.0196 | 0.1359 | 7.3580 |
| Ovary | 0.0030 | 0.0208 | 0.1461 | 6.8457 |
| Endocrine tissue | 0.0182 | 0.0109 | 1.6745 | 0.5972 |
| Gastrointestinal | 0.0019 | 0.0000 | undef | 0.0000 |
| Brain | 0.0051 | 0.0099 | 0.5161 | 1.9377 |
| Hematopoietic | 0.0028 | 0.0000 | undef | 0.0000 |
| Skin | 0.0000 | 0.0000 | undef | undef |
| Hepatic | 0.0050 | 0.0065 | 0.7651 | 1.3069 |
| Heart | 0.0085 | 0.0000 | undef | 0.0000 |
| Testicles | 0.0000 | 0.0117 | 0.0000 | undef |
| Lungs | 0.0100 | 0.0071 | 1.4046 | 0.7120 |
| Stomach-esophagus | 0.0097 | 0.0000 | undef | 0.0000 |
| Muscle-skeleton | 0.0188 | 0.0060 | 3.1406 | 0.3184 |
| Kidneys | 0.0030 | 0.0000 | undef | 0.0000 |
| Pancreas | 0.0000 | 0.0055 | 0.0000 | undef |
| Penis | 0.0090 | 0.0533 | 0.1685 | 5.9360 |
| Prostate | 0.0191 | 0.0106 | 1.7898 | 0.5587 |
| Uterus | 0.0116 | 0.0071 | 1.6246 | 0.6155 |
| Breast hyperplasia | 0.0036 | | | |
| Small intestine | 0.0062 | | | |
| Prostatic hyperplasia | 0.0208 | | | |
| Seminal vesicles | 0.0089 | | | |
| Sensory organs | 0.0000 | | | |
| White blood cells | 0.0044 | | | |
| | FETUS | | | |
| Development | 0.0154 | | | |
| Gastrointestinal | 0.0062 | | | |
| Brain | 0.0000 | | | |
| Hematopoietic | 0.0118 | | | |
| Heart-blood vessels | 0.0245 | | | |
| Lungs | 0.0074 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0136 | | | |
| Ovary-uterus | 0.0000 | | | |
| Endocrine tissue | 0.0000 | | | |
| Fetal | 0.0082 | | | |
| Gastrointestinal | 0.0122 | | | |
| Hematopoietic | 0.0456 | | | |
| Skin-muscle | 0.0097 | | | |
| Testicles | 0.0078 | | | |
| Lungs | 0.0164 | | | |
| Nerves | 0.0050 | | | |
| Prostate | 0.0064 | | | |
| Sensory organs | 0.0000 | | | |

Electronic Northern for SEQ. ID NO.: 19

| | | | | |
|---|---|---|---|---|
| | NORMAL | | | |
| Bladder | 0.0000 | 0.0000 | undef | undef |
| Breast | 0.0120 | 0.0763 | 0.1573 | 6.3588 |
| Ovary | 0.0608 | 0.0234 | 2.5969 | 0.3851 |
| Endocrine tissue | 0.0073 | 0.0245 | 0.2977 | 3.3593 |
| Gastrointestinal | 0.0872 | 0.1000 | 0.8724 | 1.1462 |
| Brain | 0.0017 | 0.0000 | undef | 0.0000 |
| Hematopoietic | 0.0014 | 0.0000 | undef | 0.0000 |
| Skin | 0.0000 | 0.0000 | undef | undef |
| Hepatic | 0.0149 | 0.0388 | 0.3826 | 2.6139 |
| Heart | 0.0011 | 0.0000 | undef | 0.0000 |
| Testicles | 0.0000 | 0.0000 | undef | undef |
| Lungs | 0.0050 | 0.0355 | 0.1405 | 7.1196 |
| Stomach-esophagus | 0.0000 | 0.0230 | 0.0000 | undef |
| Muscle-skeleton | 0.0000 | 0.0000 | undef | undef |
| Kidneys | 0.0000 | 0.0000 | undef | undef |
| Pancreas | 0.0000 | 0.0166 | 0.0000 | undef |
| Penis | 0.0000 | 0.0000 | undef | undef |
| Prostate | 0.0119 | 0.0958 | 0.1243 | 8.0455 |
| Uterus | 0.0017 | 0.0214 | 0.0774 | 12.9263 |
| Breast hyperplasia | 0.0073 | | | |
| Small intestine | 0.0436 | | | |
| Prostatic hyperplasia | 0.0119 | | | |
| Seminal vesicles | 0.0089 | | | |
| Sensory organs | 0.0470 | | | |
| White blood cells | 0.0009 | | | |
| | FETUS | | | |
| Development | 0.0000 | | | |
| Gastrointestinal | 0.0247 | | | |
| Brain | 0.0000 | | | |
| Hematopoietic | 0.0000 | | | |
| Heart-blood vessels | 0.0000 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0205 | | | |
| Endocrine tissue | 0.0000 | | | |
| Fetal | 0.0052 | | | |
| Gastrointestinal | 0.0366 | | | |
| Hematopoietic | 0.0000 | | | |
| Skin-muscle | 0.0000 | | | |
| Testicles | 0.0000 | | | |
| Lungs | 0.0000 | | | |
| Nerves | 0.0000 | | | |
| Prostate | 0.0321 | | | |
| Sensory organs | 0.0000 | | | |

Electronic Northern for SEQ. ID NO.: 20

| | | | | |
|---|---|---|---|---|
| | NORMAL | | | |
| Bladder | 0.0000 | 0.0051 | 0.0000 | undef |
| Breast | 0.0040 | 0.0240 | 0.1668 | 5.9954 |
| Ovary | 0.0182 | 0.0078 | 2.3372 | 0.4279 |
| Endocrine tissue | 0.0164 | 0.0245 | 0.6698 | 1.4930 |
| Gastrointestinal | 0.0213 | 0.0190 | 1.1196 | 0.8932 |
| Brain | 0.0144 | 0.0186 | 0.7741 | 1.2918 |
| Hematopoietic | 0.0056 | 0.0000 | undef | 0.0000 |
| Skin | 0.0000 | 0.0000 | undef | undef |
| Hepatic | 0.0248 | 0.0065 | 3.8257 | 0.2614 |
| Heart | 0.0138 | 0.0000 | undef | 0.0000 |
| Testicles | 0.0428 | 0.0000 | undef | 0.0000 |
| Lungs | 0.0199 | 0.0165 | 1.2039 | 0.8306 |
| Stomach-esophagus | 0.0097 | 0.0230 | 0.4200 | 2.3811 |
| Muscle-skeleton | 0.0120 | 0.0000 | undef | 0.0000 |
| Kidneys | 0.0119 | 0.0137 | 0.8683 | 1.1517 |
| Pancreas | 0.0038 | 0.0000 | undef | 0.0000 |
| Penis | 0.0150 | 0.0000 | undef | 0.0000 |
| Prostate | 0.0119 | 0.0192 | 0.6215 | 1.6091 |
| Uterus | 0.0132 | 0.0142 | 0.9283 | 1.0772 |
| Breast hyperplasia | 0.0254 | | | |
| Small intestine | 0.0093 | | | |
| Prostatic hyperplasia | 0.0238 | | | |
| Seminal vesicles | 0.0178 | | | |
| Sensory organs | 0.0118 | | | |
| White blood cells | 0.0044 | | | |
| | FETUS | | | |
| Development | 0.0000 | | | |
| Gastrointestinal | 0.0062 | | | |
| Brain | 0.0125 | | | |
| Hematopoietic | 0.0000 | | | |
| Heart-blood vessels | 0.0000 | | | |
| Lungs | 0.0111 | | | |
| Kidneys | 0.0185 | | | |
| Prostate | 0.0000 | | | |
| Sensory organs | 0.0140 | | | |
| | STANDARDIZED/ | | | |
| | SUBTRACTED | | | |
| | LIBRARIES | | | |
| Breast | 0.0000 | | | |
| Ovary-uterus | 0.0068 | | | |
| Endocrine tissue | 0.0000 | | | |
| Fetal | 0.0058 | | | |
| Gastrointestinal | 0.0488 | | | |
| Hematopoietic | 0.0114 | | | |
| Skin-muscle | 0.0065 | | | |
| Testicles | 0.0000 | | | |
| Lungs | 0.0000 | | | |
| Nerves | 0.0040 | | | |
| Prostate | 0.0385 | | | |
| Sensory organs | 0.0000 | | | |

Electronic Northern for SEQ. ID NO.: 21

| | | | | |
|---|---|---|---|---|
| | NORMAL | | | |
| Bladder | 0.0000 | 0.0026 | 0.0000 | undef |
| Breast | 0.0067 | 0.0261 | 0.2548 | 3.9243 |
| Ovary | 0.0000 | 0.0000 | undef | undef |
| Endocrine tissue | 0.0018 | 0.0000 | undef | 0.0000 |
| Gastrointestinal | 0.0039 | 0.0048 | 0.8143 | 1.2281 |
| Brain | 0.0017 | 0.0022 | 0.7741 | 1.2918 |
| Hematopoietic | 0.0112 | 0.0000 | undef | 0.0000 |
| Skin | 0.0050 | 0.0000 | undef | 0.0000 |
| Hepatic | 0.0000 | 0.0065 | 0.0000 | undef |
| Heart | 0.0042 | 0.0137 | 0.3083 | 3.2436 |
| Testicles | 0.0000 | 0.0000 | undef | undef |
| Lungs | 0.0037 | 0.0024 | 1.5801 | 0.6329 |
| Stomach-esophagus | 0.0000 | 0.0000 | undef | undef |
| Muscle-skeleton | 0.0034 | 0.0000 | undef | 0.0000 |
| Kidneys | 0.0030 | 0.0137 | 0.2171 | 4.6066 |
| Pancreas | 0.0000 | 0.0055 | 0.0000 | undef |
| Penis | 0.0060 | 0.0000 | undef | 0.0000 |
| Prostate | 0.0024 | 0.0000 | undef | 0.0000 |
| Uterus | 0.0017 | 0.0000 | undef | 0.0000 |
| Breast hyperplasia | 0.0073 | | | |
| Small intestine | 0.0000 | | | |
| Prostatic hyperplasia | 0.0000 | | | |
| Seminal vesicles | 0.0000 | | | |
| Sensory organs | 0.0000 | | | |
| White blood cells | 0.0052 | | | |
| | FETUS | | | |
| Development | 0.0000 | | | |
| Gastrointestinal | 0.0000 | | | |
| Brain | 0.0000 | | | |
| Hematopoietic | 0.0039 | | | |
| Heart-blood vessels | 0.0000 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0068 | | | |
| Ovary-uterus | 0.0046 | | | |
| Endocrine tissue | 0.0000 | | | |
| Fetal | 0.0023 | | | |
| Gastrointestinal | 0.0000 | | | |
| Hematopoietic | 0.0228 | | | |
| Skin-muscle | 0.0000 | | | |
| Testicles | 0.0000 | | | |
| Lungs | 0.0000 | | | |
| Nerves | 0.0000 | | | |
| Prostate | 0.0064 | | | |
| Sensory organs | 0.0000 | | | |

Electronic Northern for SEQ. ID NO.: 22

| | | | | |
|---|---|---|---|---|
| | NORMAL | | | |
| Bladder | 0.0046 | 0.0051 | 0.9092 | 1.0998 |
| Breast | 0.0093 | 0.0218 | 0.4281 | 2.3359 |
| Ovary | 0.0061 | 0.0078 | 0.7791 | 1.2836 |
| Endocrine tissue | 0.0055 | 0.0000 | undef | 0.0000 |
| Gastrointestinal | 0.0039 | 0.0000 | undef | 0.0000 |
| Brain | 0.0059 | 0.0033 | 1.8062 | 0.5536 |
| Hematopoietic | 0.0126 | 0.0000 | undef | 0.0000 |
| Skin | 0.0050 | 0.0847 | 0.0587 | 17.0262 |
| Hepatic | 0.0050 | 0.0000 | undef | 0.0000 |
| Heart | 0.0138 | 0.0137 | 1.0020 | 0.9980 |
| Testicles | 0.0061 | 0.0000 | undef | 0.0000 |
| Lungs | 0.0112 | 0.0024 | 4.7404 | 0.2110 |
| Stomach-esophagus | 0.0097 | 0.0537 | 0.1800 | 5.5559 |
| Muscle-skeleton | 0.0086 | 0.0180 | 0.4758 | 2.1015 |
| Kidneys | 0.0119 | 0.0274 | 0.4342 | 2.3033 |
| Pancreas | 0.0057 | 0.0000 | undef | 0.0000 |
| Penis | 0.0030 | 0.0267 | 0.1123 | 8.9040 |
| Prostate | 0.0143 | 0.0043 | 3.3559 | 0.2980 |
| Uterus | 0.0033 | 0.0071 | 0.4642 | 2.1544 |
| Breast hyperplasia | 0.0036 | | | |
| Small intestine | 0.0062 | | | |
| Prostatic hyperplasia | 0.0089 | | | |
| Seminal vesicles | 0.0089 | | | |
| Sensory organs | 0.0000 | | | |
| White blood cells | 0.0070 | | | |
| | FETUS | | | |
| Development | 0.0000 | | | |
| Gastrointestinal | 0.0092 | | | |
| Brain | 0.0125 | | | |
| Hematopoietic | 0.0157 | | | |
| Heart-blood vessels | 0.0041 | | | |
| Lungs | 0.0037 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0114 | | | |
| Endocrine tissue | 0.0000 | | | |
| Fetal | 0.0029 | | | |
| Gastrointestinal | 0.0000 | | | |
| Hematopoietic | 0.0114 | | | |
| Skin-muscle | 0.0000 | | | |
| Testicles | 0.0000 | | | |
| Lungs | 0.0000 | | | |
| Nerves | 0.0020 | | | |
| Prostate | 0.0000 | | | |
| Sensory organs | 0.0000 | | | |

Electronic Northern for SEQ. ID NO.: 23

| | | | | |
|---|---|---|---|---|
| | NORMAL | | | |
| Bladder | 0.0000 | 0.0077 | 0.0000 | undef |
| Breast | 0.0040 | 0.0131 | 0.3058 | 3.2702 |
| Ovary | 0.0030 | 0.0000 | undef | 0.0000 |
| Endocrine tissue | 0.0109 | 0.0000 | undef | 0.0000 |
| Gastrointestinal | 0.0136 | 0.0048 | 2.8499 | 0.3509 |
| Brain | 0.0059 | 0.0099 | 0.6021 | 1.6609 |
| Hematopoietic | 0.0056 | 0.0000 | undef | 0.0000 |
| Skin | 0.0597 | 0.0000 | undef | 0.0000 |
| Hepatic | 0.0000 | 0.0065 | 0.0000 | undef |
| Heart | 0.0064 | 0.0137 | 0.4624 | 2.1624 |
| Testicles | 0.0000 | 0.0351 | 0.0000 | undef |
| Lungs | 0.0012 | 0.0095 | 0.1317 | 7.5943 |
| Stomach-esophagus | 0.0193 | 0.0230 | 0.8399 | 1.1905 |
| Muscle-skeleton | 0.0069 | 0.0060 | 1.1420 | 0.8756 |
| Kidneys | 0.0059 | 0.0068 | 0.8683 | 1.1517 |
| Pancreas | 0.0038 | 0.0000 | undef | 0.0000 |
| Penis | 0.0000 | 0.0000 | undef | undef |
| Prostate | 0.0143 | 0.0213 | 0.6712 | 1.4899 |
| Uterus | 0.0066 | 0.0000 | undef | 0.0000 |
| Breast hyperplasia | 0.0000 | | | |
| Small intestine | 0.0093 | | | |
| Prostatic hyperplasia | 0.0089 | | | |
| Seminal vesicles | 0.0178 | | | |
| Sensory organs | 0.0118 | | | |
| White blood cells | 0.0044 | | | |
| | FETUS | | | |
| Development | 0.0154 | | | |
| Gastrointestinal | 0.0031 | | | |
| Brain | 0.0250 | | | |
| Hematopoietic | 0.0000 | | | |
| Heart-blood vessels | 0.0245 | | | |
| Lungs | 0.0037 | | | |
| Kidneys | 0.0185 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0136 | | | |
| Ovary-uterus | 0.0297 | | | |
| Endocrine tissue | 0.0000 | | | |
| Fetal | 0.0222 | | | |
| Gastrointestinal | 0.0000 | | | |
| Hematopoietic | 0.0000 | | | |
| Skin-muscle | 0.0000 | | | |
| Testicles | 0.0000 | | | |
| Lungs | 0.0082 | | | |
| Nerves | 0.0030 | | | |
| Prostate | 0.0064 | | | |
| Sensory organs | 0.0077 | | | |

Electronic Northern for SEQ. ID NO.: 24

| | | | | |
|---|---|---|---|---|
| | NORMAL | | | |
| Bladder | 0.0046 | 0.0102 | 0.4546 | 2.1996 |
| Breast | 0.0027 | 0.0174 | 0.1529 | 6.5404 |
| Ovary | 0.0152 | 0.0234 | 0.6492 | 1.5403 |
| Endocrine tissue | 0.0146 | 0.0327 | 0.4465 | 2.2395 |
| Gastrointestinal | 0.0291 | 0.0095 | 3.0535 | 0.3275 |
| Brain | 0.0203 | 0.0252 | 0.8078 | 1.2380 |
| Hematopoietic | 0.0084 | 0.0000 | undef | 0.0000 |
| Skin | 0.0149 | 0.0000 | undef | 0.0000 |
| Lungs | 0.0137 | 0.0118 | 1.1588 | 0.8630 |
| Stomach-esophagus | 0.0097 | 0.0153 | 0.6300 | 1.5874 |
| Muscle-skeleton | 0.0206 | 0.0060 | 3.4261 | 0.2919 |
| Kidneys | 0.0327 | 0.0411 | 0.7960 | 1.2563 |
| Pancreas | 0.0114 | 0.0221 | 0.5143 | 1.9446 |
| Penis | 0.0329 | 0.0000 | undef | 0.0000 |
| Prostate | 0.0286 | 0.0234 | 1.2203 | 0.8195 |
| Uterus | 0.0165 | 0.0071 | 2.3208 | 0.4309 |
| Breast hyperplasia | 0.0145 | | | |
| Small intestine | 0.0187 | | | |
| Prostatic hyperplasia | 0.0119 | | | |
| Seminal vesicles | 0.0178 | | | |
| Sensory organs | 0.0000 | | | |
| White blood cells | 0.0122 | | | |
| | FETUS | | | |
| Development | 0.0000 | | | |
| Gastrointestinal | 0.0154 | | | |
| Brain | 0.0313 | | | |
| Hematopoietic | 0.0039 | | | |
| Heart-blood vessels | 0.0368 | | | |
| Lungs | 0.0111 | | | |
| Kidneys | 0.0124 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0228 | | | |
| Endocrine tissue | 0.0245 | | | |
| Fetal | 0.0035 | | | |
| Gastrointestinal | 0.0244 | | | |
| Hematopoietic | 0.0171 | | | |
| Skin-muscle | 0.0000 | | | |
| Testicles | 0.0156 | | | |
| Lungs | 0.0246 | | | |
| Nerves | 0.0090 | | | |
| Prostate | 0.0192 | | | |
| Sensory organs | 0.0000 | | | |

Electronic Northern for SEQ. ID NO.: 25

| | | | | |
|---|---|---|---|---|
| | NORMAL | | | |
| Bladder | 0.0000 | 0.0051 | 0.0000 | undef |
| Breast | 0.0040 | 0.0131 | 0.3058 | 3.2702 |
| Ovary | 0.0000 | 0.0052 | 0.0000 | undef |
| Endocrine tissue | 0.0036 | 0.0027 | 1.3396 | 0.7465 |
| Gastrointestinal | 0.0039 | 0.0048 | 0.8143 | 1.2281 |
| Brain | 0.0102 | 0.0077 | 1.3270 | 0.7536 |
| Hematopoietic | 0.0084 | 0.0000 | undef | 0.0000 |
| Skin | 0.0000 | 0.0000 | undef | undef |
| Hepatic | 0.0000 | 0.0000 | undef | undef |
| Heart | 0.0000 | 0.0137 | 0.0000 | undef |
| Testicles | 0.0000 | 0.0000 | undef | undef |
| Lungs | 0.0012 | 0.0047 | 0.2634 | 3.7971 |
| Stomach-esophagus | 0.0000 | 0.0000 | undef | undef |
| Muscle-skeleton | 0.0034 | 0.0240 | 0.1428 | 7.0051 |
| Kidneys | 0.0059 | 0.0068 | 0.8683 | 1.1517 |
| Pancreas | 0.0000 | 0.0000 | undef | undef |
| Penis | 0.0030 | 0.0267 | 0.1123 | 8.9040 |
| Prostate | 0.0000 | 0.0064 | 0.0000 | undef |
| Uterus | 0.0017 | 0.0071 | 0.2321 | 4.3088 |
| Breast hyperplasia | 0.0036 | | | |
| Small intestine | 0.0062 | | | |
| Prostatic hyperplasia | 0.0059 | | | |
| Seminal vesicles | 0.0000 | | | |
| Sensory organs | 0.0118 | | | |
| White blood cells | 0.0026 | | | |
| | FETUS | | | |
| Development | 0.0000 | | | |
| Gastrointestinal | 0.0031 | | | |
| Brain | 0.0000 | | | |
| Hematopoietic | 0.0000 | | | |
| Heart-blood vessels | 0.0000 | | | |
| Lungs | 0.0111 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0000 | | | |
| Endocrine tissue | 0.0000 | | | |
| Fetal | 0.0000 | | | |
| Gastrointestinal | 0.0000 | | | |
| Hematopoietic | 0.0000 | | | |
| Skin-muscle | 0.0000 | | | |
| Testicles | 0.0000 | | | |
| Lungs | 0.0000 | | | |
| Nerves | 0.0040 | | | |
| Prostate | 0.0000 | | | |
| Sensory organs | 0.0000 | | | |

Electronic Northern for SEQ. ID NO.: 27

| | | | | |
|---|---|---|---|---|
| | NORMAL | | | |
| Bladder | 0.0093 | 0.0077 | 1.2123 | 0.8249 |
| Breast | 0.0067 | 0.0131 | 0.5096 | 1.9621 |
| Ovary | 0.0000 | 0.0000 | undef | undef |
| Endocrine tissue | 0.0018 | 0.0000 | undef | 0.0000 |
| Gastrointestinal | 0.0019 | 0.0000 | undef | 0.0000 |
| Brain | 0.0042 | 0.0175 | 0.2419 | 4.1338 |
| Hematopoietic | 0.0028 | 0.0000 | undef | 0.0000 |
| Skin | 0.0050 | 0.0000 | undef | 0.0000 |
| Hepatic | 0.0000 | 0.0000 | undef | undef |
| Heart | 0.0064 | 0.0412 | 0.1541 | 6.4872 |
| Testicles | 0.0061 | 0.0000 | undef | 0.0000 |
| Lungs | 0.0062 | 0.0000 | undef | 0.0000 |
| Stomach-esophagus | 0.0000 | 0.0153 | 0.0000 | undef |
| Muscle-skeleton | 0.0017 | 0.0120 | 0.1428 | 7.0051 |
| Kidneys | 0.0089 | 0.0000 | undef | 0.0000 |
| Pancreas | 0.0038 | 0.0000 | undef | 0.0000 |
| Penis | 0.0000 | 0.0267 | 0.0000 | undef |
| Prostate | 0.0095 | 0.0021 | 4.4745 | 0.2235 |
| Uterus | 0.0033 | 0.0142 | 0.2321 | 4.3088 |
| Breast hyperplasia | 0.0036 | | | |
| Small intestine | 0.0000 | | | |
| Prostatic hyperplasia | 0.0030 | | | |
| Seminal vesicles | 0.0000 | | | |
| Sensory organs | 0.0000 | | | |
| White blood cells | 0.0009 | | | |
| | FETUS | | | |
| Development | 0.0307 | | | |
| Gastrointestinal | 0.0062 | | | |
| Brain | 0.0250 | | | |
| Hematopoietic | 0.0197 | | | |
| Heart-blood vessels | 0.0000 | | | |
| Lungs | 0.0185 | | | |
| Kidneys | 0.0062 | | | |
| Prostate | 0.0249 | | | |
| Sensory organs | 0.0558 | | | |
| | STANDARDIZED/ | | | |
| | SUBTRACTED | | | |
| | LIBRARIES | | | |
| Breast | 0.0068 | | | |
| Ovary-uterus | 0.0091 | | | |
| Endocrine tissue | 0.0000 | | | |
| Fetal | 0.0012 | | | |
| Gastrointestinal | 0.0244 | | | |
| Hematopoietic | 0.0000 | | | |
| Skin-muscle | 0.0000 | | | |
| Testicles | 0.0000 | | | |
| Lungs | 0.0082 | | | |
| Nerves | 0.0070 | | | |
| Prostate | 0.0064 | | | |
| Sensory organs | 0.0000 | | | |

Electronic Northern for SEQ. ID NO.: 29

| | | | | |
|---|---|---|---|---|
| | NORMAL | | | |
| Bladder | 0.0000 | 0.0102 | 0.0000 | undef |
| Breast | 0.0013 | 0.0109 | 0.1223 | 8.1755 |
| Ovary | 0.0030 | 0.0052 | 0.5843 | 1.7114 |
| Endocrine tissue | 0.0036 | 0.0082 | 0.4465 | 2.2395 |
| Gastrointestinal | 0.0058 | 0.0000 | undef | 0.0000 |
| Brain | 0.0025 | 0.0307 | 0.0829 | 12.0569 |
| Hematopoietic | 0.0140 | 0.0000 | undef | 0.0000 |
| Skin | 0.0050 | 0.0000 | undef | 0.0000 |
| Hepatic | 0.0050 | 0.0000 | undef | 0.0000 |
| Heart | 0.0000 | 0.0000 | undef | undef |
| Testicles | 0.0000 | 0.0234 | 0.0000 | undef |
| Lungs | 0.0037 | 0.0000 | undef | 0.0000 |
| Stomach-esophagus | 0.0000 | 0.0000 | undef | undef |
| Muscle-skeleton | 0.0051 | 0.0060 | 0.8565 | 1.1675 |
| Kidneys | 0.0000 | 0.0205 | 0.0000 | undef |
| Pancreas | 0.0038 | 0.0000 | undef | 0.0000 |
| Penis | 0.0000 | 0.0000 | undef | undef |
| Prostate | 0.0071 | 0.0043 | 1.6779 | 0.5960 |
| Uterus | 0.0033 | 0.0000 | undef | 0.0000 |
| Breast hyperplasia | 0.0000 | | | |
| Small intestine | 0.0062 | | | |
| Prostatic hyperplasia | 0.0059 | | | |
| Seminal vesicles | 0.0089 | | | |
| Sensory organs | 0.0000 | | | |
| White blood cells | 0.0226 | | | |
| | FETUS | | | |
| Development | 0.0000 | | | |
| Gastrointestinal | 0.0031 | | | |
| Brain | 0.0000 | | | |
| Hematopoietic | 0.0197 | | | |
| Heart-blood vessels | 0.0000 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0204 | | | |
| Ovary-uterus | 0.0023 | | | |
| Endocrine tissue | 0.0000 | | | |
| Fetal | 0.0023 | | | |
| Gastrointestinal | 0.0000 | | | |
| Hematopoietic | 0.0114 | | | |
| Skin-muscle | 0.0000 | | | |
| Testicles | 0.0000 | | | |
| Lungs | 0.0164 | | | |
| Nerves | 0.0030 | | | |
| Prostate | 0.0128 | | | |
| Sensory organs | 0.0000 | | | |

Electronic Northern for SEQ. ID NO.: 30

| | | | | |
|---|---|---|---|---|
| | NORMAL | | | |
| Bladder | 0.0186 | 0.0153 | 1.2123 | 0.8249 |
| Breast | 0.0133 | 0.0436 | 0.3058 | 3.2702 |
| Ovary | 0.0182 | 0.0130 | 1.4023 | 0.7131 |
| Endocrine tissue | 0.0073 | 0.0191 | 0.3827 | 2.6128 |
| Gastrointestinal | 0.0194 | 0.0095 | 2.0357 | 0.4912 |
| Brain | 0.0237 | 0.0449 | 0.5287 | 1.8916 |
| Hematopoietic | 0.0098 | 0.0000 | undef | 0.0000 |
| Skin | 0.0099 | 0.0000 | undef | 0.0000 |
| Hepatic | 0.0050 | 0.0065 | 0.7651 | 1.3069 |
| Heart | 0.0254 | 0.0550 | 0.4624 | 2.1624 |
| Testicles | 0.0244 | 0.0000 | undef | 0.0000 |
| Lungs | 0.0224 | 0.0165 | 1.3544 | 0.7383 |
| Stomach-esophagus | 0.0000 | 0.0307 | 0.0000 | undef |
| Muscle-skeleton | 0.0086 | 0.0180 | 0.4758 | 2.1015 |
| Kidneys | 0.0208 | 0.0205 | 1.0130 | 0.9871 |
| Pancreas | 0.0114 | 0.0387 | 0.2939 | 3.4030 |
| Penis | 0.0120 | 0.0267 | 0.4492 | 2.2260 |
| Prostate | 0.0214 | 0.0128 | 1.6779 | 0.5960 |
| Uterus | 0.0066 | 0.0142 | 0.4642 | 2.1544 |
| Breast hyperplasia | 0.0073 | | | |
| Small intestine | 0.0031 | | | |
| Prostatic hyperplasia | 0.0059 | | | |
| Seminal vesicles | 0.0000 | | | |
| Sensory organs | 0.0118 | | | |
| White blood cells | 0.0305 | | | |
| | FETUS | | | |
| Development | 0.1537 | | | |
| Gastrointestinal | 0.0401 | | | |
| Brain | 0.1126 | | | |
| Hematopoietic | 0.0472 | | | |
| Heart-blood vessels | 0.0164 | | | |
| Lungs | 0.0481 | | | |
| Kidneys | 0.0247 | | | |
| Prostate | 0.0499 | | | |
| Sensory organs | 0.1954 | | | |
| | STANDARDIZED/ | | | |
| | SUBTRACTED | | | |
| | LIBRARIES | | | |
| Breast | 0.0544 | | | |
| Ovary-uterus | 0.0320 | | | |
| Endocrine tissue | 0.0000 | | | |
| Fetal | 0.0636 | | | |
| Gastrointestinal | 0.0610 | | | |
| Hematopoietic | 0.0057 | | | |
| Skin-muscle | 0.1328 | | | |
| Testicles | 0.0000 | | | |
| Lungs | 0.0082 | | | |
| Nerves | 0.0191 | | | |
| Prostate | 0.0064 | | | |
| Sensory organs | 0.0000 | | | |

Electronic Northern for SEQ. ID NO.: 31

| | | | | |
|---|---|---|---|---|
| | NORMAL | | | |
| Bladder | 0.0046 | 0.0026 | 1.8185 | 0.5499 |
| Breast | 0.0027 | 0.0109 | 0.2446 | 4.0878 |
| Ovary | 0.0000 | 0.0078 | 0.0000 | undef |
| Endocrine tissue | 0.0018 | 0.0054 | 0.3349 | 2.9861 |
| Gastrointestinal | 0.0078 | 0.0048 | 1.6285 | 0.6141 |
| Brain | 0.0263 | 0.0110 | 2.3997 | 0.4167 |
| Hematopoietic | 0.0042 | 0.0000 | undef | 0.0000 |
| Skin | 0.0050 | 0.0000 | undef | 0.0000 |
| Hepatic | 0.0099 | 0.0000 | undef | 0.0000 |
| Heart | 0.0064 | 0.0275 | 0.2312 | 4.3248 |
| Testicles | 0.0000 | 0.0117 | 0.0000 | undef |
| Lungs | 0.0112 | 0.0047 | 2.3702 | 0.4219 |
| Stomach-esophagus | 0.0000 | 0.0000 | undef | undef |
| Muscle-skeleton | 0.0051 | 0.0060 | 0.8565 | 1.1675 |
| Kidneys | 0.0089 | 0.0000 | undef | 0.0000 |
| Pancreas | 0.0057 | 0.0000 | undef | 0.0000 |
| Penis | 0.0150 | 0.0000 | undef | 0.0000 |
| Prostate | 0.0048 | 0.0064 | 0.7458 | 1.3409 |
| Uterus | 0.0000 | 0.0000 | undef | undef |
| Breast hyperplasia | 0.0036 | | | |
| Small intestine | 0.0125 | | | |
| Prostatic hyperplasia | 0.0059 | | | |
| Seminal vesicles | 0.0089 | | | |
| Sensory organs | 0.0000 | | | |
| White blood cells | 0.0044 | | | |
| | FETUS | | | |
| Development | 0.0000 | | | |
| Gastrointestinal | 0.0031 | | | |
| Brain | 0.0063 | | | |
| Hematopoietic | 0.0079 | | | |
| Heart-blood vessels | 0.0000 | | | |
| Lungs | 0.0000 | | | |
| Kidneys | 0.0000 | | | |
| Prostate | 0.0249 | | | |
| Sensory organs | 0.0000 | | | |
| | STANDARDIZED/ | | | |
| | SUBTRACTED | | | |
| | LIBRARIES | | | |
| Breast | 0.0136 | | | |
| Ovary-uterus | 0.0091 | | | |
| Endocrine tissue | 0.0000 | | | |
| Fetal | 0.0047 | | | |
| Gastrointestinal | 0.0000 | | | |
| Hematopoietic | 0.0000 | | | |
| Skin-muscle | 0.0227 | | | |
| Testicles | 0.0000 | | | |
| Lungs | 0.0246 | | | |
| Nerves | 0.0231 | | | |
| Prostate | 0.0064 | | | |
| Sensory organs | 0.0000 | | | |

Electronic Northern for SEQ. ID NO.: 33

| | | | | |
|---|---|---|---|---|
| | NORMAL | | | |
| Bladder | 0.0139 | 0.0230 | 0.6062 | 1.6497 |
| Breast | 0.0013 | 0.0131 | 0.1019 | 9.8107 |
| Ovary | 0.0061 | 0.0078 | 0.7791 | 1.2836 |
| Endocrine tissue | 0.0128 | 0.0027 | 4.6885 | 0.2133 |
| Gastrointestinal | 0.0174 | 0.0048 | 3.6642 | 0.2729 |
| Brain | 0.0085 | 0.0142 | 0.5955 | 1.6794 |
| Hematopoietic | 0.0056 | 0.0000 | undef | 0.0000 |
| Skin | 0.0249 | 0.0000 | undef | 0.0000 |
| Hepatic | 0.0000 | 0.0259 | 0.0000 | undef |
| Heart | 0.0159 | 0.0137 | 1.1561 | 0.8650 |
| Testicles | 0.0000 | 0.0000 | undef | undef |
| Lungs | 0.0224 | 0.0260 | 0.8619 | 1.1602 |
| Stomach-esophagus | 0.0097 | 0.0000 | undef | 0.0000 |
| Muscle-skeleton | 0.0154 | 0.0060 | 2.5696 | 0.3892 |
| Kidneys | 0.0149 | 0.0137 | 1.0854 | 0.9213 |
| Pancreas | 0.0038 | 0.0110 | 0.3428 | 2.9168 |
| Penis | 0.0120 | 0.0267 | 0.4492 | 2.2260 |
| Prostate | 0.0191 | 0.0255 | 0.7458 | 1.3409 |
| Uterus | 0.0132 | 0.0071 | 1.8567 | 0.5386 |
| Breast hyperplasia | 0.0145 | | | |
| Small intestine | 0.0093 | | | |
| Prostatic hyperplasia | 0.0208 | | | |
| Seminal vesicles | 0.0000 | | | |
| Sensory organs | 0.0000 | | | |
| White blood cells | 0.0183 | | | |
| | FETUS | | | |
| Development | 0.0000 | | | |
| Gastrointestinal | 0.0123 | | | |
| Brain | 0.0063 | | | |
| Hematopoietic | 0.0000 | | | |
| Heart-blood vessels | 0.0041 | | | |
| Lungs | 0.0037 | | | |
| Kidneys | 0.0124 | | | |
| Prostate | 0.0748 | | | |
| Sensory organs | 0.0000 | | | |
| | STANDARDIZED/ | | | |
| | SUBTRACTED | | | |
| | LIBRARIES | | | |
| Breast | 0.0204 | | | |
| Ovary-uterus | 0.0091 | | | |
| Endocrine tissue | 0.0000 | | | |
| Fetal | 0.0070 | | | |
| Gastrointestinal | 0.0366 | | | |
| Hematopoietic | 0.0114 | | | |
| Skin-muscle | 0.0356 | | | |
| Testicles | 0.0000 | | | |
| Lungs | 0.0164 | | | |
| Nerves | 0.0010 | | | |
| Prostate | 0.0128 | | | |
| Sensory organs | 0.0155 | | | |

Electronic Northern for SEQ. ID NO.: 35

| | | | | |
|---|---|---|---|---|
| | NORMAL | | | |
| Bladder | 0.0139 | 0.0000 | undef | 0.0000 |
| Breast | 0.0013 | 0.0109 | 0.1223 | 8.1755 |
| Ovary | 0.0091 | 0.0078 | 1.1686 | 0.8557 |
| Endocrine tissue | 0.0128 | 0.0082 | 1.5628 | 0.6399 |
| Gastrointestinal | 0.0097 | 0.0095 | 1.0178 | 0.9825 |
| Brain | 0.0068 | 0.0066 | 1.0321 | 0.9689 |
| Hematopoietic | 0.0028 | 0.0000 | undef | 0.0000 |
| Skin | 0.0099 | 0.0000 | undef | 0.0000 |
| Hepatic | 0.0248 | 0.0000 | undef | 0.0000 |
| Heart | 0.0053 | 0.0000 | undef | 0.0000 |
| Testicles | 0.0000 | 0.0000 | undef | undef |
| Lungs | 0.0037 | 0.0095 | 0.3950 | 2.5314 |
| Stomach-esophagus | 0.0000 | 0.0153 | 0.0000 | undef |
| Muscle-skeleton | 0.0069 | 0.0060 | 1.1420 | 0.8756 |
| Kidneys | 0.0149 | 0.0068 | 2.1708 | 0.4607 |
| Pancreas | 0.0057 | 0.0055 | 1.0285 | 0.9723 |
| Penis | 0.0120 | 0.0000 | undef | 0.0000 |
| Prostate | 0.0048 | 0.0064 | 0.7458 | 1.3409 |
| Uterus | 0.0017 | 0.0071 | 0.2321 | 4.3088 |
| Breast hyperplasia | 0.0000 | | | |
| Small intestine | 0.0093 | | | |
| Prostatic hyperplasia | 0.0059 | | | |
| Seminal vesicles | 0.0089 | | | |
| Sensory organs | 0.0118 | | | |
| White blood cells | 0.0017 | | | |
| | FETUS | | | |
| Development | 0.0154 | | | |
| Gastrointestinal | 0.0062 | | | |
| Brain | 0.0063 | | | |
| Hematopoietic | 0.0000 | | | |
| Heart-blood vessels | 0.0041 | | | |
| Lungs | 0.0074 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0068 | | | |
| Ovary-uterus | 0.0091 | | | |
| Endocrine tissue | 0.0000 | | | |
| Fetal | 0.0111 | | | |
| Gastrointestinal | 0.0122 | | | |
| Hematopoietic | 0.0057 | | | |
| Skin-muscle | 0.0453 | | | |
| Testicles | 0.0078 | | | |
| Lungs | 0.0000 | | | |
| Nerves | 0.0100 | | | |
| Prostate | 0.0128 | | | |
| Sensory organs | 0.0000 | | | |

Electronic Northern for SEQ. ID NO.: 36

| | | | | |
|---|---|---|---|---|
| | NORMAL | | | |
| Bladder | 0.0000 | 0.0026 | 0.0000 | undef |
| Breast | 0.0013 | 0.0109 | 0.1223 | 8.1755 |
| Ovary | 0.0000 | 0.0000 | undef | undef |
| Endocrine tissue | 0.0000 | 0.0027 | 0.0000 | undef |
| Gastrointestinal | 0.0019 | 0.0000 | undef | 0.0000 |
| Brain | 0.0051 | 0.0011 | 4.6446 | 0.2153 |
| Hematopoietic | 0.0042 | 0.0000 | undef | 0.0000 |
| Skin | 0.0000 | 0.0000 | undef | undef |
| Hepatic | 0.0000 | 0.0065 | 0.0000 | undef |
| Heart | 0.0000 | 0.0000 | undef | undef |
| Testicles | 0.0000 | 0.0000 | undef | undef |
| Lungs | 0.0000 | 0.0000 | undef | undef |
| Stomach-esophagus | 0.0000 | 0.0000 | undef | undef |
| Muscle-skeleton | 0.0034 | 0.0000 | undef | 0.0000 |
| Kidneys | 0.0000 | 0.0000 | undef | undef |
| Pancreas | 0.0000 | 0.0000 | undef | undef |
| Penis | 0.0000 | 0.0000 | undef | undef |
| Prostate | 0.0048 | 0.0021 | 2.2373 | 0.4470 |
| Uterus | 0.0000 | 0.0000 | undef | undef |
| Breast hyperplasia | 0.0000 | | | |
| Small intestine | 0.0000 | | | |
| Prostatic hyperplasia | 0.0059 | | | |
| Seminal vesicles | 0.0000 | | | |
| Sensory organs | 0.0000 | | | |
| White blood cells | 0.0026 | | | |
| | FETUS | | | |
| Development | 0.0000 | | | |
| Gastrointestinal | 0.0031 | | | |
| Brain | 0.0125 | | | |
| Hematopoietic | 0.0000 | | | |
| Heart-blood vessels | 0.0000 | | | |
| Lungs | 0.0000 | | | |
| Kidneys | 0.0062 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0114 | | | |
| Endocrine tissue | 0.0000 | | | |
| Fetal | 0.0017 | | | |
| Gastrointestinal | 0.0122 | | | |
| Hematopoietic | 0.0000 | | | |
| Skin-muscle | 0.0000 | | | |
| Testicles | 0.0000 | | | |
| Lungs | 0.0000 | | | |
| Nerves | 0.0020 | | | |
| Prostate | 0.0064 | | | |
| Sensory organs | 0.0000 | | | |

Electronic Northern for SEQ. ID NO.: 37

| | | | | |
|---|---|---|---|---|
| | NORMAL | | | |
| Bladder | 0.0046 | 0.0102 | 0.4546 | 2.1996 |
| Breast | 0.0000 | 0.0218 | 0.0000 | undef |
| Ovary | 0.0122 | 0.0442 | 0.2750 | 3.6368 |
| Endocrine tissue | 0.0000 | 0.0027 | 0.0000 | undef |
| Gastrointestinal | 0.0136 | 0.0190 | 0.7125 | 1.4035 |
| Brain | 0.0008 | 0.0077 | 0.1106 | 9.0427 |
| Hematopoietic | 0.0322 | 0.0000 | undef | 0.0000 |
| Skin | 0.0050 | 0.0000 | undef | 0.0000 |
| Hepatic | 0.0000 | 0.0129 | 0.0000 | undef |
| Heart | 0.0021 | 0.0000 | undef | 0.0000 |
| Testicles | 0.0000 | 0.0234 | 0.0000 | undef |
| Lungs | 0.0535 | 0.0473 | 1.1324 | 0.8831 |
| Stomach-esophagus | 0.0676 | 0.0767 | 0.8819 | 1.1339 |
| Muscle-skeleton | 0.0086 | 0.0000 | undef | 0.0000 |
| Kidneys | 0.0000 | 0.0068 | 0.0000 | undef |
| Pancreas | 0.0114 | 0.0055 | 2.0570 | 0.4861 |
| Penis | 0.0030 | 0.0000 | undef | 0.0000 |
| Prostate | 0.0071 | 0.0128 | 0.5593 | 1.7879 |
| Uterus | 0.0017 | 0.0000 | undef | 0.0000 |
| Breast hyperplasia | 0.0000 | | | |
| Small intestine | 0.0249 | | | |
| Prostatic hyperplasia | 0.0030 | | | |
| Seminal vesicles | 0.0000 | | | |
| Sensory organs | 0.0235 | | | |
| White blood cells | 0.1140 | | | |
| | FETUS | | | |
| Development | 0.0154 | | | |
| Gastrointestinal | 0.0154 | | | |
| Brain | 0.0000 | | | |
| Hematopoietic | 0.0039 | | | |
| Heart-blood vessels | 0.0041 | | | |
| Lungs | 0.0037 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0000 | | | |
| Endocrine tissue | 0.0000 | | | |
| Fetal | 0.0029 | | | |
| Gastrointestinal | 0.0366 | | | |
| Hematopoietic | 0.0000 | | | |
| Skin-muscle | 0.0000 | | | |
| Testicles | 0.0000 | | | |
| Lungs | 0.0082 | | | |
| Nerves | 0.0000 | | | |
| Prostate | 0.0000 | | | |
| Sensory organs | 0.0000 | | | |

Electronic Northern for SEQ. ID NO.: 38

| | | | | |
|---|---|---|---|---|
| | NORMAL | | | |
| Bladder | 0.0000 | 0.0026 | 0.0000 | undef |
| Breast | 0.0067 | 0.0131 | 0.5096 | 1.9621 |
| Ovary | 0.0061 | 0.0000 | undef | 0.0000 |
| Endocrine tissue | 0.0036 | 0.0000 | undef | 0.0000 |
| Gastrointestinal | 0.0019 | 0.0000 | undef | 0.0000 |
| Brain | 0.0000 | 0.0033 | 0.0000 | undef |
| Hematopoietic | 0.0084 | 0.0000 | undef | 0.0000 |
| Skin | 0.0000 | 0.0000 | undef | undef |
| Hepatic | 0.0000 | 0.0000 | undef | undef |
| Heart | 0.0021 | 0.0000 | undef | 0.0000 |
| Testicles | 0.0000 | 0.0000 | undef | undef |
| Lungs | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidneys | 0.0119 | 0.0000 | undef | 0.0000 |
| Pancreas | 0.0000 | 0.0000 | undef | undef |
| Penis | 0.0000 | 0.0000 | undef | undef |
| Prostate | 0.0024 | 0.0043 | 0.5593 | 1.7879 |
| Uterus | 0.0017 | 0.0000 | undef | 0.0000 |
| Breast hyperplasia | 0.0073 | | | |
| Small intestine | 0.0031 | | | |
| Prostatic hyperplasia | 0.0030 | | | |
| Seminal vesicles | 0.0000 | | | |
| Sensory organs | 0.0000 | | | |
| White blood cells | 0.0026 | | | |
| | FETUS | | | |
| Development | 0.0000 | | | |
| Gastrointestinal | 0.0000 | | | |
| Brain | 0.0000 | | | |
| Hematopoietic | 0.0000 | | | |
| Heart-blood vessels | 0.0000 | | | |
| Lungs | 0.0000 | | | |
| Kidneys | 0.0124 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0046 | | | |
| Endocrine tissue | 0.0490 | | | |
| Fetal | 0.0029 | | | |
| Gastrointestinal | 0.0000 | | | |
| Hematopoietic | 0.0000 | | | |
| Skin-muscle | 0.0000 | | | |
| Testicles | 0.0000 | | | |
| Lungs | 0.0082 | | | |
| Nerves | 0.0131 | | | |
| Prostate | 0.0000 | | | |
| Sensory organs | 0.0000 | | | |

Electronic Northern for SEQ. ID NO.: 39

| | | | | |
|---|---|---|---|---|
| | NORMAL | | | |
| Bladder | 0.0093 | 0.0077 | 1.2123 | 0.8249 |
| Breast | 0.0053 | 0.0283 | 0.1882 | 5.3141 |
| Ovary | 0.0122 | 0.0130 | 0.9349 | 1.0696 |
| Endocrine tissue | 0.0347 | 0.0300 | 1.1569 | 0.8644 |
| Gastrointestinal | 0.0136 | 0.0000 | undef | 0.0000 |
| Brain | 0.0153 | 0.0131 | 1.1612 | 0.8612 |
| Hematopoietic | 0.0056 | 0.0000 | undef | 0.0000 |
| Skin | 0.0199 | 0.0000 | undef | 0.0000 |
| Hepatic | 0.0198 | 0.0065 | 3.0606 | 0.3267 |
| Heart | 0.0127 | 0.0137 | 0.9249 | 1.0812 |
| Testicles | 0.0122 | 0.0117 | 1.0447 | 0.9572 |
| Lungs | 0.0212 | 0.0165 | 1.2792 | 0.7818 |
| Stomach-esophagus | 0.0193 | 0.0000 | undef | 0.0000 |
| Muscle-skeleton | 0.0120 | 0.0180 | 0.6662 | 1.5011 |
| Kidneys | 0.0030 | 0.0000 | undef | 0.0000 |
| Pancreas | 0.0133 | 0.0000 | undef | 0.0000 |
| Penis | 0.0030 | 0.0533 | 0.0562 | 17.8081 |
| Prostate | 0.0167 | 0.0064 | 2.6101 | 0.3831 |
| Uterus | 0.0066 | 0.0071 | 0.9283 | 1.0772 |
| Breast hyperplasia | 0.0073 | | | |
| Small intestine | 0.0125 | | | |
| Prostatic hyperplasia | 0.0208 | | | |
| Seminal vesicles | 0.0000 | | | |
| Sensory organs | 0.0353 | | | |
| White blood cells | 0.0235 | | | |
| | FETUS | | | |
| Development | 0.0000 | | | |
| Gastrointestinal | 0.0031 | | | |
| Brain | 0.0000 | | | |
| Hematopoietic | 0.0079 | | | |
| Heart-blood vessels | 0.0041 | | | |
| Lungs | 0.0111 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0068 | | | |
| Ovary-uterus | 0.0274 | | | |
| Endocrine tissue | 0.0000 | | | |
| Fetal | 0.0204 | | | |
| Gastrointestinal | 0.0244 | | | |
| Hematopoietic | 0.0000 | | | |
| Skin-muscle | 0.0421 | | | |
| Testicles | 0.0000 | | | |
| Lungs | 0.0164 | | | |
| Nerves | 0.0151 | | | |
| Prostate | 0.0256 | | | |
| Sensory organs | 0.0542 | | | |

Electronic Northern for SEQ. ID NO.: 40

| | | | | |
|---|---|---|---|---|
| | NORMAL | | | |
| Bladder | 0.0000 | 0.0000 | undef | undef |
| Breast | 0.0027 | 0.0174 | 0.1529 | 6.5404 |
| Ovary | 0.0000 | 0.0000 | undef | undef |
| Endocrine tissue | 0.0000 | 0.0082 | 0.0000 | undef |
| Gastrointestinal | 0.0000 | 0.0048 | 0.0000 | undef |
| Brain | 0.0076 | 0.0000 | undef | 0.0000 |
| Hematopoietic | 0.0028 | 0.0000 | undef | 0.0000 |
| Skin | 0.0149 | 0.0000 | undef | 0.0000 |
| Hepatic | 0.0050 | 0.0000 | undef | 0.0000 |
| Heart | 0.0021 | 0.0000 | undef | 0.0000 |
| Testicles | 0.0061 | 0.0000 | undef | 0.0000 |
| Lungs | 0.0087 | 0.0000 | undef | 0.0000 |
| Stomach-esophagus | 0.0097 | 0.0000 | undef | 0.0000 |
| Muscle-skeleton | 0.0051 | 0.0000 | undef | 0.0000 |
| Kidneys | 0.0000 | 0.0000 | undef | undef |
| Pancreas | 0.0000 | 0.0000 | undef | undef |
| Penis | 0.0060 | 0.0000 | undef | 0.0000 |
| Prostate | 0.0024 | 0.0021 | 1.1186 | 0.8939 |
| Uterus | 0.0017 | 0.0000 | undef | 0.0000 |
| Breast hyperplasia | 0.0000 | | | |
| Small intestine | 0.0031 | | | |
| Prostatic hyperplasia | 0.0030 | | | |
| Seminal vesicles | 0.0178 | | | |
| Sensory organs | 0.0000 | | | |
| White blood cells | 0.0070 | | | |
| | FETUS | | | |
| Development | 0.0000 | | | |
| Gastrointestinal | 0.0000 | | | |
| Brain | 0.0000 | | | |
| Hematopoietic | 0.0000 | | | |
| Heart-blood vessels | 0.0000 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0000 | | | |
| Endocrine tissue | 0.0245 | | | |
| Fetal | 0.0117 | | | |
| Gastrointestinal | 0.0000 | | | |
| Hematopoietic | 0.0000 | | | |
| Skin-muscle | 0.0000 | | | |
| Testicles | 0.0000 | | | |
| Lungs | 0.0082 | | | |
| Nerves | 0.0070 | | | |
| Prostate | 0.0000 | | | |
| Sensory organs | 0.0000 | | | |

Electronic Northern for SEQ. ID NO.: 41

| | | | | |
|---|---|---|---|---|
| | NORMAL | | | |
| Bladder | 0.0000 | 0.0128 | 0.0000 | undef |
| Breast | 0.0040 | 0.0218 | 0.1835 | 5.4504 |
| Ovary | 0.0000 | 0.0026 | 0.0000 | undef |
| Endocrine tissue | 0.0018 | 0.0000 | undef | 0.0000 |
| Gastrointestinal | 0.0058 | 0.0095 | 0.6107 | 1.6375 |
| Brain | 0.0000 | 0.0033 | 0.0000 | undef |
| Hematopoietic | 0.0056 | 0.0000 | undef | 0.0000 |
| Skin | 0.0000 | 0.0000 | undef | undef |
| Hepatic | 0.0000 | 0.0000 | undef | undef |
| Heart | 0.0032 | 0.0000 | undef | 0.0000 |
| Testicles | 0.0000 | 0.0000 | undef | undef |
| Lungs | 0.0012 | 0.0047 | 0.2634 | 3.7971 |
| Stomach-esophagus | 0.0000 | 0.0000 | undef | undef |
| Muscle-skeleton | 0.0034 | 0.0000 | undef | 0.0000 |
| Kidneys | 0.0000 | 0.0000 | undef | undef |
| Pancreas | 0.0000 | 0.0055 | 0.0000 | undef |
| Penis | 0.0030 | 0.0000 | undef | 0.0000 |
| Prostate | 0.0048 | 0.0021 | 2.2373 | 0.4470 |
| Uterus | 0.0050 | 0.0000 | undef | 0.0000 |
| Breast hyperplasia | 0.0000 | | | |
| Small intestine | 0.0031 | | | |
| Prostatic hyperplasia | 0.0000 | | | |
| Seminal vesicles | 0.0000 | | | |
| Sensory organs | 0.0000 | | | |
| White blood cells | 0.0061 | | | |
| | FETUS | | | |
| Development | 0.0000 | | | |
| Gastrointestinal | 0.0031 | | | |
| Brain | 0.0000 | | | |
| Hematopoietic | 0.0000 | | | |
| Heart-blood vessels | 0.0000 | | | |
| Lungs | 0.0000 | | | |
| Kidneys | 0.0185 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0000 | | | |
| Endocrine tissue | 0.0000 | | | |
| Fetal | 0.0006 | | | |
| Gastrointestinal | 0.0000 | | | |
| Hematopoietic | 0.0228 | | | |
| Skin-muscle | 0.0000 | | | |
| Testicles | 0.0000 | | | |
| Lungs | 0.0000 | | | |
| Nerves | 0.0000 | | | |
| Prostate | 0.0128 | | | |
| Sensory organs | 0.0000 | | | |

Electronic Northern for SEQ. ID NO.: 42

| | | | | |
|---|---|---|---|---|
| | NORMAL | | | |
| Bladder | 0.0000 | 0.0051 | 0.0000 | undef |
| Breast | 0.0053 | 0.0109 | 0.4893 | 2.0439 |
| Ovary | 0.0030 | 0.0052 | 0.5843 | 1.7114 |
| Endocrine tissue | 0.0000 | 0.0027 | 0.0000 | undef |
| Gastrointestinal | 0.0000 | 0.0000 | undef | undef |
| Brain | 0.0000 | 0.0000 | undef | undef |
| Hematopoietic | 0.0000 | 0.0000 | undef | undef |
| Skin | 0.0000 | 0.0000 | undef | undef |
| Hepatic | 0.0050 | 0.0000 | undef | 0.0000 |
| Heart | 0.0000 | 0.0000 | undef | undef |
| Testicles | 0.0000 | 0.0000 | undef | undef |
| Lungs | 0.0050 | 0.0024 | 2.1069 | 0.4746 |
| Stomach-esophagus | 0.0000 | 0.0000 | undef | undef |
| Muscle-skeleton | 0.0000 | 0.0000 | undef | undef |
| Kidneys | 0.0000 | 0.0000 | undef | undef |
| Pancreas | 0.0000 | 0.0000 | undef | unuef |
| Penis | 0.0000 | 0.0000 | undef | undef |
| Prostate | 0.0048 | 0.0000 | undef | 0.0000 |
| Uterus | 0.0000 | 0.0000 | undef | undef |
| Breast hyperplasia | 0.0036 | | | |
| Small intestine | 0.0000 | | | |
| Prostatic hyperplasia | 0.0000 | | | |
| Seminal vesicles | 0.0000 | | | |
| Sensory organs | 0.0235 | | | |
| White blood cells | 0.0000 | | | |
| | FETUS | | | |
| Development | 0.0000 | | | |
| Gastrointestinal | 0.0000 | | | |
| Brain | 0.0000 | | | |
| Hematopoietic | 0.0000 | | | |
| Heart-blood vessels | 0.0000 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0340 | | | |
| Ovary-uterus | 0.0000 | | | |
| Endocrine tissue | 0.0000 | | | |
| Fetal | 0.0146 | | | |
| Gastrointestinal | 0.0000 | | | |
| Hematopoietic | 0.0000 | | | |
| Skin-muscle | 0.0130 | | | |
| Testicles | 0.0000 | | | |
| Lungs | 0.0082 | | | |
| Nerves | 0.0181 | | | |
| Prostate | 0.0000 | | | |
| Sensory organs | 0.0077 | | | |

Electronic Northern for SEQ. ID NO.: 43

| | | | | |
|---|---|---|---|---|
| | NORMAL | | | |
| Bladder | 0.0000 | 0.0026 | 0.0000 | undef |
| Breast | 0.0013 | 0.0109 | 0.1223 | 8.1755 |
| Ovary | 0.0030 | 0.0000 | undef | 0.0000 |
| Endocrine tissue | 0.0018 | 0.0000 | undef | 0.0000 |
| Gastrointestinal | 0.0000 | 0.0000 | undef | undef |
| Brain | 0.0008 | 0.0000 | undef | 0.0000 |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0000 | 0.0024 | 0.0000 | undef |
| Stomach-esophagus | 0.0000 | 0.0000 | undef | undef |
| Muscle-skeleton | 0.0000 | 0.0000 | undef | undef |
| Kidneys | 0.0030 | 0.0000 | undef | 0.0000 |
| Pancreas | 0.0000 | 0.0000 | undef | undef |
| Penis | 0.0000 | 0.0267 | 0.0000 | undef |
| Prostate | 0.0000 | 0.0000 | undef | undef |
| Uterus | 0.0017 | 0.0000 | undef | 0.0000 |
| Breast hyperplasia | 0.0000 | | | |
| Small intestine | 0.0000 | | | |
| Prostatic hyperplasia | 0.0000 | | | |
| Seminal vesicles | 0.0000 | | | |
| Sensory organs | 0.0118 | | | |
| White blood cells | 0.0000 | | | |
| | FETUS | | | |
| Development | 0.0000 | | | |
| Gastrointestinal | 0.0000 | | | |
| Brain | 0.0000 | | | |
| Hematopoietic | 0.0000 | | | |
| Heart-blood vessels | 0.0000 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0000 | | | |
| Endocrine tissue | 0.0000 | | | |
| Fetal | 0.0029 | | | |
| Gastrointestinal | 0.0000 | | | |
| Hematopoietic | 0.0000 | | | |
| Skin-muscle | 0.0000 | | | |
| Testicles | 0.0000 | | | |
| Lungs | 0.0000 | | | |
| Nerves | 0.0010 | | | |
| Prostate | 0.0064 | | | |
| Sensory organs | 0.0000 | | | |

Electronic Northern for SEQ. ID NO.: 44

| | | | | |
|---|---|---|---|---|
| | NORMAL | | | |
| Bladder | 0.0000 | 0.0000 | undef | undef |
| Breast | 0.0013 | 0.0153 | 0.0874 | 11.4458 |
| Ovary | 0.0000 | 0.0052 | 0.0000 | undef |
| Endocrine tissue | 0.0000 | 0.0000 | undef | undef |
| Gastrointestinal | 0.0000 | 0.0048 | 0.0000 | undef |
| Brain | 0.0000 | 0.0000 | undef | unaef |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0012 | 0.0000 | undef | 0.0000 |
| Stomach-esophagus | 0.0193 | 0.0000 | undef | 0.0000 |
| Muscle-skeleton | 0.0000 | 0.0000 | undef | undef |
| Kidneys | 0.0000 | 0.0000 | undef | undef |
| Pancreas | 0.0000 | 0.0000 | undef | undef |
| Penis | 0.0060 | 0.0000 | undef | 0.0000 |
| Prostate | 0.0000 | 0.0000 | undef | undef |
| Uterus | 0.0000 | 0.0000 | undef | undef |
| Breast hyperplasia | 0.0000 | | | |
| Small intestine | 0.0000 | | | |
| Prostatic hyperplasia | 0.0000 | | | |
| Seminal vesicles | 0.0000 | | | |
| Sensory organs | 0.0000 | | | |
| White blood cells | 0.0000 | | | |
| | FETUS | | | |
| Development | 0.0000 | | | |
| Gastrointestinal | 0.0000 | | | |
| Brain | 0.0000 | | | |
| Hematopoietic | 0.0039 | | | |
| Heart-blood vessels | 0.0041 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0023 | | | |
| Endocrine tissue | 0.0000 | | | |
| Fetal | 0.0000 | | | |
| Gastrointestinal | 0.0000 | | | |
| Hematopoietic | 0.0000 | | | |
| Skin-muscle | 0.0000 | | | |
| Testicles | 0.0000 | | | |
| Lungs | 0.0000 | | | |
| Nerves | 0.0010 | | | |
| Prostate | 0.0000 | | | |
| Sensory organs | 0.0000 | | | |

Electronic Northern for SEQ. ID NO.: 45

| | | | | |
|---|---|---|---|---|
| | NORMAL | | | |
| Bladder | 0.0139 | 0.0000 | undef | 0.0000 |
| Breast | 0.0093 | 0.0196 | 0.4757 | 2.1023 |
| Ovary | 0.0122 | 0.0078 | 1.5582 | 0.6418 |
| Endocrine tissue | 0.0036 | 0.0054 | 0.6698 | 1.4930 |
| Gastrointestinal | 0.0097 | 0.0000 | undef | 0.0000 |
| Brain | 0.0110 | 0.0131 | 0.8386 | 1.1924 |
| Hematopoietic | 0.0028 | 0.0378 | 0.0739 | 13.5274 |
| Skin | 0.0348 | 0.0000 | undef | 0.0000 |
| Hepatic | 0.0099 | 0.0000 | undef | 0.0000 |
| Heart | 0.0106 | 0.0000 | undef | 0.0000 |
| Testicles | 0.0061 | 0.0117 | 0.5224 | 1.9144 |
| Lungs | 0.0112 | 0.0095 | 1.1851 | 0.8438 |
| Stomach-esophagus | 0.0000 | 0.0000 | undef | undef |
| Muscle-skeleton | 0.0051 | 0.0060 | 0.8565 | 1.1675 |
| Kidneys | 0.0089 | 0.0068 | 1.3025 | 0.7678 |
| Pancreas | 0.0133 | 0.0055 | 2.3999 | 0.4167 |
| Penis | 0.0090 | 0.0000 | undef | 0.0000 |
| Prostate | 0.0095 | 0.0043 | 2.2373 | 0.4470 |
| Uterus | 0.0066 | 0.0071 | 0.9283 | 1.0772 |
| Breast hperplasia | 0.0109 | | | |
| Small intestine | 0.0062 | | | |
| Prostatic hyperplasia | 0.0030 | | | |
| Seminal vesicles | 0.0089 | | | |
| Sensory organs | 0.0235 | | | |
| White blood cells | 0.0078 | | | |
| | FETUS | | | |
| Development | 0.0154 | | | |
| Gastrointestinal | 0.0092 | | | |
| Brain | 0.0125 | | | |
| Hematopoietic | 0.0000 | | | |
| Heart-blood vessels | 0.0082 | | | |
| Lungs | 0.0296 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0046 | | | |
| Endocrine tissue | 0.0000 | | | |
| Fetal | 0.0210 | | | |
| Gastrointestinal | 0.0122 | | | |
| Hematopoietic | 0.0114 | | | |
| Skin-muscle | 0.0032 | | | |
| Testicles | 0.0000 | | | |
| Lungs | 0.0164 | | | |
| Nerves | 0.0131 | | | |
| Prostate | 0.0000 | | | |
| Sensory organs | 0.0000 | | | |

Electronic Northern for SEQ. ID NO.: 46

| | | | | |
|---|---|---|---|---|
| | NORMAL | | | |
| Bladder | 0.0232 | 0.0051 | 4.5462 | 0.2200 |
| Breast | 0.0027 | 0.0153 | 0.1747 | 5.7229 |
| Ovary | 0.0030 | 0.0078 | 0.3895 | 2.5671 |
| Endocrine tissue | 0.0000 | 0.0054 | 0.0000 | undef |
| Gastrointestinal | 0.0019 | 0.0095 | 0.2036 | 4.9124 |
| Brain | 0.0076 | 0.0099 | 0.7741 | 1.2918 |
| Hematopoietic | 0.0028 | 0.0000 | undef | 0.0000 |
| Skin | 0.0000 | 0.0000 | undef | undef |
| Hepatic | 0.0050 | 0.0065 | 0.7651 | 1.3069 |
| Heart | 0.0053 | 0.0550 | 0.0963 | 10.3795 |
| Testicles | 0.0122 | 0.0117 | 1.0447 | 0.9572 |
| Lungs | 0.0037 | 0.0071 | 0.5267 | 1.8986 |
| Stomach-esophagus | 0.0290 | 0.0077 | 3.7798 | 0.2646 |
| Muscle-skeleton | 0.0017 | 0.0000 | undef | 0.0000 |
| Kidneys | 0.0089 | 0.0068 | 1.3025 | 0.7678 |
| Pancreas | 0.0076 | 0.0055 | 1.3713 | 0.7292 |
| Penis | 0.0030 | 0.0000 | undef | 0.0000 |
| Prostate | 0.0024 | 0.0106 | 0.2237 | 4.4697 |
| Uterus | 0.0083 | 0.0000 | undef | 0.0000 |
| Breast hyperplasia | 0.0000 | | | |
| Small intestine | 0.0062 | | | |
| Prostatic hyperplasia | 0.0089 | | | |
| Seminal vesicles | 0.0000 | | | |
| Sensory organs | 0.0000 | | | |
| White blood cells | 0.0017 | | | |
| | FETUS | | | |
| Development | 0.0000 | | | |
| Gastrointestinal | 0.0000 | | | |
| Brain | 0.0063 | | | |
| Hematopoietic | 0.0000 | | | |
| Heart-blood vessels | 0.0041 | | | |
| Lungs | 0.0074 | | | |
| Kidneys | 0.0247 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0068 | | | |
| Endocrine tissue | 0.0000 | | | |
| Fetal | 0.0017 | | | |
| Gastrointestinal | 0.0122 | | | |
| Hematopoietic | 0.0000 | | | |
| Skin-muscle | 0.0000 | | | |
| Testicles | 0.0000 | | | |
| Lungs | 0.0082 | | | |
| Nerves | 0.0020 | | | |
| Prostate | 0.0128 | | | |
| Sensory organs | 0.0000 | | | |

Electronic Northern for SEQ. ID NO.: 47

| | | | | |
|---|---|---|---|---|
| | NORMAL | | | |
| Bladder | 0.0000 | 0.0051 | 0.0000 | undef |
| Breast | 0.0013 | 0.0131 | 0.1019 | 9.8107 |
| Ovary | 0.0030 | 0.0052 | 0.5843 | 1.7114 |
| Endocrine tissue | 0.0055 | 0.0000 | undef | 0.0000 |
| Gastrointestinal | 0.0000 | 0.0000 | undef | undef |
| Brain | 0.0068 | 0.0055 | 1.2386 | 0.8074 |
| Hematopoietic | 0.0042 | 0.0000 | undef | 0.0000 |
| Skin | 0.0000 | 0.0000 | undef | undef |
| Hepatic | 0.0099 | 0.0000 | undef | 0.0000 |
| Heart | 0.0032 | 0.0000 | undef | 0.0000 |
| Testicles | 0.0000 | 0.0000 | undef | undef |
| Lungs | 0.0000 | 0.0047 | 0.0000 | undef |
| Stomach-esophagus | 0.0000 | 0.0153 | 0.0000 | undef |
| Muscle-skeleton | 0.0034 | 0.0000 | undef | 0.0000 |
| Kidneys | 0.0089 | 0.0068 | 1.3025 | 0.7678 |
| Pancreas | 0.0000 | 0.0000 | undef | undef |
| Penis | 0.0030 | 0.0000 | undef | 0.0000 |
| Prostate | 0.0000 | 0.0085 | 0.0000 | undef |
| Uterus | 0.0033 | 0.0000 | undef | 0.0000 |
| Breast hyperplasia | 0.0073 | | | |
| Small intestine | 0.0062 | | | |
| Prostatic hyperplasia | 0.0059 | | | |
| Seminal vesicles | 0.0178 | | | |
| Sensory organs | 0.0000 | | | |
| White blood cells | 0.0009 | | | |
| | FETUS | | | |
| Development | 0.0154 | | | |
| Gastrointestinal | 0.0031 | | | |
| Brain | 0.0063 | | | |
| Hematopoietic | 0.0000 | | | |
| Heart-blood vessels | 0.0000 | | | |
| Lungs | 0.0000 | | | |
| Kidneys | 0.0062 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0023 | | | |
| Endocrine tissue | 0.0000 | | | |
| Fetal | 0.0023 | | | |
| Gastrointestinal | 0.0000 | | | |
| Hematopoietic | 0.0000 | | | |
| Skin-muscle | 0.0065 | | | |
| Testicles | 0.0000 | | | |
| Lungs | 0.0082 | | | |
| Nerves | 0.0000 | | | |
| Prostate | 0.0000 | | | |
| Sensory organs | 0.0000 | | | |

Electronic Northern for SEQ. ID NO.: 48

| | | | | |
|---|---|---|---|---|
| | NORMAL | | | |
| Bladder | 0.0093 | 0.0051 | 1.8185 | 0.5499 |
| Breast | 0.0053 | 0.0196 | 0.2718 | 3.6790 |
| Ovary | 0.0061 | 0.0052 | 1.1686 | 0.8557 |
| Endocrine tissue | 0.0073 | 0.0109 | 0.6698 | 1.4930 |
| Gastrointestinal | 0.0097 | 0.0143 | 0.6786 | 1.4737 |
| Brain | 0.0059 | 0.0022 | 2.7094 | 0.3691 |
| Hematopoietic | 0.0042 | 0.0378 | 0.1109 | 9.0183 |
| Skin | 0.0050 | 0.0000 | undef | 0.0000 |
| Hepatic | 0.0000 | 0.0065 | 0.0000 | undef |
| Heart | 0.0138 | 0.0000 | undef | 0.0000 |
| Testicles | 0.0122 | 0.0234 | 0.5224 | 1.9144 |
| Lungs | 0.0012 | 0.0071 | 0.1756 | 5.6957 |
| Stomach-esophagus | 0.0290 | 0.0000 | undef | 0.0000 |
| Muscle-skeleton | 0.0051 | 0.0120 | 0.4283 | 2.3350 |
| Kidneys | 0.0000 | 0.0000 | undef | undef |
| Pancreas | 0.0095 | 0.0000 | undef | 0.0000 |
| Penis | 0.0150 | 0.0000 | undef | 0.0000 |
| Prostate | 0.0048 | 0.0085 | 0.5593 | 1.7879 |
| Uterus | 0.0050 | 0.0071 | 0.6963 | 1.4363 |
| Breast hyperplasia | 0.0000 | | | |
| Small intestine | 0.0093 | | | |
| Prostatic hyperplasia | 0.0089 | | | |
| Seminal vesicles | 0.0000 | | | |
| Sensory organs | 0.0000 | | | |
| White blood cells | 0.0044 | | | |
| | FETUS | | | |
| Development | 0.0307 | | | |
| Gastrointestinal | 0.0062 | | | |
| Brain | 0.0063 | | | |
| Hematopoietic | 0.0118 | | | |
| Heart-blood vessels | 0.0164 | | | |
| Lungs | 0.0148 | | | |
| Kidneys | 0.0062 | | | |
| Prostate | 0.0499 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0137 | | | |
| Endocrine tissue | 0.0000 | | | |
| Fetal | 0.0187 | | | |
| Gastrointestinal | 0.0244 | | | |
| Hematopoietic | 0.0000 | | | |
| Skin-muscle | 0.0194 | | | |
| Testicles | 0.0156 | | | |
| Lungs | 0.0000 | | | |
| Nerves | 0.0131 | | | |
| Prostate | 0.0321 | | | |
| Sensory organs | 0.0000 | | | |

Electronic Northern for SEQ. ID NO.: 49

| | | | | |
|---|---|---|---|---|
| | NORMAL | | | |
| Bladder | 0.0000 | 0.0077 | 0.0000 | undef |
| Breast | 0.0040 | 0.0153 | 0.2621 | 3.8153 |
| Ovary | 0.0091 | 0.0286 | 0.3187 | 3.1376 |
| Endocrine tissue | 0.0182 | 0.0327 | 0.5582 | 1.7916 |
| Gastrointestinal | 0.0155 | 0.0095 | 1.6285 | 0.6141 |
| Brain | 0.0127 | 0.0099 | 1.2902 | 0.7751 |
| Hematopoietic | 0.0098 | 0.0000 | undef | 0.0000 |
| Skin | 0.0000 | 0.0000 | undef | undef |
| Hepatic | 0.0099 | 0.0000 | undef | 0.0000 |
| Heart | 0.0265 | 0.0275 | 0.9634 | 1.0380 |
| Testicles | 0.0061 | 0.0117 | 0.5224 | 1.9144 |
| Lungs | 0.0149 | 0.0260 | 0.5746 | 1.7403 |
| Stomach-esophagus | 0.0097 | 0.0077 | 1.2599 | 0.7937 |
| Muscle-skeleton | 0.0103 | 0.0060 | 1.7130 | 0.5838 |
| Kidneys | 0.0208 | 0.0342 | 0.6078 | 1.6452 |
| Pancreas | 0.0019 | 0.0055 | 0.3428 | 2.9168 |
| Penis | 0.0060 | 0.0000 | undef | 0.0000 |
| Prostate | 0.0024 | 0.0106 | 0.2237 | 4.4697 |
| Uterus | 0.0099 | 0.0000 | undef | 0.0000 |
| Breast hyperplasia | 0.0000 | | | |
| Small intestine | 0.0093 | | | |
| Prostatic hyperplasia | 0.0059 | | | |
| Seminal vesicles | 0.0178 | | | |
| Sensory organs | 0.0118 | | | |
| White blood cells | 0.0052 | | | |
| | FETUS | | | |
| Development | 0.0000 | | | |
| Gastrointestinal | 0.0062 | | | |
| Brain | 0.0125 | | | |
| Hematopoietic | 0.0236 | | | |
| Heart-blood vessels | 0.0286 | | | |
| Lungs | 0.0074 | | | |
| Kidneys | 0.0062 | | | |
| Prostate | 0.0000 | | | |
| Sensory organs | 0.0279 | | | |
| | STANDARDIZED/ | | | |
| | SUBTRACTED | | | |
| | LIBRARIES | | | |
| Breast | 0.0000 | | | |
| Ovary-uterus | 0.0114 | | | |
| Endocrine tissue | 0.0000 | | | |
| Fetal | 0.0210 | | | |
| Gastrointestinal | 0.0244 | | | |
| Hematopoietic | 0.0000 | | | |
| Skin-muscle | 0.0421 | | | |
| Testicles | 0.0000 | | | |
| Lungs | 0.0164 | | | |
| Nerves | 0.0050 | | | |
| Prostate | 0.0064 | | | |
| Sensory organs | 0.0155 | | | |

Electronic Northern for SEQ. ID NO.: 50

| | | | | |
|---|---|---|---|---|
| | NORMAL | | | |
| Bladder | 0.0000 | 0.0179 | 0.0000 | undef |
| Breast | 0.0013 | 0.0065 | 0.2039 | 4.9053 |
| Ovary | 0.0000 | 0.0000 | undef | undef |
| Endocrine tissue | 0.0018 | 0.0000 | undef | 0.0000 |
| Gastrointestinal | 0.0019 | 0.0000 | undef | 0.0000 |
| Brain | 0.0008 | 0.0022 | 0.3871 | 2.5836 |
| Hematopoietic | 0.0014 | 0.0000 | undef | 0.0000 |
| Skin | 0.0000 | 0.0000 | undef | undef |
| Hepatic | 0.0050 | 0.0000 | undef | 0.0000 |
| Heart | 0.0032 | 0.0000 | undef | 0.0000 |
| Testicles | 0.0000 | 0.0000 | undef | undef |
| Lungs | 0.0012 | 0.0047 | 0.2634 | 3.7971 |
| Stomach-esophagus | 0.0000 | 0.0000 | undef | undef |
| Muscle-skeleton | 0.0017 | 0.0060 | 0.2855 | 3.5025 |
| Kidneys | 0.0000 | 0.0000 | undef | undef |
| Pancreas | 0.0000 | 0.0055 | 0.0000 | undef |
| Penis | 0.0000 | 0.0000 | undef | undef |
| Prostate | 0.0000 | 0.0000 | undef | undef |
| Uterus | 0.0083 | 0.0000 | undef | 0.0000 |
| Breast hyperplasia | 0.0000 | | | |
| Small intestine | 0.0000 | | | |
| Prostatic hyperplasia | 0.0000 | | | |
| Seminal vesicles | 0.0000 | | | |
| Sensory organs | 0.0000 | | | |
| White blood cells | 0.0009 | | | |
| | FETUS | | | |
| Development | 0.0000 | | | |
| Gastrointestinal | 0.0000 | | | |
| Brain | 0.0000 | | | |
| Hematopoietic | 0.0000 | | | |
| Heart-blood vessels | 0.0000 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0046 | | | |
| Endocrine tissue | 0.0000 | | | |
| Fetal | 0.0064 | | | |
| Gastrointestinal | 0.0000 | | | |
| Hematopoietic | 0.0000 | | | |
| Skin-muscle | 0.0065 | | | |
| Testicles | 0.0000 | | | |
| Lungs | 0.0164 | | | |
| Nerves | 0.0040 | | | |
| Prostate | 0.0000 | | | |
| Sensory organs | 0.0000 | | | |

Electronic Northern for SEQ. ID NO.: 51

| | | | | |
|---|---|---|---|---|
| | NORMAL | | | |
| Bladder | 0.0000 | 0.0051 | 0.0000 | undef |
| Breast | 0.0053 | 0.0153 | 0.3495 | 2.8614 |
| Ovary | 0.0213 | 0.0234 | 0.9089 | 1.1002 |
| Endocrine tissue | 0.0182 | 0.0518 | 0.3525 | 2.8368 |
| Gastrointestinal | 0.0078 | 0.0048 | 1.6285 | 0.6141 |
| Brain | 0.0136 | 0.0120 | 1.1260 | 0.8881 |
| Hematopoietic | 0.0098 | 0.0000 | undef | 0.0000 |
| Skin | 0.0000 | 0.0000 | undef | undef |
| Hepatic | 0.0000 | 0.0194 | 0.0000 | undef |
| Heart | 0.0095 | 0.0000 | undef | 0.0000 |
| Testicles | 0.0428 | 0.0117 | 3.6565 | 0.2735 |
| Lungs | 0.0137 | 0.0142 | 0.9656 | 1.0356 |
| Stomach-esophagus | 0.0097 | 0.0000 | undef | 0.0000 |
| Muscle-skeleton | 0.0017 | 0.0600 | 0.0286 | 35.0255 |
| Kidneys | 0.0178 | 0.0479 | 0.3721 | 2.6872 |
| Pancreas | 0.0170 | 0.0000 | undef | 0.0000 |
| Penis | 0.0150 | 0.0000 | undef | 0.0000 |
| Prostate | 0.0071 | 0.0383 | 0.1864 | 5.3637 |
| Uterus | 0.0066 | 0.0071 | 0.9283 | 1.0772 |
| Breast hyperplasia | 0.0218 | | | |
| Small intestine | 0.0000 | | | |
| Prostatic hyperplasia | 0.0208 | | | |
| Seminal vesicles | 0.0089 | | | |
| Sensory organs | 0.0353 | | | |
| White blood cells | 0.0218 | | | |
| | FETUS | | | |
| Development | 0.0154 | | | |
| Gastrointestinal | 0.0123 | | | |
| Brain | 0.0000 | | | |
| Hematopoietic | 0.0039 | | | |
| Heart-blood vessels | 0.0164 | | | |
| Lungs | 0.0037 | | | |
| Kidneys | 0.0185 | | | |
| Prostate | 0.0249 | | | |
| Sensory organs | 0.0140 | | | |
| | STANDARDIZED/ | | | |
| | SUBTRACTED | | | |
| | LIBRARIES | | | |
| Breast | 0.0000 | | | |
| Ovary-uterus | 0.0434 | | | |
| Endocrine tissue | 0.0000 | | | |
| Fetal | 0.0210 | | | |
| Gastrointestinal | 0.0366 | | | |
| Hematopoietic | 0.0000 | | | |
| Skin-muscle | 0.0972 | | | |
| Testicles | 0.0468 | | | |
| Lungs | 0.0082 | | | |
| Nerves | 0.0151 | | | |
| Prostate | 0.0321 | | | |
| Sensory organs | 0.0310 | | | |

Electronic Northern for SEQ. ID NO.: 52

| | | | | |
|---|---|---|---|---|
| | NORMAL | | | |
| Bladder | 0.0232 | 0.0256 | 0.9092 | 1.0998 |
| Breast | 0.0053 | 0.0131 | 0.4077 | 2.4527 |
| Ovary | 0.0061 | 0.0078 | 0.7791 | 1.2836 |
| Endocrine tissue | 0.0109 | 0.0054 | 2.0093 | 0.4977 |
| Gastrointestinal | 0.0097 | 0.0000 | undef | 0.0000 |
| Brain | 0.0042 | 0.0131 | 0.3225 | 3.1004 |
| Hematopoietic | 0.0098 | 0.0000 | undef | 0.0000 |
| Skin | 0.0249 | 0.0000 | undef | 0.0000 |
| Hepatic | 0.0000 | 0.0000 | undef | undef |
| Heart | 0.0201 | 0.0000 | undef | 0.0000 |
| Testicles | 0.0000 | 0.0117 | 0.0000 | undef |
| Lungs | 0.0125 | 0.0118 | 1.0534 | 0.9493 |
| Stomach-esophagus | 0.0386 | 0.0153 | 2.5198 | 0.3968 |
| Muscle-skeleton | 0.0034 | 0.0060 | 0.5710 | 1.7513 |
| Kidneys | 0.0119 | 0.0137 | 0.8683 | 1.1517 |
| Pancreas | 0.0038 | 0.0110 | 0.3428 | 2.9168 |
| Penis | 0.0090 | 0.0533 | 0.1685 | 5.9360 |
| Prostate | 0.0119 | 0.0170 | 0.6991 | 1.4303 |
| Uterus | 0.0099 | 0.0142 | 0.6963 | 1.4363 |
| Breast hyperplasia | 0.0073 | | | |
| Small intestine | 0.0125 | | | |
| Prostatic hyperplasia | 0.0119 | | | |
| Seminal vesicles | 0.0178 | | | |
| Sensory organs | 0.0000 | | | |
| White blood cells | 0.0052 | | | |
| | FETUS | | | |
| Development | 0.0307 | | | |
| Gastrointestinal | 0.0062 | | | |
| Brain | 0.0063 | | | |
| Hematopoietic | 0.0236 | | | |
| Heart-blood vessels | 0.0041 | | | |
| Lungs | 0.0037 | | | |
| Kidneys | 0.0000 | | | |
| Prostate | 0.0748 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0205 | | | |
| Endocrine tissue | 0.0000 | | | |
| Fetal | 0.0128 | | | |
| Gastrointestinal | 0.0244 | | | |
| Hematopoietic | 0.0057 | | | |
| Skin-muscle | 0.0130 | | | |
| Testicles | 0.0078 | | | |
| Lungs | 0.0000 | | | |
| Nerves | 0.0030 | | | |
| Prostate | 0.0128 | | | |
| Sensory organs | 0.0000 | | | |

Electronic Northern for SEQ. ID NO.: 53

| | | | | |
|---|---|---|---|---|
| | NORMAL | | | |
| Bladder | 0.0093 | 0.0051 | 1.8185 | 0.5499 |
| Breast | 0.0053 | 0.0153 | 0.3495 | 2.8614 |
| Ovary | 0.0000 | 0.0026 | 0.0000 | undef |
| Endocrine tissue | 0.0018 | 0.0000 | undef | 0.0000 |
| Gastrointestinal | 0.0039 | 0.0143 | 0.2714 | 3.6843 |
| Brain | 0.0034 | 0.0164 | 0.2064 | 4.8443 |
| Hematopoietic | 0.0028 | 0.0000 | undef | 0.0000 |
| Skin | 0.0050 | 0.0000 | undef | 0.0000 |
| Hepatic | 0.0000 | 0.0194 | 0.0000 | undef |
| Heart | 0.0032 | 0.0000 | undef | 0.0000 |
| Testicles | 0.0061 | 0.0000 | undef | 0.0000 |
| Lungs | 0.0075 | 0.0047 | 1.5801 | 0.6329 |
| Stomach-esophagus | 0.0000 | 0.0000 | undef | undef |
| Muscle-skeleton | 0.0034 | 0.0180 | 0.1903 | 5.2538 |
| Kidneys | 0.0059 | 0.0137 | 0.4342 | 2.3033 |
| Pancreas | 0.0019 | 0.0055 | 0.3428 | 2.9168 |
| Penis | 0.0060 | 0.0000 | undef | 0.0000 |
| Prostate | 0.0024 | 0.0043 | 0.5593 | 1.7879 |
| Uterus | 0.0066 | 0.0000 | undef | 0.0000 |
| Breast hyperplasia | 0.0073 | | | |
| Small intestine | 0.0093 | | | |
| Prostatic hyperplasia | 0.0030 | | | |
| Seminal vesicles | 0.0000 | | | |
| Sensory organs | 0.0118 | | | |
| White blood cells | 0.0000 | | | |
| | FETUS | | | |
| Development | 0.0000 | | | |
| Gastrointestinal | 0.0031 | | | |
| Brain | 0.0000 | | | |
| Hematopoietic | 0.0079 | | | |
| Heart-blood vessels | 0.0000 | | | |
| Lungs | 0.0000 | | | |
| Kidneys | 0.0062 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0046 | | | |
| Endocrine tissue | 0.0000 | | | |
| Fetal | 0.0006 | | | |
| Gastrointestinal | 0.0000 | | | |
| Hematopoietic | 0.0057 | | | |
| Skin-muscle | 0.0097 | | | |
| Testicles | 0.0000 | | | |
| Lungs | 0.0082 | | | |
| Nerves | 0.0050 | | | |
| Prostate | 0.0000 | | | |
| Sensory organs | 0.0000 | | | |

Electronic Northern for SEQ. ID NO.: 54

| | | | | |
|---|---|---|---|---|
| | NORMAL | | | |
| Bladder | 0.0139 | 0.0102 | 1.3639 | 0.7332 |
| Breast | 0.0080 | 0.0218 | 0.3669 | 2.7252 |
| Ovary | 0.0122 | 0.0182 | 0.6678 | 1.4975 |
| Endocrine tissue | 0.0128 | 0.0191 | 0.6698 | 1.4930 |
| Gastrointestinal | 0.0097 | 0.0286 | 0.3393 | 2.9474 |
| Brain | 0.0059 | 0.0110 | 0.5419 | 1.8454 |
| Hematopoietic | 0.0280 | 0.0000 | undef | 0.0000 |
| Skin | 0.0050 | 0.0000 | undef | 0.0000 |
| Hepatic | 0.0000 | 0.0129 | 0.0000 | undef |
| Heart | 0.0127 | 0.0000 | undef | 0.0000 |
| Testicles | 0.0122 | 0.0117 | 1.0447 | 0.9572 |
| Lungs | 0.0100 | 0.0071 | 1.4046 | 0.7120 |
| Stomach-esophagus | 0.0000 | 0.0153 | 0.0000 | undef |
| Muscle-skeleton | 0.0223 | 0.0000 | undef | 0.0000 |
| Kidneys | 0.0149 | 0.0137 | 1.0854 | 0.9213 |
| Pancreas | 0.0133 | 0.0442 | 0.3000 | 3.3335 |
| Penis | 0.0060 | 0.0000 | undef | 0.0000 |
| Prostate | 0.0095 | 0.0128 | 0.7458 | 1.3409 |
| Uterus | 0.0264 | 0.0000 | undef | 0.0000 |
| Breast hyperplasia | 0.0036 | | | |
| Small intestine | 0.0125 | | | |
| Prostatic hyperplasia | 0.0238 | | | |
| Seminal vesicles | 0.0267 | | | |
| Sensory organs | 0.0118 | | | |
| White blood cells | 0.0026 | | | |
| | FETUS | | | |
| Development | 0.0000 | | | |
| Gastrointestinal | 0.0123 | | | |
| Brain | 0.0125 | | | |
| Hematopoietic | 0.0118 | | | |
| Heart-blood vessels | 0.0000 | | | |
| Lungs | 0.0111 | | | |
| Kidneys | 0.0309 | | | |
| Prostate | 0.0249 | | | |
| Sensory organs | 0.0000 | | | |
| | STANDARDIZED/ | | | |
| | SUBTRACTED | | | |
| | LIBRARIES | | | |
| Breast | 0.0136 | | | |
| Ovary-uterus | 0.0274 | | | |
| Endocrine tissue | 0.0245 | | | |
| Fetal | 0.0099 | | | |
| Gastrointestinal | 0.0122 | | | |
| Hematopoietic | 0.0228 | | | |
| Skin-muscle | 0.0324 | | | |
| Testicles | 0.0000 | | | |
| Lungs | 0.0082 | | | |
| Nerves | 0.0131 | | | |
| Prostate | 0.0000 | | | |
| Sensory organs | 0.0077 | | | |

Electronic Northern for SEQ. ID NO.: 55

| | | | | |
|---|---|---|---|---|
| | NORMAL | | | |
| Bladder | 0.0046 | 0.0051 | 0.9092 | 1.0998 |
| Breast | 0.0067 | 0.0153 | 0.4368 | 2.2892 |
| Ovary | 0.0000 | 0.0130 | 0.0000 | undef |
| Endocrine tissue | 0.0091 | 0.0136 | 0.6698 | 1.4930 |
| Gastrointestinal | 0.0136 | 0.0143 | 0.9500 | 1.0527 |
| Brain | 0.0144 | 0.0088 | 1.6450 | 0.6079 |
| Hematopoietic | 0.0070 | 0.0000 | undef | 0.0000 |
| Skin | 0.0000 | 0.0000 | undef | undef |
| Hepatic | 0.0099 | 0.0000 | undef | 0.0000 |
| Heart | 0.0042 | 0.0137 | 0.3083 | 3.2436 |
| Testicles | 0.0000 | 0.0117 | 0.0000 | undef |
| Lungs | 0.0062 | 0.0095 | 0.6584 | 1.5189 |
| Stomach-esophagus | 0.0000 | 0.0997 | 0.0000 | undef |
| Muscle-skeleton | 0.0017 | 0.0180 | 0.0952 | 10.5076 |
| Kidneys | 0.0119 | 0.0137 | 0.8683 | 1.1517 |
| Pancreas | 0.0095 | 0.0110 | 0.8571 | 1.1667 |
| Penis | 0.0150 | 0.0000 | undef | 0.0000 |
| Prostate | 0.0071 | 0.0128 | 0.5593 | 1.7879 |
| Uterus | 0.0099 | 0.0071 | 1.3925 | 0.7181 |
| Breast hyperplasia | 0.0036 | | | |
| Small intestine | 0.0218 | | | |
| Prostatic hyperplasia | 0.0089 | | | |
| Seminal vesicles | 0.0089 | | | |
| Sensory organs | 0.0000 | | | |
| White blood cells | 0.0070 | | | |
| | FETUS | | | |
| Development | 0.0000 | | | |
| Gastrointestinal | 0.0031 | | | |
| Brain | 0.0000 | | | |
| Hematopoietic | 0.0157 | | | |
| Heart-blood vessels | 0.0000 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0068 | | | |
| Ovary-uterus | 0.0068 | | | |
| Endocrine tissue | 0.0000 | | | |
| Fetal | 0.0029 | | | |
| Gastrointestinal | 0.0366 | | | |
| Hematopoietic | 0.0171 | | | |
| Skin-muscle | 0.0032 | | | |
| Testicles | 0.0078 | | | |
| Lungs | 0.0082 | | | |
| Nerves | 0.0080 | | | |
| Prostate | 0.0064 | | | |
| Sensory organs | 0.0077 | | | |

Electronic Northern for SEQ. ID NO.: 56

| | | | | |
|---|---|---|---|---|
| | NORMAL | | | |
| Bladder | 0.0000 | 0.0204 | 0.0000 | undef |
| Breast | 0.0000 | 0.0218 | 0.0000 | undef |
| Ovary | 0.0000 | 0.0026 | 0.0000 | undef |
| Endocrine tissue | 0.0000 | 0.0054 | 0.0000 | undef |
| Gastrointestinal | 0.0039 | 0.0048 | 0.8143 | 1.2281 |
| Brain | 0.0008 | 0.0011 | 0.7741 | 1.2918 |
| Hematopoietic | 0.0000 | 0.0000 | undef | undef |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0012 | 0.0047 | 0.2634 | 3.7971 |
| Stomach-esophagus | 0.0000 | 0.0000 | undef | undef |
| Muscle-skeleton | 0.0000 | 0.0000 | undef | undef |
| Kidneys | 0.0000 | 0.0000 | undef | undef |
| Pancreas | 0.0000 | 0.0110 | 0.0000 | undef |
| Penis | 0.0000 | 0.0267 | 0.0000 | undef |
| Prostate | 0.0000 | 0.0000 | undef | undef |
| Uterus | 0.0033 | 0.0000 | undef | 0.0000 |
| Breast hyperplasia | 0.0000 | | | |
| Small intestine | 0.0000 | | | |
| Prostatic hyperplasia | 0.0000 | | | |
| Seminal vesicles | 0.0000 | | | |
| Sensory organs | 0.0000 | | | |
| White blood cells | 0.0009 | | | |
| | FETUS | | | |
| Development | 0.0000 | | | |
| Gastrointestinal | 0.0062 | | | |
| Brain | 0.0000 | | | |
| Hematopoietic | 0.0079 | | | |
| Heart-blood vessels | 0.0000 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0136 | | | |
| Ovary-uterus | 0.0320 | | | |
| Endocrine tissue | 0.0000 | | | |
| Fetal | 0.0367 | | | |
| Gastrointestinal | 0.0000 | | | |
| Hematopoietic | 0.0114 | | | |
| Skin-muscle | 0.0065 | | | |
| Testicles | 0.0000 | | | |
| Lungs | 0.0164 | | | |
| Nerves | 0.0000 | | | |
| Prostate | 0.0000 | | | |
| Sensory organs | 0.0000 | | | |

Electronic Northern for SEQ. ID NO.: 57

| | | | | |
|---|---|---|---|---|
| | NORMAL | | | |
| Bladder | 0.0046 | 0.0051 | 0.9092 | 1.0998 |
| Breast | 0.0027 | 0.0109 | 0.2446 | 4.0878 |
| Ovary | 0.0091 | 0.0026 | 3.5059 | 0.2852 |
| Endocrine tissue | 0.0036 | 0.0054 | 0.6698 | 1.4930 |
| Gastrointestinal | 0.0019 | 0.0000 | undef | 0.0000 |
| Brain | 0.0017 | 0.0044 | 0.3871 | 2.5836 |
| Hematopoietic | 0.0028 | 0.0000 | undef | 0.0000 |
| Skin | 0.0000 | 0.0847 | 0.0000 | undef |
| Hepatic | 0.0000 | 0.0000 | undef | undef |
| Heart | 0.0021 | 0.0000 | undef | 0.0000 |
| Testicles | 0.0000 | 0.0000 | undef | undef |
| Lungs | 0.0050 | 0.0000 | undef | 0.0000 |
| Stomach-esophagus | 0.0000 | 0.0000 | undef | undef |
| Muscle-skeleton | 0.0017 | 0.0060 | 0.2855 | 3.5025 |
| Kidneys | 0.0000 | 0.0000 | undef | undef |
| Pancreas | 0.0057 | 0.0110 | 0.5143 | 1.9446 |
| Penis | 0.0000 | 0.0000 | undef | undef |
| Prostate | 0.0000 | 0.0021 | 0.0000 | undef |
| Uterus | 0.0033 | 0.0000 | undef | 0.0000 |
| Breast hyperplasia | 0.0000 | | | |
| Small intestine | 0.0031 | | | |
| Prostatic hyperplasia | 0.0059 | | | |
| Seminal vesicles | 0.0000 | | | |
| Sensory organs | 0.0000 | | | |
| White blood cells | 0.0061 | | | |
| | FETUS | | | |
| Development | 0.0000 | | | |
| Gastrointestinal | 0.0031 | | | |
| Brain | 0.0000 | | | |
| Hematopoietic | 0.0079 | | | |
| Heart-blood vessels | 0.0000 | | | |
| Lungs | 0.0074 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0000 | | | |
| Endocrine tissue | 0.0000 | | | |
| Fetal | 0.0000 | | | |
| Gastrointestinal | 0.0122 | | | |
| Hematopoietic | 0.0000 | | | |
| Skin-muscle | 0.0000 | | | |
| Testicles | 0.0000 | | | |
| Lungs | 0.0000 | | | |
| Nerves | 0.0050 | | | |
| Prostate | 0.0128 | | | |
| Sensory organs | 0.0000 | | | |

Electronic Northern for SEQ. ID NO.: 58

| | | | | |
|---|---|---|---|---|
| | NORMAL | | | |
| Bladder | 0.0000 | 0.0000 | undef | undef |
| Breast | 0.0173 | 0.0523 | 0.3313 | 3.0187 |
| Ovary | 0.0000 | 0.0026 | 0.0000 | undef |
| Endocrine tissue | 0.0018 | 0.0000 | undef | 0.0000 |
| Gastrointestinal | 0.0000 | 0.0000 | undef | undef |
| Brain | 0.0000 | 0.0000 | undef | undef |
| Hematopoietic | 0.0028 | 0.0000 | undef | 0.0000 |
| Skin | 0.0050 | 0.0000 | undef | 0.0000 |
| Hepatic | 0.0000 | 0.0000 | undef | undef |
| Heart | 0.0000 | 0.0000 | undef | undef |
| Testicles | 0.0000 | 0.0000 | undef | undef |
| Lungs | 0.0000 | 0.0000 | undef | undef |
| Stomach-esophagus | 0.0000 | 0.0000 | undef | undef |
| Muscle-skeleton | 0.0000 | 0.0000 | undef | undef |
| Kidneys | 0.0000 | 0.0000 | undef | undef |
| Pancreas | 0.0000 | 0.0000 | undef | undef |
| Penis | 0.0000 | 0.0000 | undef | undef |
| Prostate | 0.0024 | 0.0000 | undef | 0.0000 |
| Uterus | 0.0066 | 0.0285 | 0.2321 | 4.3088 |
| Breast hyperplasia | 0.0073 | | | |
| Small intestine | 0.0000 | | | |
| Prostatic hyperplasia | 0.0000 | | | |
| Seminal vesicles | 0.0000 | | | |
| Sensory organs | 0.0000 | | | |
| White blood cells | 0.0000 | | | |
| | FETUS | | | |
| Development | 0.0000 | | | |
| Gastrointestinal | 0.0000 | | | |
| Brain | 0.0000 | | | |
| Hematopoietic | 0.0000 | | | |
| Heart-blood vessels | 0.0000 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0000 | | | |
| Endocrine tissue | 0.0000 | | | |
| Fetal | 0.0000 | | | |
| Gastrointestinal | 0.0000 | | | |
| Hematopoietic | 0.0000 | | | |
| Skin-muscle | 0.0000 | | | |
| Testicles | 0.0000 | | | |
| Lungs | 0.0000 | | | |
| Nerves | 0.0000 | | | |
| Prostate | 0.0128 | | | |
| Sensory organs | 0.0000 | | | |

Electronic Northern for SEQ. ID NO.: 59

| | | | | |
|---|---|---|---|---|
| | NORMAL | | | |
| Bladder | 0.0046 | 0.0051 | 0.9092 | 1.0998 |
| Breast | 0.0013 | 0.0109 | 0.1223 | 8.1755 |
| Ovary | 0.0061 | 0.0000 | undef | 0.0000 |
| Endocrine tissue | 0.0018 | 0.0000 | undef | 0.0000 |
| Gastrointestinal | 0.0039 | 0.0095 | 0.4071 | 2.4562 |
| Brain | 0.0025 | 0.0066 | 0.3871 | 2.5836 |
| Hematopoietic | 0.0000 | 0.0000 | undef | undef |
| Skin | 0.0000 | 0.0000 | undef | undef |
| Hepatic | 0.0000 | 0.0065 | 0.0000 | undef |
| Heart | 0.0074 | 0.0000 | undef | 0.0000 |
| Testicles | 0.0061 | 0.0234 | 0.2612 | 3.8288 |
| Lungs | 0.0025 | 0.0095 | 0.2634 | 3.7971 |
| Stomach-esophagus | 0.0000 | 0.0000 | undef | undef |
| Muscle-skeleton | 0.0017 | 0.0000 | undef | 0.0000 |
| Kidneys | 0.0030 | 0.0068 | 0.4342 | 2.3033 |
| Pancreas | 0.0000 | 0.0055 | 0.0000 | undef |
| Penis | 0.0060 | 0.0000 | undef | 0.0000 |
| Prostate | 0.0024 | 0.0000 | undef | 0.0000 |
| Uterus | 0.0017 | 0.0000 | undef | 0.0000 |
| Breast hyperplasia | 0.0073 | | | |
| Small intestine | 0.0031 | | | |
| Prostatic hyperplasia | 0.0089 | | | |
| Seminal vesicles | 0.0089 | | | |
| Sensory organs | 0.0000 | | | |
| White blood cells | 0.0009 | | | |
| | FETUS | | | |
| Development | 0.0000 | | | |
| Gastrointestinal | 0.0000 | | | |
| Brain | 0.0000 | | | |
| Hematopoietic | 0.0000 | | | |
| Heart-blood vessels | 0.0082 | | | |
| Lungs | 0.0037 | | | |
| Kidneys | 0.0185 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0068 | | | |
| Ovary-uterus | 0.0160 | | | |
| Endocrine tissue | 0.0245 | | | |
| Fetal | 0.0035 | | | |
| Gastrointestinal | 0.0000 | | | |
| Hematopoietic | 0.0114 | | | |
| Skin-muscle | 0.0000 | | | |
| Testicles | 0.0000 | | | |
| Lungs | 0.0000 | | | |
| Nerves | 0.0020 | | | |
| Prostate | 0.0064 | | | |
| Sensory organs | 0.0155 | | | |

Electronic Northern for SEQ. ID NO.: 61

| | | | | |
|---|---|---|---|---|
| | NORMAL | | | |
| Bladder | 0.0000 | 0.0000 | undef | undef |
| Breast | 0.0013 | 0.0109 | 0.1223 | 8.1755 |
| Ovary | 0.0000 | 0.0000 | undef | undef |
| Endocrine tissue | 0.0091 | 0.0245 | 0.3721 | 2.6874 |
| Gastrointestinal | 0.0000 | 0.0000 | undef | undef |
| Brain | 0.0017 | 0.0022 | 0.7741 | 1.2918 |
| Hematopoietic | 0.0014 | 0.0000 | undef | 0.0000 |
| Skin | 0.0000 | 0.0000 | undef | undef |
| Hepatic | 0.0000 | 0.0000 | undef | undef |
| Heart | 0.0032 | 0.0000 | undef | 0.0000 |
| Testicles | 0.0061 | 0.0000 | undef | 0.0000 |
| Lungs | 0.0050 | 0.0024 | 2.1069 | 0.4746 |
| Stomach-esophagus | 0.0000 | 0.0000 | undef | undef |
| Muscle-skeleton | 0.0034 | 0.0000 | undef | 0.0000 |
| Kidneys | 0.0030 | 0.0000 | undef | 0.0000 |
| Pancreas | 0.0000 | 0.0055 | 0.0000 | undef |
| Penis | 0.0030 | 0.0000 | undef | 0.0000 |
| Prostate | 0.0024 | 0.0085 | 0.2797 | 3.5758 |
| Uterus | 0.0000 | 0.0000 | undef | undef |
| Breast hyperplasia | 0.0000 | | | |
| Small intestine | 0.0031 | | | |
| Prostatic hyperplasia | 0.0059 | | | |
| Seminal vesicles | 0.0000 | | | |
| Sensory organs | 0.0000 | | | |
| White blood cells | 0.0000 | | | |
| | FETUS | | | |
| Development | 0.0000 | | | |
| Gastrointestinal | 0.0031 | | | |
| Brain | 0.0000 | | | |
| Hematopoietic | 0.0157 | | | |
| Heart-blood vessels | 0.0041 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0068 | | | |
| Endocrine tissue | 0.0000 | | | |
| Fetal | 0.0128 | | | |
| Gastrointestinal | 0.0000 | | | |
| Hematopoietic | 0.0000 | | | |
| Skin-muscle | 0.0162 | | | |
| Testicles | 0.0000 | | | |
| Lungs | 0.0082 | | | |
| Nerves | 0.0020 | | | |
| Prostate | 0.0000 | | | |
| Sensory organs | 0.0000 | | | |

Electronic Northern for SEQ. ID NO.: 62

| | | | | |
|---|---|---|---|---|
| | NORMAL | | | |
| Bladder | 0.0000 | 0.0051 | 0.0000 | undef |
| Breast | 0.0080 | 0.0153 | 0.5242 | 1.9076 |
| Ovary | 0.0091 | 0.0078 | 1.1686 | 0.8557 |
| Endocrine tissue | 0.0055 | 0.0109 | 0.5023 | 1.9907 |
| Gastrointestinal | 0.0078 | 0.0238 | 0.3257 | 3.0703 |
| Brain | 0.0051 | 0.0077 | 0.6635 | 1.5071 |
| Hematopoietic | 0.0140 | 0.0000 | undef | 0.0000 |
| Skin | 0.0000 | 0.0000 | undef | undef |
| Hepatic | 0.0099 | 0.0000 | undef | 0.0000 |
| Heart | 0.0000 | 0.0000 | undef | undef |
| Testicles | 0.0061 | 0.0000 | undef | 0.0000 |
| Lungs | 0.0050 | 0.0047 | 1.0534 | 0.9493 |
| Stomach-esophagus | 0.0000 | 0.0077 | 0.0000 | undef |
| Muscle-skeleton | 0.0017 | 0.0000 | undef | 0.0000 |
| Kidneys | 0.0059 | 0.0068 | 0.8683 | 1.1517 |
| Pancreas | 0.0019 | 0.0000 | undef | 0.0000 |
| Penis | 0.0030 | 0.0000 | undef | 0.0000 |
| Prostate | 0.0048 | 0.0021 | 2.2373 | 0.4470 |
| Uterus | 0.0033 | 0.0214 | 0.1547 | 6.4632 |
| Breast hyperplasia | 0.0000 | | | |
| Small intestine | 0.0031 | | | |
| Prostatic hyperplasia | 0.0119 | | | |
| Seminal vesicles | 0.0000 | | | |
| Sensory organs | 0.0000 | | | |
| White blood cells | 0.0035 | | | |
| | FETUS | | | |
| Development | 0.0000 | | | |
| Gastrointestinal | 0.0000 | | | |
| Brain | 0.0063 | | | |
| Hematopoietic | 0.0079 | | | |
| Heart-blood vessels | 0.0041 | | | |
| Lungs | 0.0074 | | | |
| Kidneys | 0.0185 | | | |
| Prostate | 0.0000 | | | |
| Sensory organs | 0.0140 | | | |
| | STANDARDIZED/ | | | |
| | SUBTRACTED | | | |
| | LIBRARIES | | | |
| Breast | 0.0000 | | | |
| Ovary-uterus | 0.0091 | | | |
| Endocrine tissue | 0.0000 | | | |
| Fetal | 0.0087 | | | |
| Gastrointestinal | 0.0244 | | | |
| Hematopoietic | 0.0057 | | | |
| Skin-muscle | 0.0097 | | | |
| Testicles | 0.0156 | | | |
| Lungs | 0.0000 | | | |
| Nerves | 0.0030 | | | |
| Prostate | 0.0000 | | | |
| Sensory organs | 0.0697 | | | |

Electronic Northern for SEQ. ID NO.: 63

| | | | | |
|---|---|---|---|---|
| | NORMAL | | | |
| Bladder | 0.0046 | 0.0000 | undef | 0.0000 |
| Breast | 0.0000 | 0.0109 | 0.0000 | undef |
| Ovary | 0.0030 | 0.0026 | 1.1686 | 0.8557 |
| Endocrine tissue | 0.0018 | 0.0027 | 0.6698 | 1.4930 |
| Gastrointestinal | 0.0039 | 0.0000 | undef | 0.0000 |
| Brain | 0.0034 | 0.0044 | 0.7741 | 1.2918 |
| Hematopoietic | 0.0042 | 0.0000 | undef | 0.0000 |
| Skin | 0.0000 | 0.0000 | undef | undef |
| Hepatic | 0.0149 | 0.0000 | undef | 0.0000 |
| Heart | 0.0011 | 0.0000 | undef | 0.0000 |
| Testicles | 0.0000 | 0.0000 | undef | undef |
| Lungs | 0.0012 | 0.0000 | undef | 0.0000 |
| Stomach-esophagus | 0.0000 | 0.0000 | undef | undef |
| Muscle-skeleton | 0.0017 | 0.0060 | 0.2855 | 3.5025 |
| Kidneys | 0.0059 | 0.0000 | undef | 0.0000 |
| Pancreas | 0.0019 | 0.0055 | 0.3428 | 2.9168 |
| Penis | 0.0060 | 0.0000 | undef | 0.0000 |
| Prostate | 0.0000 | 0.0000 | undef | undef |
| Uterus | 0.0033 | 0.0000 | undef | 0.0000 |
| Breast hyperplasia | 0.0036 | | | |
| Small intestine | 0.0000 | | | |
| Prostatic hyperplasia | 0.0000 | | | |
| Seminal vesicles | 0.0089 | | | |
| Sensory organs | 0.0000 | | | |
| White blood cells | 0.0026 | | | |
| | FETUS | | | |
| Development | 0.0000 | | | |
| Gastrointestinal | 0.0092 | | | |
| Brain | 0.0125 | | | |
| Hematopoietic | 0.0039 | | | |
| Heart-blood vessels | 0.0082 | | | |
| Lungs | 0.0000 | | | |
| Kidneys | 0.0062 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0046 | | | |
| Endocrine tissue | 0.0490 | | | |
| Fetal | 0.0058 | | | |
| Gastrointestinal | 0.0000 | | | |
| Hematopoietic | 0.0000 | | | |
| Skin-muscle | 0.0065 | | | |
| Testicles | 0.0000 | | | |
| Lungs | 0.0000 | | | |
| Nerves | 0.0010 | | | |
| Prostate | 0.0000 | | | |
| Sensory organs | 0.0000 | | | |

Electronic Northern for SEQ. ID NO.: 64

| | | | | |
|---|---|---|---|---|
| | NORMAL | | | |
| Bladder | 0.0046 | 0.0665 | 0.0699 | 14.2976 |
| Breast | 0.0080 | 0.0436 | 0.1835 | 5.4504 |
| Ovary | 0.0061 | 0.0234 | 0.2597 | 3.8507 |
| Endocrine tissue | 0.0000 | 0.0000 | undef | undef |
| Gastrointestinal | 0.0213 | 0.0571 | 0.3732 | 2.6795 |
| Brain | 0.0008 | 0.0000 | undef | 0.0000 |
| Hematopoietic | 0.0028 | 0.0000 | undef | 0.0000 |
| Skin | 0.0000 | 0.0000 | undef | undef |
| Hepatic | 0.0000 | 0.0194 | 0.0000 | undef |
| Heart | 0.0000 | 0.0000 | undef | undef |
| Testicles | 0.0000 | 0.0000 | undef | undef |
| Lungs | 0.0087 | 0.0142 | 0.6145 | 1.6273 |
| Stomach-esophagus | 0.0290 | 0.0000 | undef | 0.0000 |
| Muscle-skeleton | 0.0000 | 0.0000 | undef | undef |
| Kidneys | 0.0119 | 0.0205 | 0.5789 | 1.7275 |
| Pancreas | 0.0133 | 0.0276 | 0.4800 | 2.0835 |
| Penis | 0.0000 | 0.0000 | undef | undef |
| Prostate | 0.0119 | 0.0106 | 1.1186 | 0.8939 |
| Uterus | 0.0033 | 0.0071 | 0.4642 | 2.1544 |
| Breast hyperplasia | 0.0073 | | | |
| Small intestine | 0.0343 | | | |
| Prostatic hyperplasia | 0.0208 | | | |
| Seminal vesicles | 0.0178 | | | |
| Sensory organs | 0.0353 | | | |
| White blood cells | 0.0000 | | | |
| | FETUS | | | |
| Development | 0.0000 | | | |
| Gastrointestinal | 0.0092 | | | |
| Brain | 0.0000 | | | |
| Hematopoietic | 0.0000 | | | |
| Heart-blood vessels | 0.0000 | | | |
| Lungs | 0.0037 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0476 | | | |
| Ovary-uterus | 0.0274 | | | |
| Endocrine tissue | 0.0000 | | | |
| Fetal | 0.0052 | | | |
| Gastrointestinal | 0.0122 | | | |
| Hematopoietic | 0.0000 | | | |
| Skin-muscle | 0.0000 | | | |
| Testicles | 0.0000 | | | |
| Lungs | 0.0082 | | | |
| Nerves | 0.0000 | | | |
| Prostate | 0.0064 | | | |
| Sensory organs | 0.0000 | | | |

Electronic Northern for SEQ. ID NO.: 65

| | | | | |
|---|---|---|---|---|
| | NORMAL | | | |
| Bladder | 0.0000 | 0.0051 | 0.0000 | undef |
| Breast | 0.0053 | 0.0174 | 0.3058 | 3.2702 |
| Ovary | 0.0061 | 0.0026 | 2.3372 | 0.4279 |
| Endocrine tissue | 0.0073 | 0.0027 | 2.6791 | 0.3733 |
| Gastrointestinal | 0.0097 | 0.0048 | 2.0357 | 0.4912 |
| Brain | 0.0059 | 0.0055 | 1.0837 | 0.9227 |
| Hematopoietic | 0.0042 | 0.0000 | undef | 0.0000 |
| Skin | 0.0050 | 0.0000 | undef | 0.0000 |
| Hepatic | 0.0000 | 0.0000 | undef | undef |
| Heart | 0.0053 | 0.0000 | undef | 0.0000 |
| Testicles | 0.0000 | 0.0000 | undef | undef |
| Lungs | 0.0087 | 0.0071 | 1.2290 | 0.8137 |
| Stomach-esophagus | 0.0000 | 0.0153 | 0.0000 | undef |
| Muscle-skeleton | 0.0103 | 0.0000 | undef | 0.0000 |
| Kidneys | 0.0000 | 0.0068 | 0.0000 | undef |
| Pancreas | 0.0038 | 0.0055 | 0.6857 | 1.4584 |
| Penis | 0.0120 | 0.0267 | 0.4492 | 2.2260 |
| Prostate | 0.0000 | 0.0021 | 0.0000 | undef |
| Uterus | 0.0099 | 0.0142 | 0.6963 | 1.4363 |
| Breast hyperplasia | 0.0000 | | | |
| Small intestine | 0.0000 | | | |
| Prostatic hyperplasia | 0.0000 | | | |
| Seminal vesicles | 0.0000 | | | |
| Sensory organs | 0.0000 | | | |
| White blood cells | 0.0087 | | | |
| | FETUS | | | |
| Development | 0.0000 | | | |
| Gastrointestinal | 0.0154 | | | |
| Brain | 0.0000 | | | |
| Hematopoietic | 0.0039 | | | |
| Heart-blood vessels | 0.0041 | | | |
| Lungs | 0.0000 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0136 | | | |
| Ovary-uterus | 0.0046 | | | |
| Endocrine tissue | 0.0000 | | | |
| Fetal | 0.0082 | | | |
| Gastrointestinal | 0.0000 | | | |
| Hematopoietic | 0.0000 | | | |
| Skin-muscle | 0.0032 | | | |
| Testicles | 0.0156 | | | |
| Lungs | 0.0082 | | | |
| Nerves | 0.0040 | | | |
| Prostate | 0.0128 | | | |
| Sensory organs | 0.0000 | | | |

Electronic Northern for SEQ. ID NO.: 66

| | | | | |
|---|---|---|---|---|
| | NORMAL | | | |
| Bladder | 0.0093 | 0.1508 | 0.0616 | 16.2223 |
| Breast | 0.0013 | 0.0392 | 0.0340 | 29.4320 |
| Ovary | 0.0152 | 0.0000 | undef | 0.0000 |
| Endocrine tissue | 0.0018 | 0.0027 | 0.6698 | 1.4930 |
| Gastrointestinal | 0.0174 | 0.0048 | 3.6642 | 0.2729 |
| Brain | 0.0051 | 0.0055 | 0.9289 | 1.0765 |
| Hematopoietic | 0.0993 | 0.0000 | undef | 0.0000 |
| Skin | 0.0249 | 0.0000 | undef | 0.0000 |
| Hepatic | 0.0000 | 0.0129 | 0.0000 | undef |
| Heart | 0.0053 | 0.0000 | undef | 0.0000 |
| Testicles | 0.0061 | 0.0000 | undef | 0.0000 |
| Lungs | 0.0261 | 0.0307 | 0.8508 | 1.1753 |
| Stomach-esophagus | 0.1062 | 0.0230 | 4.6197 | 0.2165 |
| Muscle-skeleton | 0.0171 | 0.0180 | 0.9517 | 1.0508 |
| Kidneys | 0.0000 | 0.0068 | 0.0000 | undef |
| Pancreas | 0.0114 | 0.0000 | undef | 0.0000 |
| Penis | 0.1258 | 0.1600 | 0.7862 | 1.2720 |
| Prostate | 0.0000 | 0.0000 | undef | undef |
| Uterus | 0.0826 | 0.0000 | undef | 0.0000 |
| Breast hyperplasia | 0.0036 | | | |
| Small intestine | 0.0000 | | | |
| Prostatic hyperplasia | 0.0000 | | | |
| Seminal vesicles | 0.0178 | | | |
| Sensory organs | 0.0000 | | | |
| White blood cells | 0.1018 | | | |
| | FETUS | | | |
| Development | 0.0000 | | | |
| Gastrointestinal | 0.0062 | | | |
| Brain | 0.0000 | | | |
| Hematopoietic | 0.0000 | | | |
| Heart-blood vessels | 0.0000 | | | |
| Lungs | 0.0037 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0000 | | | |
| Endocrine tissue | 0.0000 | | | |
| Fetal | 0.0052 | | | |
| Gastrointestinal | 0.0122 | | | |
| Hematopoietic | 0.0057 | | | |
| Skin-muscle | 0.0000 | | | |
| Testicles | 0.0000 | | | |
| Lungs | 0.0410 | | | |
| Nerves | 0.0000 | | | |
| Prostate | 0.0449 | | | |
| Sensory organs | 0.0000 | | | |

Electronic Northern for SEQ. ID NO.: 67

| | | | | |
|---|---|---|---|---|
| | NORMAL | | | |
| Bladder | 0.0093 | 0.0051 | 1.8185 | 0.5499 |
| Breast | 0.0013 | 0.0044 | 0.3058 | 3.2702 |
| Ovary | 0.0000 | 0.0052 | 0.0000 | undef |
| Endocrine tissue | 0.0073 | 0.0109 | 0.6698 | 1.4930 |
| Gastrointestinal | 0.0000 | 0.0000 | undef | undef |
| Brain | 0.0068 | 0.0055 | 1.2386 | 0.8074 |
| Hematopoietic | 0.0028 | 0.0000 | undef | 0.0000 |
| Skin | 0.0000 | 0.0000 | undef | undef |
| Hepatic | 0.0000 | 0.0129 | 0.0000 | undef |
| Heart | 0.0042 | 0.0000 | undef | 0.0000 |
| Testicles | 0.0000 | 0.0234 | 0.0000 | undef |
| Lungs | 0.0025 | 0.0000 | undef | 0.0000 |
| Stomach-esophagus | 0.0000 | 0.0000 | undef | undef |
| Muscle-skeleton | 0.0000 | 0.0000 | undef | undef |
| Kidneys | 0.0000 | 0.0068 | 0.0000 | undef |
| Pancreas | 0.0057 | 0.0000 | undef | 0.0000 |
| Penis | 0.0030 | 0.0000 | undef | 0.0000 |
| Prostate | 0.0786 | 0.0554 | 1.4198 | 0.7043 |
| Uterus | 0.0033 | 0.0000 | undef | 0.0000 |
| Breast hyperplasia | 0.0000 | | | |
| Small intestine | 0.0000 | | | |
| Prostatic hyperplasia | 0.1011 | | | |
| Seminal vesicles | 0.0000 | | | |
| Sensory organs | 0.0000 | | | |
| White blood cells | 0.0017 | | | |
| | FETUS | | | |
| Development | 0.0000 | | | |
| Gastrointestinal | 0.0062 | | | |
| Brain | 0.0000 | | | |
| Hematopoietic | 0.0039 | | | |
| Heart-blood vessels | 0.0000 | | | |
| Lungs | 0.0037 | | | |
| Kidneys | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0000 | | | |
| Endocrine tissue | 0.0000 | | | |
| Fetal | 0.0082 | | | |
| Gastrointestinal | 0.0000 | | | |
| Hematopoietic | 0.0057 | | | |
| Skin-muscle | 0.0065 | | | |
| Testicles | 0.0078 | | | |
| Lungs | 0.0082 | | | |
| Nerves | 0.0030 | | | |
| Prostate | 0.0128 | | | |
| Sensory organs | 0.0000 | | | |

Electronic Northern for Seq. ID: 149

| | | | | |
|---|---|---|---|---|
| | NORMAL | | | |
| B lymphoma | 0.0050 | 0.0136 | 0.3679 | 2.7181 |
| Bladder | 0.0078 | 0.0258 | 0.3017 | 3.3143 |
| Breast | 0.0114 | 0.0225 | 0.5088 | 1.9654 |
| Large intestine | 0.0115 | 0.0085 | 1.3456 | 0.7432 |
| Small intestine | 0.0192 | 0.0107 | 1.8036 | 0.5545 |
| Ovary | 0.0089 | 0.0095 | 0.9333 | 1.0715 |
| Endocrine tissue | 0.0209 | 0.0337 | 0.6195 | 1.6143 |
| Brain | 0.0203 | 0.0170 | 1.1947 | 0.8371 |
| Skin | 0.0367 | 0.0000 | undef | 0.0000 |
| Hepatic | 0.0186 | 0.0254 | 0.7324 | 1.3653 |
| Heart | 0.0183 | 0.0137 | 1.3291 | 0.7524 |
| Testicles | 0.0241 | 0.0000 | undef | 0.0000 |
| Lung | 0.0146 | 0.0166 | 0.8770 | 1.1402 |
| Stomach-esophagus | 0.0290 | 0.0128 | 2.2671 | 0.4411 |
| Muscle-skeleton | 0.0069 | 0.0074 | 0.9278 | 1.0778 |
| Kidney | 0.0157 | 0.0048 | 3.2497 | 0.3077 |
| Pancreas | 0.0050 | 0.0221 | 0.2244 | 4.4570 |
| Prostate | 0.0217 | 0.0169 | 1.2801 | 0.7812 |
| T lymphoma | 0.0076 | 0.0000 | undef | 0.0000 |
| Uterus | 0.0163 | 0.0230 | 0.7068 | 1.4148 |
| White blood cells | 0.0267 | 0.0000 | undef | 0.0000 |
| Hematopoietic | 0.0080 | | | |
| Penis | 0.0161 | | | |
| Seminal vesicle | 0.0281 | | | |
| Sensory organs | 0.0118 | | | |
| | FETUS | | | |
| Development | 0.0000 | | | |
| Gastrointestinal | 0.0111 | | | |
| Brain | 0.0063 | | | |
| Hematopoietic | 0.0000 | | | |
| Skin | 0.0000 | | | |
| Hepatic | 0.0000 | | | |
| Heart-blood vessels | 0.0107 | | | |
| Lung | 0.0108 | | | |
| Adrenal gland | 0.0000 | | | |
| Kidney | 0.0124 | | | |
| Placenta | 0.0303 | | | |
| Prostate | 0.0499 | | | |
| Sensory organs | 0.0000 | | | |
| | STANDARDIZED/ | | | |
| | SUBTRACTED | | | |
| | LIBRARIES | | | |
| Breast | 0.0000 | | | |
| Breast_t | 0.0000 | | | |
| Large intestine_t | 0.0000 | | | |
| Ovary_n | 0.0000 | | | |
| Ovary_t | 0.0253 | | | |
| Endocrine tissue | 0.0000 | | | |
| Fetal | 0.0070 | | | |
| Gastrointestinal | 0.0366 | | | |
| Hematopoietic | 0.0000 | | | |
| Skin-muscle | 0.0227 | | | |
| Testicles_n | 0.0042 | | | |
| Testicles_t | 0.0000 | | | |
| Lungs_n | 0.0000 | | | |
| Lungs_t | 0.0000 | | | |
| Nerves | 0.0090 | | | |
| Kidney_t | 0.0000 | | | |
| Ovary_uterus | 0.0203 | | | |
| Prostate_n | 0.0121 | | | |
| Sensory organs | 0.0000 | | | |
| White blood cells | 0.0000 | | | |

Electronic Northern for Seq. ID: 150

| | | | | |
|---|---|---|---|---|
| | NORMAL | | | |
| B lymphoma | 0.0050 | 0.0000 | undef | 0.0000 |
| Bladder | 0.0078 | 0.0117 | 0.6638 | 1.5065 |
| Breast | 0.0088 | 0.0155 | 0.5693 | 1.7566 |
| Large intestine | 0.0211 | 0.0028 | 7.4006 | 0.1351 |
| Small intestine | 0.0027 | 0.0000 | undef | 0.0000 |
| Ovary | 0.0119 | 0.0024 | 4.9773 | 0.2009 |
| Endocrine tissue | 0.0032 | 0.0142 | 0.2263 | 4.4181 |
| Brain | 0.0035 | 0.0000 | undef | 0.0000 |
| Skin | 0.0257 | 0.0000 | undef | 0.0000 |
| Hepatic | 0.0279 | 0.0000 | undef | 0.0000 |
| Heart | 0.0071 | 0.0137 | 0.5169 | 1.9347 |
| Testicles | 0.0040 | 0.0000 | undef | 0.0000 |
| Lung | 0.0292 | 0.0148 | 1.9733 | 0.5068 |
| Stomach-esophagus | 0.0000 | 0.0064 | 0.0000 | undef |
| Muscle-skeleton | 0.0017 | 0.0000 | undef | 0.0000 |
| Kidney | 0.0269 | 0.0000 | undef | 0.0000 |
| Pancreas | 0.0165 | 0.0000 | undef | 0.0000 |
| Prostate | 0.0141 | 0.0052 | 2.7132 | 0.3686 |
| T lymphoma | 0.0000 | 0.0000 | undef | undef |
| Uterus | 0.0044 | 0.0000 | undef | 0.0000 |
| White blood cells | 0.0000 | 0.0000 | undef | undef |
| Hematopoietic | 0.0040 | | | |
| Penis | 0.0000 | | | |
| Seminal vesicle | 0.0070 | | | |
| Sensory organs | 0.0000 | | | |
| | FETUS | | | |
| Development | 0.0000 | | | |
| Gastrointestinal | 0.0056 | | | |
| Brain | 0.0000 | | | |
| Hematopoietic | 0.0039 | | | |
| Skin | 0.0000 | | | |
| Hepatic | 0.0000 | | | |
| Heart-blood vessel | 0.0036 | | | |
| Lung | 0.0145 | | | |
| Adrenal gland | 0.0254 | | | |
| Kidney | 0.0000 | | | |
| Placenta | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Breast_t | 0.0000 | | | |
| Large intestine_t | 0.0000 | | | |
| Ovary_n | 0.0000 | | | |
| Ovary_t | 0.0000 | | | |
| Endocrine tissue | 0.0000 | | | |
| Fetal | 0.0058 | | | |
| Gastrointestinal | 0.0244 | | | |
| Hematopoietic | 0.0000 | | | |
| Skin-muscle | 0.0130 | | | |
| Testicles_n | 0.0000 | | | |
| Testicles_t | 0.0000 | | | |
| Lungs_n | 0.0293 | | | |
| Lungs_t | 0.0000 | | | |
| Nerves | 0.0020 | | | |
| Kidney_t | 0.0000 | | | |
| Ovary_uterus | 0.0068 | | | |
| Prostate_n | 0.0243 | | | |
| Sensory organs | 0.0000 | | | |
| White blood cells | 0.0000 | | | |

Electronic Northern for Seq. ID: 151

| | | | | |
|---|---|---|---|---|
| | NORMAL | | | |
| B lymphoma | 0.0050 | 0.0000 | undef | 0.0000 |
| Bladder | 0.0000 | 0.0070 | 0.0000 | undef |
| Breast | 0.0070 | 0.0098 | 0.7157 | 1.3973 |
| Large intestine | 0.0038 | 0.0028 | 1.3456 | 0.7432 |
| Small intestine | 0.0082 | 0.0107 | 0.7730 | 1.2937 |
| Ovary | 0.0030 | 0.0119 | 0.2489 | 4.0182 |
| Endocrine tissue | 0.0048 | 0.0071 | 0.6790 | 1.4727 |
| Brain | 0.0104 | 0.0120 | 0.8704 | 1.1489 |
| Skin | 0.0037 | 0.0000 | undef | 0.0000 |
| Hepatic | 0.0046 | 0.0063 | 0.7324 | 1.3653 |
| Heart | 0.0010 | 0.0137 | 0.0738 | 13.5431 |
| Testicles | 0.0000 | 0.0118 | 0.0000 | undef |
| Lung | 0.0010 | 0.0055 | 0.1754 | 5.7011 |
| Stomach-esophagus | 0.0000 | 0.0064 | 0.0000 | undef |
| Muscle-skeleton | 0.0086 | 0.0148 | 0.5799 | 1.7246 |
| Kidney | 0.0112 | 0.0193 | 0.5803 | 1.7232 |
| Pancreas | 0.0017 | 0.0055 | 0.2992 | 3.3427 |
| Prostate | 0.0057 | 0.0117 | 0.4823 | 2.0732 |
| T lymphoma | 0.0000 | 0.0149 | 0.0000 | undef |
| Uterus | 0.0015 | 0.0092 | 0.1606 | 6.2251 |
| White blood cells | 0.0027 | 0.0000 | undef | 0.0000 |
| Hematopoietic | 0.0080 | | | |
| Penis | 0.0027 | | | |
| Seminal vesicle | 0.0070 | | | |
| Sensory organs | 0.0235 | | | |
| | FETUS | | | |
| Development | 0.0000 | | | |
| Gastrointestinal | 0.0028 | | | |
| Brain | 0.0000 | | | |
| Hematopoietic | 0.0039 | | | |
| Skin | 0.0000 | | | |
| Hepatic | 0.0000 | | | |
| Heart-blood vessels | 0.0000 | | | |
| Lung | 0.0145 | | | |
| Adrenal gland | 0.0000 | | | |
| Kidney | 0.0000 | | | |
| Placenta | 0.0061 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Breast_t | 0.0000 | | | |
| Large intestine_t | 0.0000 | | | |
| Ovary_n | 0.0000 | | | |
| Ovary_t | 0.0101 | | | |
| Endocrine tissue | 0.0000 | | | |
| Fetal | 0.0006 | | | |
| Gastrointestinal | 0.0000 | | | |
| Hematopoietic | 0.0000 | | | |
| Skin-muscle | 0.0000 | | | |
| Testicles_n | 0.0125 | | | |
| Testicles_t | 0.0000 | | | |
| Lungs_n | 0.0195 | | | |
| Lungs_t | 0.0000 | | | |
| Nerves | 0.0100 | | | |
| Kidney_t | 0.0000 | | | |
| Ovary_uterus | 0.0068 | | | |
| Prostate_n | 0.0121 | | | |
| Sensory organs | 0.0000 | | | |
| White blood cells | 0.0000 | | | |

Electronic Northern for Seq. ID: 152

| | | | | |
|---|---|---|---|---|
| | NORMAL | | | |
| B lymphoma | 0.0250 | 0.0136 | 1.8395 | 0.5436 |
| Bladder | 0.0078 | 0.0070 | 1.1063 | 0.9039 |
| Breast | 0.0097 | 0.0211 | 0.4592 | 2.1776 |
| Large intestine | 0.0192 | 0.0057 | 3.3639 | 0.2973 |
| Small intestine | 0.0055 | 0.0213 | 0.2577 | 3.8812 |
| Ovary | 0.0178 | 0.0143 | 1.2443 | 0.8036 |
| Endocrine tissue | 0.0048 | 0.0177 | 0.2716 | 3.6818 |
| Brain | 0.0191 | 0.0269 | 0.7092 | 1.4100 |
| Skin | 0.0073 | 0.0000 | undef | 0.0000 |
| Hepatic | 0.0046 | 0.0063 | 0.7324 | 1.3653 |
| Heart | 0.0233 | 0.0275 | 0.8491 | 1.1777 |
| Testicles | 0.0161 | 0.0000 | undef | 0.0000 |
| Lung | 0.0156 | 0.0129 | 1.2028 | 0.8314 |
| Stomach-esophagus | 0.0000 | 0.0192 | 0.0000 | undef |
| Muscle-skeleton | 0.0086 | 0.0074 | 1.1597 | 0.8623 |
| Kidney | 0.0090 | 0.0096 | 0.9285 | 1.0770 |
| Pancreas | 0.0083 | 0.0387 | 0.2137 | 4.6798 |
| Prostate | 0.0057 | 0.0091 | 0.6202 | 1.6125 |
| T lymphoma | 0.0076 | 0.0000 | undef | 0.0000 |
| Uterus | 0.0044 | 0.0000 | undef | 0.0000 |
| White blood cells | 0.0246 | 0.0000 | undef | 0.0000 |
| Hematopoietic | 0.0094 | | | |
| Penis | 0.0107 | | | |
| Seminal vesicle | 0.0000 | | | |
| Sensory organs | 0.0118 | | | |
| | FETUS | | | |
| Development | 0.1253 | | | |
| Gastrointestinal | 0.0305 | | | |
| Brain | 0.0938 | | | |
| Hematopoietic | 0.0275 | | | |
| Skin | 0.0000 | | | |
| Hepatic | 0.0000 | | | |
| Heart-blood vessels | 0.0142 | | | |
| Lung | 0.0325 | | | |
| Adrenal gland | 0.0254 | | | |
| Kidney | 0.0185 | | | |
| Placenta | 0.0242 | | | |
| Prostate | 0.0249 | | | |
| Sensory organs | 0.1255 | | | |
| | STANDARDIZED/ | | | |
| | SUBTRACTED | | | |
| | LIBRARIES | | | |
| Breast | 0.0816 | | | |
| Breast_t | 0.0000 | | | |
| Large intestine_t | 0.0000 | | | |
| Ovary_n | 0.0000 | | | |
| Ovary_t | 0.0354 | | | |
| Endocrine tissue | 0.0000 | | | |
| Fetal | 0.0741 | | | |
| Gastrointestinal | 0.0366 | | | |
| Hematopoietic | 0.0000 | | | |
| Skin-muscle | 0.1458 | | | |
| Testicles_n | 0.0000 | | | |
| Testicles_t | 0.0000 | | | |
| Lungs_n | 0.0098 | | | |
| Lungs_t | 0.0000 | | | |
| Nerves | 0.0191 | | | |
| Kidney_t | 0.0000 | | | |
| Ovary_uterus | 0.0248 | | | |
| Prostate_n | 0.0000 | | | |
| Sensory organs | 0.0155 | | | |
| White blood cells | 0.0000 | | | |

Electronic Northern for Seq. ID: 153

| | | | | |
|---|---|---|---|---|
| | NORMAL | | | |
| B lymphoma | 0.0000 | 0.0000 | undef | undef |
| Bladder | 0.0000 | 0.0000 | undef | undef |
| Breast | 0.0026 | 0.0141 | 0.1879 | 5.3230 |
| Large intestine | 0.0000 | 0.0057 | 0.0000 | undef |
| Small intestine | 0.0000 | 0.0000 | undef | undef |
| Ovary | 0.0000 | 0.0048 | 0.0000 | undef |
| Endocrine tissue | 0.0000 | 0.0000 | undef | undef |
| Brain | 0.0000 | 0.0010 | 0.0000 | undef |
| Skin | 0.0000 | 0.0000 | undef | undef |
| Hepatic | 0.0000 | 0.0063 | 0.0000 | undef |
| Heart | 0.0010 | 0.0000 | undef | 0.0000 |
| Testicles | 0.0000 | 0.0000 | undef | undef |
| Lung | 0.0039 | 0.0018 | 2.1049 | 0.4751 |
| Stomach-esophagus | 0.0145 | 0.0000 | undef | 0.0000 |
| Muscle-skeleton | 0.0000 | 0.0000 | undef | undef |
| Kidney | 0.0022 | 0.0000 | undef | 0.0000 |
| Pancreas | 0.0000 | 0.0000 | undef | undef |
| Prostate | 0.0009 | 0.0013 | 0.7235 | 1.3821 |
| T lymphoma | 0.0025 | 0.0000 | undef | 0.0000 |
| Uterus | 0.0015 | 0.0000 | undef | 0.0000 |
| White blood cells | 0.0000 | 0.0000 | undef | undef |
| Hematopoietic | 0.0000 | | | |
| Penis | 0.0054 | | | |
| Seminal vesicle | 0.0000 | | | |
| Sensory organs | 0.0000 | | | |
| | FETUS | | | |
| Development | 0.0000 | | | |
| Gastrointestinal | 0.0000 | | | |
| Brain | 0.0000 | | | |
| Hematopoietic | 0.0039 | | | |
| Skin | 0.0000 | | | |
| Hepatic | 0.0000 | | | |
| Heart-blood vessels | 0.0071 | | | |
| Lung | 0.0036 | | | |
| Adrenal gland | 0.0000 | | | |
| Kidney | 0.0000 | | | |
| Placenta | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Breast_t | 0.0000 | | | |
| Large intestine_t | 0.0000 | | | |
| Ovary_n | 0.0000 | | | |
| Ovary_t | 0.0051 | | | |
| Endocrine tissue | 0.0000 | | | |
| Fetal | 0.0000 | | | |
| Gastrointestinal | 0.0000 | | | |
| Hematopoietic | 0.0257 | | | |
| Skin-muscle | 0.0000 | | | |
| Testicles_n | 0.0000 | | | |
| Testicles_t | 0.0000 | | | |
| Lungs_n | 0.0000 | | | |
| Lungs_t | 0.0000 | | | |
| Nerves | 0.0010 | | | |
| Kidney_t | 0.0000 | | | |
| Ovary_uterus | 0.0023 | | | |
| Prostate_n | 0.0000 | | | |
| Sensory organs | 0.0000 | | | |
| White blood cells | 0.0000 | | | |

Electronic Northern for Seq. ID: 154

| | | | | |
|---|---|---|---|---|
| | NORMAL | | | |
| B lymphoma | 0.0000 | 0.0000 | undef | undef |
| Bladder | 0.0156 | 0.0000 | undef | 0.0000 |
| Breast | 0.0079 | 0.0112 | 0.7045 | 1.4195 |
| Large intestine | 0.0038 | 0.0000 | undef | 0.0000 |
| Small intestine | 0.0027 | 0.0000 | undef | 0.0000 |
| Ovary | 0.0119 | 0.0000 | undef | 0.0000 |
| Endocrine tissue | 0.0016 | 0.0000 | undef | 0.0000 |
| Brain | 0.0029 | 0.0060 | 0.4835 | 2.0680 |
| Skin | 0.0147 | 0.0000 | undef | 0.0000 |
| Hepatic | 0.0093 | 0.0000 | undef | 0.0000 |
| Heart | 0.0112 | 0.0000 | undef | 0.0000 |
| Testicles | 0.0000 | 0.0000 | undef | undef |
| Lung | 0.0097 | 0.0074 | 1.3155 | 0.7601 |
| Stomach-esophagus | 0.0000 | 0.0000 | undef | undef |
| Muscle-skeleton | 0.0051 | 0.0037 | 1.3917 | 0.7186 |
| Kidney | 0.0112 | 0.0048 | 2.3212 | 0.4308 |
| Pancreas | 0.0116 | 0.0000 | undef | 0.0000 |
| Prostate | 0.0057 | 0.0000 | undef | 0.0000 |
| T lymphoma | 0.0025 | 0.0075 | 0.3381 | 2.9576 |
| Uterus | 0.0044 | 0.0046 | 0.9638 | 1.0375 |
| White blood cells | 0.0048 | 0.0304 | 0.1578 | 6.3369 |
| Hematopoietic | 0.0027 | | | |
| Penis | 0.0080 | | | |
| Seminal vesicle | 0.0070 | | | |
| Sensory organs | 0.0235 | | | |
| | FETUS | | | |
| Development | 0.0000 | | | |
| Gastrointestinal | 0.0083 | | | |
| Brain | 0.0000 | | | |
| Hematopoietic | 0.0000 | | | |
| Skin | 0.0000 | | | |
| Hepatic | 0.0260 | | | |
| Heart-blood vessels | 0.0036 | | | |
| Lung | 0.0217 | | | |
| Adrenal gland | 0.0000 | | | |
| Kidney | 0.0000 | | | |
| Placenta | 0.0000 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Breast_t | 0.0000 | | | |
| Large intestine_t | 0.0000 | | | |
| Ovary_n | 0.0000 | | | |
| Ovary_t | 0.0000 | | | |
| Endocrine tissue | 0.0000 | | | |
| Fetal | 0.0185 | | | |
| Gastrointestinal | 0.0122 | | | |
| Hematopoietic | 0.0000 | | | |
| Skin-muscle | 0.0000 | | | |
| Testicles_n | 0.0042 | | | |
| Testicles_t | 0.0000 | | | |
| Lungs_n | 0.0000 | | | |
| Lungs_t | 0.0000 | | | |
| Nerves | 0.0050 | | | |
| Kidney_t | 0.0000 | | | |
| Ovary_uterus | 0.0000 | | | |
| Prostate_n | 0.0000 | | | |
| Sensory organs | 0.0000 | | | |
| White blood cells | 0.0000 | | | |

Electronic Northern for Seq. ID: 155

| | | | | |
|---|---|---|---|---|
| | NORMAL | | | |
| B lmphoma | 0.0075 | 0.0000 | undef | 0.0000 |
| Bladder | 0.0156 | 0.0047 | 3.3190 | 0.3013 |
| Breast | 0.0035 | 0.0197 | 0.1789 | 5.5892 |
| Large intestine | 0.0096 | 0.0085 | 1.1213 | 0.8918 |
| Small intestine | 0.0110 | 0.0000 | undef | 0.0000 |
| Ovary | 0.0059 | 0.0048 | 1.2443 | 0.8036 |
| Endocrine tissue | 0.0080 | 0.0124 | 0.6467 | 1.5464 |
| Brain | 0.0058 | 0.0010 | 5.8026 | 0.1723 |
| Skin | 0.0037 | 0.0000 | undef | 0.0000 |
| Hepatic | 0.0000 | 0.0063 | 0.0000 | undef |
| Heart | 0.0122 | 0.0000 | undef | 0.0000 |
| Testicles | 0.0201 | 0.0118 | 1.6964 | 0.5895 |
| Lung | 0.0010 | 0.0074 | 0.1316 | 7.6015 |
| Stomach-esophagus | 0.0217 | 0.0000 | undef | 0.0000 |
| Muscle-skeleton | 0.0051 | 0.0074 | 0.6958 | 1.4371 |
| Kidney | 0.0000 | 0.0000 | undef | undef |
| Pancreas | 0.0116 | 0.0000 | undef | 0.0000 |
| Prostate | 0.0047 | 0.0065 | 0.7235 | 1.3821 |
| T lymphoma | 0.0101 | 0.0075 | 1.3525 | 0.7394 |
| Uterus | 0.0044 | 0.0046 | 0.9638 | 1.0375 |
| White blood cells | 0.0048 | 0.0304 | 0.1578 | 6.3369 |
| Hematopoietic | 0.0040 | | | |
| Penis | 0.0134 | | | |
| Seminal vesicle | 0.0000 | | | |
| Sensory organs | 0.0000 | | | |
| | FETUS | | | |
| Development | 0.0278 | | | |
| Gastrointestinal | 0.0056 | | | |
| Brain | 0.0063 | | | |
| Hematopoietic | 0.0118 | | | |
| Skin | 0.0000 | | | |
| Hepatic | 0.0000 | | | |
| Heart-blood vessels | 0.0142 | | | |
| Lung | 0.0108 | | | |
| Adrenal gland | 0.0254 | | | |
| Kidney | 0.0124 | | | |
| Placenta | 0.0061 | | | |
| Prostate | 0.0499 | | | |
| Sensory organs | 0.0000 | | | |
| | STANDARDIZED/ | | | |
| | SUBTRACTED | | | |
| | LIBRARIES | | | |
| Breast | 0.0000 | | | |
| Breast_t | 0.0000 | | | |
| Large intestine_t | 0.0000 | | | |
| Ovary_n | 0.0000 | | | |
| Ovary_t | 0.0101 | | | |
| Endocrine tissue | 0.0000 | | | |
| Fetal | 0.0185 | | | |
| Gastrointestinal | 0.0122 | | | |
| Hematopoietic | 0.0000 | | | |
| Skin-muscle | 0.0194 | | | |
| Testicles_n | 0.0125 | | | |
| Testicles_t | 0.0000 | | | |
| Lungs_n | 0.0000 | | | |
| Lungs_t | 0.0000 | | | |
| Nerves | 0.0141 | | | |
| Kidney_t | 0.0000 | | | |
| Ovary_uterus | 0.0135 | | | |
| Prostate_n | 0.0121 | | | |
| Sensory organs | 0.0077 | | | |
| White blood cells | 0.0000 | | | |

Electronic Northern for Seq. ID: 156

| | | | | |
|---|---|---|---|---|
| | NORMAL | | | |
| B lymphoma | 0.0075 | 0.0000 | undef | 0.0000 |
| Bladder | 0.0000 | 0.0188 | 0.0000 | undef |
| Breast | 0.0026 | 0.0056 | 0.4697 | 2.1292 |
| Large intestine | 0.0038 | 0.0000 | undef | 0.0000 |
| Small intestine | 0.0055 | 0.0000 | undef | 0.0000 |
| Ovary | 0.0059 | 0.0048 | 1.2443 | 0.8036 |
| Endocrine tissue | 0.0032 | 0.0000 | undef | 0.0000 |
| Brain | 0.0017 | 0.0030 | 0.5803 | 1.7234 |
| Skin | 0.0000 | 0.0000 | undef | undef |
| Hepatic | 0.0046 | 0.0000 | undef | 0.0000 |
| Heart | 0.0051 | 0.0137 | 0.3692 | 2.7086 |
| Testicles | 0.0040 | 0.0000 | undef | 0.0000 |
| Lung | 0.0049 | 0.0129 | 0.3759 | 2.6605 |
| Stomach-esophagus | 0.0000 | 0.0000 | undef | undef |
| Muscle-skeleton | 0.0017 | 0.0037 | 0.4639 | 2.1557 |
| Kidney | 0.0000 | 0.0000 | undef | undef |
| Pancreas | 0.0017 | 0.0110 | 0.1496 | 6.6855 |
| Prostate | 0.0000 | 0.0013 | 0.0000 | undef |
| T lymphoma | 0.0051 | 0.0000 | undef | 0.0000 |
| Uterus | 0.0148 | 0.0046 | 3.2128 | 0.3113 |
| White blood cells | 0.0007 | 0.0000 | undef | 0.0000 |
| Hematopoietic | 0.0053 | | | |
| Penis | 0.0027 | | | |
| Seminal vesicle | 0.0000 | | | |
| Sensory organs | 0.0353 | | | |
| | FETUS | | | |
| Development | 0.0000 | | | |
| Gastrointestinal | 0.0000 | | | |
| Brain | 0.0000 | | | |
| Hematopoietic | 0.0118 | | | |
| Skin | 0.0000 | | | |
| Hepatic | 0.0000 | | | |
| Heart-blood vessels | 0.0000 | | | |
| Lung | 0.0072 | | | |
| Adrenal gland | 0.0000 | | | |
| Kidney | 0.0124 | | | |
| Placenta | 0.0242 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Breast_t | 0.0000 | | | |
| Large intestine_t | 0.0000 | | | |
| Ovary_n | 0.0000 | | | |
| Ovary_t | 0.0000 | | | |
| Endocrine tissue | 0.0245 | | | |
| Fetal | 0.0151 | | | |
| Gastrointestinal | 0.0000 | | | |
| Hematopoietic | 0.0000 | | | |
| Skin-muscle | 0.0259 | | | |
| Testicles_n | 0.0000 | | | |
| Testicles_t | 0.0000 | | | |
| Lungs_n | 0.0195 | | | |
| Lungs_t | 0.0000 | | | |
| Nerves | 0.0090 | | | |
| Kidney_t | 0.0000 | | | |
| Ovary_uterus | 0.0135 | | | |
| Prostate_n | 0.0000 | | | |
| Sensory organs | 0.0000 | | | |
| White blood cells | 0.0000 | | | |

Electronic Northern for Seq. ID: 157

| | | | | |
|---|---|---|---|---|
| | NORMAL | | | |
| B lymphoma | 0.0100 | 0.0136 | 0.7358 | 1.3590 |
| Bladder | 0.0000 | 0.0070 | 0.0000 | undef |
| Breast | 0.0106 | 0.0141 | 0.7515 | 1.3308 |
| Large intestine | 0.0057 | 0.0114 | 0.5046 | 1.9818 |
| Small intestine | 0.0027 | 0.0000 | undef | 0.0000 |
| Ovary | 0.0208 | 0.0215 | 0.9678 | 1.0333 |
| Endocrine tissue | 0.0193 | 0.0408 | 0.4724 | 2.1170 |
| Brain | 0.0087 | 0.0110 | 0.7913 | 1.2638 |
| Skin | 0.0037 | 0.0000 | undef | 0.0000 |
| Hepatic | 0.0000 | 0.0190 | 0.0000 | undef |
| Heart | 0.0101 | 0.0000 | undef | 0.0000 |
| Testicles | 0.0361 | 0.0118 | 3.0535 | 0.3275 |
| Lung | 0.0117 | 0.0185 | 0.6315 | 1.5836 |
| Stomach-esophagus | 0.0072 | 0.0000 | undef | 0.0000 |
| Muscle-skeleton | 0.0017 | 0.0369 | 0.0464 | 21.5570 |
| Kidney | 0.0201 | 0.0337 | 0.5969 | 1.6754 |
| Pancreas | 0.0149 | 0.0000 | undef | 0.0000 |
| Prostate | 0.0132 | 0.0247 | 0.5331 | 1.8758 |
| T lymphoma | 0.0177 | 0.0299 | 0.5917 | 1.6900 |
| Uterus | 0.0059 | 0.0046 | 1.2851 | 0.7781 |
| White blood cells | 0.0219 | 0.0000 | undef | 0.0000 |
| Hematopoietic | 0.0094 | | | |
| Penis | 0.0161 | | | |
| Seminal vesicle | 0.0070 | | | |
| Sensory organs | 0.0353 | | | |
| | FETUS | | | |
| Development | 0.0139 | | | |
| Gastrointestinal | 0.0167 | | | |
| Brain | 0.0000 | | | |
| Hematopoietic | 0.0039 | | | |
| Skin | 0.0000 | | | |
| Hepatic | 0.0000 | | | |
| Heart-blood vessels | 0.0142 | | | |
| Lung | 0.0036 | | | |
| Adrenal gland | 0.0254 | | | |
| Kidney | 0.0185 | | | |
| Placenta | 0.0061 | | | |
| Prostate | 0.0249 | | | |
| Sensory organs | 0.0126 | | | |
| | STANDARDIZED/ | | | |
| | SUBTRACTED | | | |
| | LIBRARIES | | | |
| Breast | 0.0000 | | | |
| Breast_t | 0.0000 | | | |
| Large intestine_t | 0.0000 | | | |
| Ovary_n | 0.0000 | | | |
| Ovary_t | 0.0203 | | | |
| Endocrine tissue | 0.0000 | | | |
| Fetal | 0.0209 | | | |
| Gastrointestinal | 0.0366 | | | |
| Hematopoietic | 0.0000 | | | |
| Skin-muscle | 0.0972 | | | |
| Testicles_n | 0.0251 | | | |
| Testicles_t | 0.0000 | | | |
| Lungs_n | 0.0098 | | | |
| Lungs_t | 0.0000 | | | |
| Nerves | 0.0151 | | | |
| Kidney_t | 0.0000 | | | |
| Ovary_uterus | 0.0450 | | | |
| Prostate_n | 0.0303 | | | |
| Sensory organs | 0.0310 | | | |
| White blood cells | 0.0000 | | | |

Electronic Northern for Seq. ID: 158

| | | | | |
|---|---|---|---|---|
| | NORMAL | | | |
| B lymphoma | 0.0075 | 0.0000 | undef | 0.0000 |
| Bladder | 0.0156 | 0.0070 | 2.2127 | 0.4519 |
| Breast | 0.0088 | 0.0155 | 0.5693 | 1.7566 |
| Large intestine | 0.0077 | 0.0057 | 1.3456 | 0.7432 |
| Small intestine | 0.0082 | 0.0000 | undef | 0.0000 |
| Ovary | 0.0000 | 0.0024 | 0.0000 | undef |
| Endocrine tissue | 0.0016 | 0.0000 | undef | 0.0000 |
| Brain | 0.0041 | 0.0209 | 0.1934 | 5.1701 |
| Skin | 0.0147 | 0.0000 | undef | 0.0000 |
| Hepatic | 0.0000 | 0.0190 | 0.0000 | undef |
| Heart | 0.0041 | 0.0000 | undef | 0.0000 |
| Testicles | 0.0120 | 0.0118 | 1.0178 | 0.9825 |
| Lung | 0.0146 | 0.0055 | 2.6311 | 0.3801 |
| Stomach-esophagus | 0.0000 | 0.0000 | undef | undef |
| Muscle-skeleton | 0.0051 | 0.0148 | 0.3479 | 2.8743 |
| Kidney | 0.0045 | 0.0096 | 0.4642 | 2.1540 |
| Pancreas | 0.0017 | 0.0055 | 0.2992 | 3.3427 |
| Prostate | 0.0047 | 0.0065 | 0.7235 | 1.3821 |
| T lymphoma | 0.0051 | 0.0000 | undef | 0.0000 |
| Uterus | 0.0163 | 0.0276 | 0.5890 | 1.6977 |
| White blood cells | 0.0000 | 0.0000 | undef | undef |
| Hematopoietic | 0.0053 | | | |
| Penis | 0.0107 | | | |
| Seminal vesicle | 0.0000 | | | |
| Sensory organs | 0.0118 | | | |
| | FETUS | | | |
| Development | 0.0000 | | | |
| Gastrointestinal | 0.0028 | | | |
| Brain | 0.0063 | | | |
| Hematopoietic | 0.0118 | | | |
| Skin | 0.0000 | | | |
| Hepatic | 0.0000 | | | |
| Heart-blood vessels | 0.0036 | | | |
| Lung | 0.0000 | | | |
| Adrenal gland | 0.0000 | | | |
| Kidney | 0.0124 | | | |
| Placenta | 0.0061 | | | |
| Prostate | 0.0499 | | | |
| Sensory organs | 0.0000 | | | |
| | STANDARDIZED/ | | | |
| | SUBTRACTED | | | |
| | LIBRARIES | | | |
| Breast | 0.0000 | | | |
| Breast_t | 0.0000 | | | |
| Large intestine_t | 0.0000 | | | |
| Ovary_n | 0.0000 | | | |
| Ovary_t | 0.0000 | | | |
| Endocrine tissue | 0.0000 | | | |
| Fetal | 0.0041 | | | |
| Gastrointestinal | 0.0000 | | | |
| Hematopoietic | 0.0257 | | | |
| Skin-muscle | 0.0194 | | | |
| Testicles_n | 0.0000 | | | |
| Testicles_t | 0.0000 | | | |
| Lungs_n | 0.0098 | | | |
| Lungs_t | 0.0000 | | | |
| Nerves | 0.0060 | | | |
| Kidney_t | 0.0000 | | | |
| Ovary_uterus | 0.0068 | | | |
| Prostate_n | 0.0000 | | | |
| Sensory organs | 0.0000 | | | |
| White blood cells | 0.0000 | | | |

Electronic Northern for Seq. ID: 159

| | | | | |
|---|---|---|---|---|
| | NORMAL | | | |
| B lymphoma | 0.0050 | 0.0407 | 0.1226 | 8.1542 |
| Bladder | 0.0000 | 0.0047 | 0.0000 | undef |
| Breast | 0.0097 | 0.0098 | 0.9840 | 1.0162 |
| Large intestine | 0.0077 | 0.0171 | 0.4485 | 2.2295 |
| Small intestine | 0.0055 | 0.0107 | 0.5153 | 1.9406 |
| Ovary | 0.0089 | 0.0072 | 1.2443 | 0.8036 |
| Endocrine tissue | 0.0064 | 0.0106 | 0.6036 | 1.6568 |
| Brain | 0.0075 | 0.0090 | 0.8382 | 1.1931 |
| Skin | 0.0000 | 0.0000 | undef | undef |
| Hepatic | 0.0093 | 0.0000 | undef | 0.0000 |
| Heart | 0.0000 | 0.0000 | undef | undef |
| Testicles | 0.0080 | 0.0059 | 1.3571 | 0.7369 |
| Lung | 0.0068 | 0.0055 | 1.2278 | 0.8144 |
| Stomach-esophagus | 0.0000 | 0.0064 | 0.0000 | undef |
| Muscle-skeleton | 0.0034 | 0.0000 | undef | 0.0000 |
| Kidney | 0.0045 | 0.0096 | 0.4642 | 2.1540 |
| Pancreas | 0.0017 | 0.0000 | undef | 0.0000 |
| Prostate | 0.0066 | 0.0013 | 5.0646 | 0.1974 |
| T lymphoma | 0.0101 | 0.0075 | 1.3525 | 0.7394 |
| Uterus | 0.0044 | 0.0230 | 0.1928 | 5.1876 |
| White blood cells | 0.0041 | 0.0000 | undef | 0.0000 |
| Hematopoietic | 0.0147 | | | |
| Penis | 0.0027 | | | |
| Seminal vesicle | 0.0000 | | | |
| Sensory organs | 0.0000 | | | |
| | FETUS | | | |
| Development | 0.0000 | | | |
| Gastrointestinal | 0.0028 | | | |
| Brain | 0.0063 | | | |
| Hematopoietic | 0.0079 | | | |
| Skin | 0.0000 | | | |
| Hepatic | 0.0000 | | | |
| Heart-blood vessels | 0.0036 | | | |
| Lung | 0.0072 | | | |
| Adrenal gland | 0.0254 | | | |
| Kidney | 0.0247 | | | |
| Placenta | 0.0121 | | | |
| Prostate | 0.0000 | | | |
| Sensory organs | 0.0126 | | | |
| | STANDARDIZED/ | | | |
| | SUBTRACTED | | | |
| | LIBRARIES | | | |
| Breast | 0.0000 | | | |
| Breast_t | 0.0000 | | | |
| Large intestine_t | 0.0000 | | | |
| Ovary_n | 0.0000 | | | |
| Ovary_t | 0.0203 | | | |
| Endocrine tissue | 0.0000 | | | |
| Fetal | 0.0087 | | | |
| Gastrointestinal | 0.0244 | | | |
| Hematopoietic | 0.0257 | | | |
| Skin-muscle | 0.0097 | | | |
| Testicles_n | 0.0209 | | | |
| Testicles_t | 0.0000 | | | |
| Lungs_n | 0.0000 | | | |
| Lungs_t | 0.0000 | | | |
| Nerves | 0.0030 | | | |
| Kidney_t | 0.0000 | | | |
| Ovary_uterus | 0.0090 | | | |
| Prostate_n | 0.0000 | | | |
| Sensory organs | 0.0774 | | | |
| White blood cells | 0.0000 | | | |

Electronic Northern for Seq. ID: 160

| | | | | |
|---|---|---|---|---|
| | NORMAL | | | |
| B lymphoma | 0.0000 | 0.0000 | undef | undef |
| Bladder | 0.0078 | 0.0023 | 3.3190 | 0.3013 |
| Breast | 0.0009 | 0.0084 | 0.1044 | 9.5814 |
| Large intestine | 0.0057 | 0.0000 | undef | 0.0000 |
| Small intestine | 0.0027 | 0.0107 | 0.2577 | 3.8812 |
| Ovary | 0.0030 | 0.0048 | 0.6222 | 1.6073 |
| Endocrine tissue | 0.0016 | 0.0018 | 0.9054 | 1.1045 |
| Brain | 0.0041 | 0.0040 | 1.0155 | 0.9848 |
| Skin | 0.0000 | 0.0000 | undef | undef |
| Hepatic | 0.0186 | 0.0000 | undef | 0.0000 |
| Heart | 0.0010 | 0.0000 | undef | 0.0000 |
| Testicles | 0.0000 | 0.0000 | undef | undef |
| Lung | 0.0019 | 0.0000 | undef | 0.0000 |
| Stomach-esophagus | 0.0000 | 0.0000 | undef | undef |
| Muscle-skeleton | 0.0017 | 0.0037 | 0.4639 | 2.1557 |
| Kidney | 0.0045 | 0.0000 | undef | 0.0000 |
| Pancreas | 0.0017 | 0.0055 | 0.2992 | 3.3427 |
| Prostate | 0.0009 | 0.0000 | undef | 0.0000 |
| T lymphoma | 0.0076 | 0.0075 | 1.0143 | 0.9859 |
| Uterus | 0.0030 | 0.0000 | undef | 0.0000 |
| White blood cells | 0.0041 | 0.0000 | undef | 0.0000 |
| Hematopoietic | 0.0040 | | | |
| Penis | 0.0080 | | | |
| Seminal vesicle | 0.0070 | | | |
| Sensory organs | 0.0000 | | | |
| | FETUS | | | |
| Development | 0.0000 | | | |
| Gastrointestinal | 0.0111 | | | |
| Brain | 0.0125 | | | |
| Hematopoietic | 0.0039 | | | |
| Skin | 0.0000 | | | |
| Hepatic | 0.0000 | | | |
| Heart-blood vessels | 0.0071 | | | |
| Lung | 0.0000 | | | |
| Adrenal gland | 0.0000 | | | |
| Kidney | 0.0062 | | | |
| Placenta | 0.0121 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Breast_t | 0.0000 | | | |
| Large intestine_t | 0.0000 | | | |
| Ovary_n | 0.0000 | | | |
| Ovary_t | 0.0101 | | | |
| Endocrine tissue | 0.0490 | | | |
| Fetal | 0.0058 | | | |
| Gastrointestinal | 0.0000 | | | |
| Hematopoietic | 0.0000 | | | |
| Skin-muscle | 0.0065 | | | |
| Testicles_n | 0.0000 | | | |
| Testicles_t | 0.0000 | | | |
| Lungs_n | 0.0000 | | | |
| Lungs_t | 0.0000 | | | |
| Nerves | 0.0020 | | | |
| Kidney_t | 0.0000 | | | |
| Ovary_uterus | 0.0045 | | | |
| Prostate_n | 0.0000 | | | |
| Sensory organs | 0.0000 | | | |
| White blood cells | 0.0000 | | | |

Electronic Northern for Seq. ID: 161

| | | | | |
|---|---|---|---|---|
| | NORMAL | | | |
| B lymphoma | 0.0050 | 0.0000 | undef | 0.0000 |
| Bladder | 0.0078 | 0.0047 | 1.6595 | 0.6026 |
| Breast | 0.0009 | 0.0084 | 0.1044 | 9.5814 |
| Large intestine | 0.0000 | 0.0000 | undef | undef |
| Small intestine | 0.0000 | 0.0107 | 0.0000 | undef |
| Ovary | 0.0000 | 0.0048 | 0.0000 | undef |
| Endocrine tissue | 0.0064 | 0.0106 | 0.6036 | 1.6568 |
| Brain | 0.0058 | 0.0050 | 1.1605 | 0.8617 |
| Skin | 0.0000 | 0.0000 | undef | undef |
| Hepatic | 0.0000 | 0.0190 | 0.0000 | undef |
| Heart | 0.0041 | 0.0000 | undef | 0.0000 |
| Testicles | 0.0040 | 0.0118 | 0.3393 | 2.9475 |
| Lung | 0.0039 | 0.0018 | 2.1049 | 0.4751 |
| Stomach-esophagus | 0.0000 | 0.0000 | undef | undef |
| Muscle-skeleton | 0.0000 | 0.0000 | undef | undef |
| Kidney | 0.0022 | 0.0048 | 0.4642 | 2.1540 |
| Pancreas | 0.0050 | 0.0055 | 0.8975 | 1.1142 |
| Prostate | 0.0848 | 0.0651 | 1.3023 | 0.7679 |
| T lymphoma | 0.0076 | 0.0075 | 1.0143 | 0.9859 |
| Uterus | 0.0059 | 0.0046 | 1.2851 | 0.7781 |
| White blood cells | 0.0041 | 0.0000 | undef | 0.0000 |
| Hematopoietic | 0.0027 | | | |
| Penis | 0.0027 | | | |
| Seminal vesicle | 0.0000 | | | |
| Sensory organs | 0.0000 | | | |
| | FETUS | | | |
| Development | 0.0000 | | | |
| Gastrointestinal | 0.0056 | | | |
| Brain | 0.0000 | | | |
| Hematopoietic | 0.0039 | | | |
| Skin | 0.0000 | | | |
| Hepatic | 0.0000 | | | |
| Heart-blood vessels | 0.0000 | | | |
| Lung | 0.0036 | | | |
| Adrenal gland | 0.0507 | | | |
| Kidney | 0.0000 | | | |
| Placenta | 0.0121 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Breast_t | 0.0000 | | | |
| Large intestine_t | 0.0000 | | | |
| Ovary_n | 0.0000 | | | |
| Ovary_t | 0.0000 | | | |
| Endocrine tissue | 0.0000 | | | |
| Fetal | 0.0104 | | | |
| Gastrointestinal | 0.0000 | | | |
| Hematopoietic | 0.0000 | | | |
| Skin-muscle | 0.0097 | | | |
| Testicles_n | 0.0084 | | | |
| Testicles_t | 0.0000 | | | |
| Lungs_n | 0.0000 | | | |
| Lungs_t | 0.0000 | | | |
| Nerves | 0.0050 | | | |
| Kidney_t | 0.0000 | | | |
| Ovary_uterus | 0.0000 | | | |
| Prostate_n | 0.0546 | | | |
| Sensory organs | 0.0000 | | | |
| White blood cells | 0.0000 | | | |

Electronic Northern for SEQ. ID NO.: 201

| | | | | |
|---|---|---|---|---|
| | NORMAL | | | |
| Bladder | 0.0000 | 0.0077 | 0.0000 | undef |
| Breast | 0.0027 | 0.0305 | 0.0874 | 11.4458 |
| Ovary | 0.0030 | 0.0833 | 0.0365 | 27.3828 |
| Endocrine tissue | 0.0018 | 0.0109 | 0.1674 | 5.9721 |
| Gastrointestinal | 0.0000 | 0.0048 | 0.0000 | undef |
| Brain | 0.0576 | 0.0548 | 1.0528 | 0.9499 |
| Hematopoietic | 0.0042 | 0.0000 | undef | 0.0000 |
| Skin | 0.0099 | 0.1693 | 0.0587 | 17.0262 |
| Hepatic | 0.0446 | 0.1812 | 0.2459 | 4.0660 |
| Heart | 0.0138 | 0.0137 | 1.0020 | 0.9980 |
| Testicles | 0.0000 | 0.1052 | 0.0000 | undef |
| Lungs | 0.0087 | 0.0851 | 0.1024 | 9.7640 |
| Stomach-esophagus | 0.0000 | 0.0230 | 0.0000 | undef |
| Muscle-skeleton | 0.0274 | 0.2820 | 0.0972 | 10.2887 |
| Kidneys | 0.3448 | 0.4245 | 0.8123 | 1.2311 |
| Pancreas | 0.1268 | 0.0387 | 3.2814 | 0.3047 |
| Penis | 0.0030 | 0.0000 | undef | 0.0000 |
| Prostate | 0.0000 | 0.0021 | 0.0000 | undef |
| Uterus | 0.0033 | 0.0427 | 0.0774 | 12.9263 |
| Breast hyperplasia | 0.0073 | | | |
| Small intestine | 0.0000 | | | |
| Prostatic hyperplasia | 0.0059 | | | |
| Seminal vesicles | 0.0000 | | | |
| Sensory organs | 0.0118 | | | |
| White blood cells | 0.1958 | | | |
| | FETUS | | | |
| Development | 0.0922 | | | |
| Gastrointestinal | 0.0247 | | | |
| Brain | 0.0000 | | | |
| Hematopoietic | 0.0079 | | | |
| Heart-blood vessels | 0.0123 | | | |
| Lungs | 0.0037 | | | |
| Kidneys | 0.0062 | | | |
| Prostate | 0.0000 | | | |
| Sensory organs | 0.1815 | | | |
| | STANDARDIZED/ | | | |
| | SUBTRACTED | | | |
| | LIBRARIES | | | |
| Breast | 0.0000 | | | |
| Ovary-uterus | 0.0000 | | | |
| Endocrine tissue | 0.0000 | | | |
| Fetal | 0.0134 | | | |
| Gastrointestinal | 0.0000 | | | |
| Hematopoietic | 0.0000 | | | |
| Skin-muscle | 0.0032 | | | |
| Testicles | 0.0000 | | | |
| Lungs | 0.0164 | | | |
| Nerves | 0.0231 | | | |
| Prostate | 0.0064 | | | |
| Sensory organs | 0.0310 | | | |

Electronic Northern for SEQ. ID NO.: 202

| | | | | |
|---|---|---|---|---|
| | NORMAL | | | |
| Bladder | 0.0000 | 0.0792 | 0.0000 | undef |
| Breast | 0.0040 | 0.0261 | 0.1529 | 6.5404 |
| Ovary | 0.0091 | 0.0156 | 0.5843 | 1.7114 |
| Endocrine tissue | 0.0036 | 0.0027 | 1.3396 | 0.7465 |
| Gastrointestinal | 0.0174 | 0.0143 | 1.2214 | 0.8187 |
| Brain | 0.0000 | 0.0044 | 0.0000 | undef |
| Hematopoietic | 0.0056 | 0.0000 | undef | 0.0000 |
| Skin | 0.0199 | 0.0000 | undef | 0.0000 |
| Hepatic | 0.0199 | 0.0647 | 0.3061 | 3.2673 |
| Heart | 0.0011 | 0.0000 | undef | 0.0000 |
| Testicles | 0.0061 | 0.0000 | undef | 0.0000 |
| Lungs | 0.0224 | 0.0118 | 1.8962 | 0.5274 |
| Stomach-esophagus | 0.0193 | 0.0000 | undef | 0.0000 |
| Muscle-skeleton | 0.0034 | 0.0000 | undef | 0.0000 |
| Kidneys | 0.0178 | 0.0068 | 2.6050 | 0.3839 |
| Pancreas | 0.0019 | 0.0331 | 0.0571 | 17.5010 |
| Penis | 0.0060 | 0.0800 | 0.0749 | 13.3560 |
| Prostate | 0.0095 | 0.0064 | 1.4915 | 0.6705 |
| Uterus | 0.0017 | 0.0000 | undef | 0.0000 |
| Breast hyperplasia | 0.0000 | | | |
| Small intestine | 0.0031 | | | |
| Prostatic hyperplasia | 0.0119 | | | |
| Seminal vesicles | 0.0000 | | | |
| Sensory organs | 0.0235 | | | |
| White blood cells | 0.0000 | | | |
| | FETUS | | | |
| Development | 0.0000 | | | |
| Gastrointestinal | 0.0092 | | | |
| Brain | 0.0063 | | | |
| Hematopoietic | 0.0000 | | | |
| Heart-blood vessels | 0.0000 | | | |
| Lungs | 0.0259 | | | |
| Kidneys | 0.0062 | | | |
| Prostate | 0.0000 | | | |
| Sensory organs | 0.0000 | | | |
| | STANDARDIZED/ | | | |
| | SUBTRACTED | | | |
| | LIBRARIES | | | |
| Breast | 0.0000 | | | |
| Ovary-uterus | 0.0068 | | | |
| Endocrine tissue | 0.0000 | | | |
| Fetal | 0.0274 | | | |
| Gastrointestinal | 0.0122 | | | |
| Hematopoietic | 0.0000 | | | |
| Skin-muscle | 0.0000 | | | |
| Testicles | 0.0000 | | | |
| Lungs | 0.0410 | | | |
| Nerves | 0.0000 | | | |
| Prostate | 0.0064 | | | |
| Sensory organs | 0.0000 | | | |

2.2. Fisher Test

In order to decide whether a partial sequence S of a gene occurs significantly more often or less often in a library for normal tissue than in a library for degenerated tissue, Fisher's exact test, a standard statistical process, is carried out (Hays, W. L., (1991) Statistics, Harcourt Brace College Publishers, Fort Worth).

The null hypothesis reads: The two libraries cannot be distinguished with respect to the frequency of sequences homologous to S. If the null hypothesis can be rejected with high enough certainty, the gene belonging to S is accepted as an advantageous candidate for a cancer gene, and in the next step an attempt is made to achieve lengthening of its sequence.

### EXAMPLE 3

Automatic Lengthening of the Partial Sequence

Automatic lengthening of partial sequence S is completed in three steps:

1. Determination of all sequences homologous to S from the total set of available sequences using BLAST.

2. Assembling these sequences by means of the standard program GAP4 (Bonfield, J. K.; Smith, K. F. and Staden, R. (1995), Nucleic Acids Research 23 4992-4999) (contig formation).

3. Computation of a consensus sequence C from the assembled sequences.

The consensus sequence C will generally be longer than initial sequence S. Its electronic Northern Blot will accordingly deviate from that for S. A repeated Fisher test decides whether the alternative hypothesis of deviation from a uniform expression in the two libraries can be maintained. If this is the case, an attempt is made to lengthen C in the same way as S. This iteration is continued with consensus sequences Cᵢ (i: iteration index) obtained in each case until the alternative hypothesis is rejected (if H₀ Exit; truncation criterion I) or until automatic lengthening is no longer possible (while Cᵢ>Cᵢ₋₁; truncation criterion II).

In the case of truncation criterion II, with the consensus sequence present after the last iteration, a complete or roughly complete sequence of a gene which can be related to cancer with high statistical certainty is acquired.

Analogously to the above-described examples, it was possible to find the nucleic acid sequences described in Table I from breast tumor tissue.

Furthermore, for the individual nucleic acid sequences, it was possible to determine the peptide sequences (ORFs) that are listed in Table II, in which no peptide can be assigned to a few nucleic acid sequences and more than one peptide can be assigned to some nucleic acid sequences. As already mentioned above, both the determined nucleic acid sequences and the peptide sequences assigned to the nucleic acid sequences are the subject of this invention.

### EXAMPLE 4

Mapping of Nucleic Acid Sequences on the Human Genome

Human genes were mapped using the Stanford G3 Hybrid Panel (Stewart et al., 1997), which is marketed by Research Genetics, Huntsville, Ala. This panel consists of 83 different genomic DNAs of human-hamster hybrid cell lines and allows resolution of 500 kilobases. The hybrid cell lines were obtained by fusion of irradiated diploid human cells with cells of the Chinese hamster. The retention pattern of the human chromosome fragments is determined by means of gene-specific primers in a polymerase chain reaction and is analyzed using software available from the Stanford RH server (http://www.stanford.edu/RH/rhserver_form2. html). This program determines the STS marker that is nearest to the desired gene. The corresponding cytogenetic band was determined using the “Mapview” program of the Genome Database (GDB), (http://gdbwww.dkfz-heidelberg.de).

In addition to mapping of genes on the human chromosome set by various experimental methods, it is possible to determine the location of genes on this by biocomputer methods. To do this, the known program e-PCR was used (Schuler G D (1998) Electronic PCR: Bridging the gap between genome mapping and genome sequencing. Trends Biotechnol 16: 456-459, Schuler G D (1997). Sequence mapping by electronic PCR. Genome Res. 7: 541-550). The database used here no longer corresponds to the one cited in the literature, but is a further development which includes data from the public database RHdb (http://www.ebi.ac.uk/RHdb/-index.html). Analogously to the mapping by the hybrid panels, the results were evaluated with the above-mentioned software and the software of the Whitehead Institute (http://carbon.wi.mit.edu:8000/cgi-bin/contig/rhmapper.pl).

### EXAMPLE 5

Obtaining Genomic DNA Sequences (BAC Clones)

The genomic sequences that correspond to the differential cDNAs were isolated from commercial BAC libraries. BAC libraries of the Genome Systems Company, St. Louis, Mo., which were produced from human lymphocytes, were used (http://www.genomesystems.com) and those of the Research Genetics Company, which were described as follows, were used: Shizuya, H.; B. Birren, U-J. Kim, V. Mancino, T. Slepak, Y. Tachiiri, M. Simon (1992) Proc. Natl. Acad. Sci., USA 89: 8794-8797; http://www.tree.caltech.edu/. From these libraries, the BAC clones with the procedure of “down-to-the-well” were isolated. In this procedure, a library consisting of BAC clones (the library covers roughly 3× the human genome) is combined in various groups. This takes place in such a way that after the execution of a gene-specific PCR in the various pools, a definite clone assignment is possible. This address together with the name of the library which is being used unequivocally fixes the clones and thus the DNA sequence of these clones.

The following examples explain the successful isolation of the genomic BAC clones without limiting them thereto.

The libraries of genome systems used were CITB B and CITB C. Clones from the library of genome systems are underlined. Breast Tumor

| Seq. ID | | | | | |
|---|---|---|---|---|---|
| No. | Identified BACs | | | | |
| 3 | 431/F/22 | 461/J/18 | 276/D/4 | 360/G/5 | 276/D/5 |
| 7 | 241/D/11 | | | | |
| 9 | 13/M/23 | 102/H/20 | 210/0/17 | 278/B/10 | 278/B/20 |
| 10 | 319/P/11 | 492/J/15 | | | |
| 23 | 565/E/8 | | | | |
| 25 | 38/D/4 | 60/B/17 | 70/K/14 | | |
| 39 | 425/C/18 | | | | |
| 42 | 221/L/9 | 407/M/9 | | | |
| 43 | 233/F/11 | 411/C/6 | 411/C/8 | 461/C/20 | |
| 45 | 557/D/15 | | | | |
| 58 | 222/C/8 | 431/0/16 | | | |

TABLE I

| | |
|---|---|
| Col. 1 | Seq ID No. |
| Col. 2 | Expression |
| Col. 3 | Function |
| Col. 4 | Modules |
| Col. 5 | Length of the applied sequence (bp) |
| Col. 6 | Chromosomal localization |
| Col. 7 | Marker |

| Seq | | |
|---|---|---|
| ID No. | Expression | Function |
| 2 | Overexpressed in | A 17-kDA interferon-induced gene— |
| | the breast tumor | nothing is published on its function |
| 3 | Overexpressed in | Presumably the human ortholog of the |
| | the breast tumor | 5E5-antigen of rats, in this connection |
| | | it is probably a transcription factor. |
| 4 | Overexpressed in | The human “alpha-2-macroglobulin |
| | the breast tumor | receptor-associated protein” belongs to |
| | | a “proteinase-scavenging system” that |
| | | recovers proteinase activity. |
| 5 | Overexpressed in | The human “macrophage migration |
| | the breast tumor | inhibition factor related protein 14 |
| | | (MRP-14)” probably plays a role in |
| | | immunomodulation. |
| 6 | Overexpressed in | The human lamin B2 (LAMB2) forms, |
| | the breast tumor | i.a., the nuclear lamina, which lies |
| | | below the inside cell membrane of the |
| | | nucleus. It plays an important role in |
| | | the regulation of the nuclear structure |
| | | during the cell cycle and the |
| | | transcription. |
| 7 | Overexpressed in | Presumably the regulatory subunit of |
| | the breast tumor | an RNA-binding protein,; nothing was |
| | | published on its function. |
| 8 | Overexpressed in | The human antioxidant enzyme AOE37- |
| | the breast tumor | 2, which presumably represents a |
| | | peroxidase (peroxiredoxin family). |
| | | The latter protect the cells from |
| | | oxidative processes. |
| 9 | Overexpressed in | Unknown |
| | the breast tumor | |
| 11 | Overexpressed in | The human “macrophage capping |
| | the breast tumor | protein,” recently named “CapG,” |
| | | regulates the cell mobility via the actins. |
| 12 | Overexpressed in | An estrogen-inducible gene (LIV-1), |
| | the breast tumor | whose function is not yet understood. |
| 13 | Overexpressed in | The human “integrin-linked kinase |
| | the breast tumor | (ILK)” controls the assembly of the |
| | | fibrinectin matrix and inhibits the |
| | | synthesis of E-cadherin. ILK- |
| | | coverexpressing cells produce tumors |
| | | in nude mice. |
| 14 | Overexpressed in | Unknown, human HISTONE H2B2 |
| | the breast tumor | |
| 15 | Overexpressed in | Presumably the human ortholog of |
| | the breast tumor | the enhancer of the Drosophila |
| | | “rudimentary” gene (“human enhancer |
| | | of rudimentary homolog”); it possibly |
| | | plays a role in the pyrimidine |
| | | metabolism. |
| 16 | Overexpressed in | The human Tim23 is localized in the |
| | the breast tumor | protein translocase complex of the |
| | | inside mitochondrial membrane. |
| 17 | Overexpressed in | Presumably a new Ca2+-binding |
| | the breast tumor | protein. |
| 18 | Overexpressed in | A new human gene with similarity to |
| | the breast tumor | the mouse “synaptosomal associated |
| | | protein.” |
| 19 | Overexpressed in | The human ITF (“intestinal trefoil |
| | the breast tumor | factor“). |
| 20 | Overexpressed in | The human RNA polymerase II |
| | the breast tumor | transcription factor. |
| 21 | Overexpressed in | Unknown |
| | the breast tumor | |
| 22 | Overexpressed in | The human JAK1 tyrosine kinase. |
| | the breast tumor | |
| 23 | Overexpressed in | Unknown |
| | the breast tumor | |
| 24 | Overexpressed in | Unknown |
| | the breast tumor | |
| 25 | Overexpressed in | Unknown |
| | the breast tumor | |
| 27 | Overexpressed in | Unknown |
| | the breast tumor | |
| 29 | Overexpressed in | The human ribonuclease 6-precursor |
| | the breast tumor | molecule. |
| 30 | Overexpressed in | The human 80K-L protein (also |
| | the breast tumor | MARCKS), a substrate of the protein |
| | | kinase C. MARCKS binds calmodulin, |
| | | actin and synapsin. |
| 31 | Overexpressed in | The human BCL-X, an apoptosis |
| | the breast tumor | regulator. |
| 33 | Overexpressed in | The human benzodiazepine receptor. |
| | the breast tumor | |
| 35 | Overexpressed in | The human ubiquinone oxidoreductase. |
| | the breast tumor | |
| 36 | Overexpressed in | A new ATPase, which belongs to the |
| | the breast tumor | kinesin family. |
| 37 | Overexpressed in | The monocyte/macrophage “Ig- |
| | the breast tumor | related receptor MIR-7.” |
| 38 | Overexpressed in | Unknown |
| | the breast tumor | |
| 39 | Overexpressed in | A new putative serine/threonine |
| | the breast tumor | kinase. |
| 40 | Overexpressed in | The putative copper-receiving |
| | the breast tumor | protein hCTR2. |
| 41 | Overexpressed in | The human alpha galactosidase A. |
| | the breast tumor | |
| 42 | Overexpressed in | Unknown |
| | the breast tumor | |
| 43 | Overexpressed in | This new sequence has a certain |
| | the breast tumor | homology to the benzoate-coenzyme A |
| | | ligase. |
| 44 | Overexpressed in | 7TM protein |
| | the breast tumor | |
| 45 | Overexpressed in | The human phosphatase 2A B56 |
| | the breast tumor | (PP2A). |
| 46 | Overexpressed in | HUMAN homolog to a mouse co- |
| | the breast tumor | chaperonin; Hs. 24930; homo sapiens |
| | | cofactor A protein mRNA |
| 47 | Overexpressed in | Homolog to an imprinted gene of |
| | the breast tumor | chromosome 11; Hs. 110222 |
| 48 | Overexpressed in | Presumably the human ortholog of |
| | the breast tumor | an ATP-dependent RNA-helicase. |
| 49 | Overexpressed in | Coupling factor F6 is a component of |
| | the breast tumor | the mitochondrial ATP-synthase, which |
| | | is necessary for the interaction of |
| | | catalytic and proton-transferring |
| | | segments; Hs. 73851 |
| 50 | Overexpressed in | Unknown |
| | the breast tumor | |
| 51 | Overexpressed in | New human ATPase |
| | the breast tumor | |
| 52 | Overexpressed in | Unknown; Hs. 10927 |
| | the breast tumor | |
| 53 | Overexpressed in | Homolog to the NAG-2 gene; Hs. |
| | the breast tumor | 26518 |
| 54 | Overexpressed in | Arginine methyltransferase; Hs. |
| | the breast tumor | 20521 |
| 55 | Overexpressed in | Unknown; Hs. 5241 |
| | the breast tumor | |
| 56 | Overexpressed in | Stromelysine |
| | the breast tumor | |
| 57 | Overexpressed in | Human GTP-binding protein |
| | the breast tumor | |
| 58 | Overexpressed in | Homolog to prostate-binding protein, |
| | the breast tumor | subunit C-1 |
| 59 | Overexpressed in | Unknown |
| | the breast tumor | |
| 61 | Overexpressed in | Glucose-6-phosphate dehydrogenase |
| | the breast tumor | homolog |
| 62 | Overexpressed in | Unknown |
| | the breast tumor | |
| 63 | Overexpressed in | Unknown |
| | the breast tumor | |
| 64 | Overexpressed in | Ets transcription factor |
| | the breast tumor | |
| 65 | Overexpressed in | IL 13 receptor alpha-1 chain |
| | the breast tumor | |
| 66 | Overexpressed in | Inhibition of cell division and the |
| | the breast tumor | macrophage activity. Protein-kinase |
| | | inhibitor |
| 67 | Overexpressed in | Possibly a dehydrogenase |
| | the breast tumor | |
| 149 | Overexpressed in | Lengthening to Seq. ID No. 4 |
| | the breast tumor | |
| 150 | Overexpressed in | Lengthening to Seq. ID No. 9 |
| | the breast tumor | |
| 151 | Overexpressed in | Lengthening to Seq. ID No. 25 |
| | the breast tumor | |
| 152 | Overexpressed in | Lengthening to Seq. ID No. 27 |
| | the breast tumor | |
| 153 | Overexpressed in | Lengthening to Seq. ID No. 44 |
| | the breast tumor | |
| 154 | Overexpressed in | Lengthening to Seq. ID No. 45 |
| | the breast tumor | |
| 155 | Overexpressed in | Lengthening to Seq. ID No. 48 |
| | the breast tumor | |
| 156 | Overexpressed in | Lengthening to Seq. ID No. 50 |
| | the breast tumor | |
| 157 | Overexpressed in | Lengthening to Seq. ID No. 51 |
| | the breast tumor | |
| 158 | Overexpressed in | Lengthening to Seq. ID No. 53 |
| | the breast tumor | |
| 159 | Overexpressed in | Lengthening to Seq. ID No. 62 |
| | the breast tumor | |
| 160 | Overexpressed in | Lengthening to Seq. ID No. 63 |
| | the breast tumor | |
| 161 | Overexpressed in | Lengthening to Seq. ID No. 67 |
| | the breast tumor | |
| 201 | Overexpressed in | The human osteopontin. |
| | the breast tumor | |
| 202 | Overexpressed in | Proteoglycan |
| | the breast tumor | |

[Key:]
unbekannt = unknown

TABLE II

| DNA Sequence | Peptide Sequence | | | | |
|---|---|---|---|---|---|
| Seq. ID. No. | (ORF's) Seq. ID. No. | | | | |
| 3 | 71 | | | | |
| 9 | 72 | 73 | 74 | 75 | 76 |
| 14 | 77 | | | | |
| 16 | 78 | | | | |
| 17 | 79 | | | | |
| 18 | 81 | | | | |
| 19 | 82 | | | | |
| 20 | 83 | | | | |
| 21 | 84 | 85 | 86 | 87 | |
| 23 | 88 | 89 | | | |
| 24 | 90 | | | | |
| 25 | 91 | | | | |
| 27 | 92 | 93 | | | |
| 29 | 94 | | | | |
| 31 | 95 | 96 | 97 | 98 | |
| 33 | 99 | 100 | | | |
| 35 | 101 | | | | |
| 36 | 102 | | | | |
| 38 | 103 | | | | |
| 39 | 104 | | | | |
| 40 | 105 | | | | |
| 41 | 106 | | | | |
| 42 | 107 | | | | |
| 43 | 108 | 109 | 110 | | |
| 44 | 111 | 112 | 113 | | |
| 46 | 114 | | | | |
| 47 | 115 | 116 | | | |
| 48 | 117 | | | | |
| 49 | 118 | 119 | | | |
| 50 | 120 | | | | |
| 51 | 121 | | | | |
| 52 | 123 | | | | |
| 53 | 126 | | | | |
| 54 | 128 | | | | |
| 55 | 129 | 130 | 131 | 132 | 133 |
| 56 | 134 | 135 | | | |
| 57 | 136 | | | | |
| 58 | 137 | | | | |
| 59 | 138 | 139 | | | |
| 61 | 140 | | | | |
| 62 | 141 | | | | |
| 63 | 142 | 143 | 144 | | |
| 64 | 145 | | | | |
| 66 | 146 | | | | |
| 67 | 147 | | | | |
| 149 | 162 | 163 | 164 | | |
| 150 | 165 | 166 | 167 | | |
| 151 | 168 | | | | |
| 152 | 172 | | | | |
| 153 | 174 | | | | |
| 154 | 177 | 178 | 179 | | |
| 155 | 180 | | | | |
| 156 | 183 | 184 | 185 | | |
| 157 | 187 | | | | |
| 158 | 190 | | | | |
| 159 | 192 | 193 | 194 | | |
| 160 | 195 | 196 | 197 | | |
| 161 | 198 | | | | |

The inventive nucleic acid sequences Seq. ID No. 2-67, 149-161, and 201-202 of the determined candidate genes and the determined amino acid sequences Seq. ID No. 71-148, and 162-200 are described in the following sequence protocol.

1. A nucleic acid sequence that codes a gene product or a part thereof, comprising
a) a nucleic acid sequence, selected from the group Seq. Id no. 3, 7, 9, 15, 17, 18, 21, 25, 27, 33, 35, 36, 38, 39, 40, 42, 43, 44, 46, 47, 48, 50, 51, 52, 53, 54, 55, 57, 58, 59, 61, 62, 63, 65, 66, 67, 150-153, 156-158, and 160-161
b) an allelic variation of the nucleic acid sequences named under a) or
c) a nucleic acid sequence that is complementary to the nucleic acid sequences named under a) or b).

2. A nucleic acid sequence according to one of the sequences Seq. ID No. 3, 7, 9, 15, 17, 18, 21, 25, 27, 33, 35, 36, 38, 39, 40, 42, 43, 44, 46, 47, 48, 50, 51, 52, 53, 54, 55, 57, 58, 59, 61, 62, 63, 65, 66, 67, 150-153, 156-158, 160-161 or a complementary or allelic variant thereof.

3. Nucleic acid sequence Seq. ID No. 2-67, 149-161, 201-202, characterized in that it is expressed elevated in breast tumor tissue.

4. BAC, PAC and Cosmid clones containing functional genes and their chromosomal localization according to sequences Seq. ID No. 2-67, 149-161, and 201-202 for use as a vehicle for gene transfer.

5. A nucleic acid sequence according to claims 1 to 4, wherein it has 90% homology to a human nucleic acid sequence.

6. A nucleic acid sequence according to claims 1 to 4, wherein it has 95% homology to a human nucleic acid sequence.

7. A nucleic acid sequence comprising a portion of the nucleic acid sequences named in claims 1 to 6, in such a sufficient amount that they hybridize with the sequences according to claims 1 to 6.

8. A nucleic acid sequence according to claims 1 to 7, wherein the size of the fragment has a length of at least 50 to 4500 bp.

9. A nucleic acid sequence according to claims 1 to 7, wherein the size of the fragment has a length of at least 150 to 4000 bp.

10. A nucleic acid sequence according to one of claims 1 to 9, which codes at least one partial sequence of a bioactive polypeptide.

11. An expression cassette, comprising a nucleic acid fragment or a sequence according to one of claims 1 to 9, together with at least one control or regulatory sequence.

12. An expression cassette, comprising a nucleic acid fragment or a sequence according to claim 11, in which the control or regulatory sequence is a suitable promoter.

13. An expression cassette according to one of claims 11 and 12, wherein the DNA sequences located on the cassette code a fusion protein, which comprises a known protein and a bioactive polypeptide fragment.

14. Use of nucleic acid sequences according to claims 1 to 10 for producing full-length genes.

15. A DNA fragment, comprising a gene, that can be obtained from the use according to claim 14.

16. Host cell, containing as the heterologous part of its expressible genetic information a nucleic acid fragment according to one of claims 1 to 10.

17. Host cell according to claim 16, wherein it is a prokaryotic or eukaryotic cell system.

18. Host cell according to one of claims 16 or 17, wherein the prokaryotic cell system is E. coli and the eukaryotic cell system is an animal, human or yeast cell system.

19. A process for producing a polypeptide or a fragment, wherein the host cells according to claims 16 to 18 are cultivated.

20. An antibody that is directed against a polypeptide or a fragment that is coded by the nucleic acids of sequences Seq. ID No. 2-67, 149-161, and 201-202, which can be obtained according to claim 19.

21. An antibody according to claim 20, wherein it is monoclonal.

22. Polypeptide partial sequences according to sequences Seq. ID No. 71-148, and 162-198.

23. Polypeptide partial sequences according to claim 22, with at least 80% homology to these sequences.

24. Polypeptide partial sequences according to claim 22, with at least 90% homology to these sequences.

25. Use of polypeptide partial sequences according to sequences Seq. ID No. 71-148, and 162-198 as tools for finding active ingredients against breast cancer.

26. Use of nucleic acid sequences according to sequences Seq. ID No. 2-67, 149-161 and 201-202 for expression of polypeptides that can be used as tools for finding active ingredients against breast cancer.

27. Use of nucleic acid sequences Seq. ID No. 2-67, 149-161, and 201-202 in sense or antisense form.

28. Use of polypeptide partial sequences Seq. ID No. 71-148 and 162-198 as pharmaceutical agents in gene therapy for treatment of breast cancer.

29. Use of polypeptide partial sequences Seq. ID No. 71-148, and 162-198 for the production of a pharmaceutical agent for treatment of breast cancer.

30. Pharmaceutical agent, containing at least one polypeptide partial sequence Seq. ID No. 71-148, and 162-198.

31. A nucleic acid sequence according to claims 1 to 10, wherein it is a genomic sequence.

32. A nucleic acid sequence according to claims 1 to 10, wherein it is an mRNA sequence.

33. Genomic genes, their promoters, enhancers, silencers, exon structure, intron structure and their splice variants, that can be obtained from cDNAs of sequences Seq. ID No. 2-67, 149-161 and 201-202.

34. Use of the genomic genes according to claim 33, together with suitable regulatory elements.

35. Use according to claim 34, wherein the regulatory element is a suitable promoter and/or enhancer.

36. A nucleic acid sequence according to claims 1 to 7, wherein the size of the fragment has a length of at least 450 to 3500 bp.
